SEQUENCE LISTING (43 sequences)

SEQ ID NO: 1 - DNA, 333 bp, Mus sp.
caaattgttatgtcccagtctccagcaatcctgtctgcatctccaggggagaaggtcacaatgacttgcagggccaggtcaagtgtgagttacatacactggtaccaacagaagccaggatcctcccccaaaccctggatttatgccacatccaacctggcttctggagtccctggtcgcttcagtggcagtgggtctgggacctcttactctctcacaatcaccagagtggaggctgaagatgctgccacttattactgccagcagtggagtagtaagccacccacgttcggaggggggaccaagctggaaatcaaacgtacggatgctgca

SEQ ID NO: 2 - DNA, 363 bp, Mus sp.
caggtccaactgcagcagcctggggctgagttggtgaggcctgggacttcagtgaagttgtcctgcaaggcttctggctacaccttcaccagctactggatgcactggataaaacagaggcctggacaaggccttgagtggatcggagtgattgatccttctgataattatactaagtacaatcaaaagtttaagggcaaggccacattgactgtagacacatcctccagcacagcctacatgcagctcagcagcctgacatctgaggactctgcggtctatttctgtgcaagagagggctactacggtagtagtccctggtttgcttactggggccaagggactctggtcactgtctcctca

SEQ ID NO: 3 - protein, 111 aa, Mus sp.
QIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRTDAA

SEQ ID NO: 4 - protein, 121 aa, Mus sp.
QVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSS

SEQ ID NO: 5 - DNA, 30 bp, Mus sp.
agggccaggtcaagtgtgagttacatacac

SEQ ID NO: 6 - DNA, 21 bp, Mus sp.
gccacatccaacctggcttct

SEQ ID NO: 7 - DNA, 27 bp, Mus sp.
cagcagtggagtagtaagccacccacg

SEQ ID NO: 8 - DNA, 15 bp, Mus sp.
agctactggatgcac

SEQ ID NO: 9 - DNA, 51 bp, Mus sp.
gtgattgatccttctgataattatactaagtacaatcaaaagtttaagggc

SEQ ID NO: 10 - DNA, 36 bp, Mus sp.
gagggctactacggtagtagtccctggtttgcttac

SEQ ID NO: 11 - protein, 11 aa, Mus sp.
RARSSVVSYIH

SEQ ID NO: 12 - protein, 7 aa, Mus sp.
ATSNLAS

SEQ ID NO: 13 - protein, 9 aa, Mus sp.
QQWSSKPPT

SEQ ID NO: 14 - protein, 5 aa, Mus sp.
SYWMH

SEQ ID NO: 15 - protein, 17 aa, Mus sp.
VIDPSDNYTKYNQKFKG

SEQ ID NO: 16 - protein, 12 aa, Mus sp.
EGYYGSSPWFAY

SEQ ID NO: 17 - protein, 291 aa, Mus sp.
MSGPFPAEPTKGPLAMQPAPKVNLKRTSSLVGPTQSFFMRESKALGAVQIMNGLFHITLGGLLMIPTGVFAPICLSVWYPLWGGIMYIISGSLLAAAAEKTSRKSLVKAKVIMSSLSLFAAISGIILSIMDILNMTLSHFLKMRRLELIQTSKPYVDIYDCEPSNSSEKNSPSTQYCNSIQSVFLGILSAMLISAFFQKLVTAGIVENEWKRMCTRSKSNVVLLSAGEKNEQTIKMKEEIIELSGVSSQPKNEEEIEIIPVQEEEEEEAEINFPAPPQEQESLPVENEIAP

SEQ ID NO: 18 - DNA, 23 bp, Artificial Sequence
  Chemically synthesized - Mouse CD20 primer
atgagtggacctttcccagcaga

SEQ ID NO: 19 - DNA, 32 bp, Artificial Sequence
  Chemically synthesized - Mouse CD20 primer
ttaaggagcgatctcattttccactggcaagg

SEQ ID NO: 20 - DNA, 36 bp, Artificial Sequence
  Chemically synthesized - mD20-CIEf  peptide tag primer
acagatctcaccatgagtggacctttcccagcagag

SEQ ID NO: 21 - DNA, 31 bp, Artificial Sequence
  Chemically synthesized - mD20-CIEr peptide tag primer
gtgctagcaggacgatctcattttccactgg

SEQ ID NO: 22 - DNA, 28 bp, Artificial Sequence
  Chemically synthesized - Mouse CD20 RT205 primer
caccatgagtggacctttcccagcagag

SEQ ID NO: 23 - DNA, 25 bp, Artificial Sequence
  Chemically synthesized - Mouse CD20 RT203 primer
aggagcgatctcattttccactggc

SEQ ID NO: 24 - protein, 20 aa, Mus sp.
CSHFLKMRRLELIQTSKPYV

SEQ ID NO: 25 - DNA, 31 bp, Artificial Sequence
  Chemically synthesized - JH-G1 immunglobulin degenerate primer
  misc_feature (20)..(20): n can be either a or c
  misc_feature (21)..(21): n can be either a or g
  misc_feature (27)..(27): n can be either a or g
gggggtgtcgtgctagctgnngagacngtga

SEQ ID NO: 26 - DNA, 21 bp, Artificial Sequence
  Chemically synthesized - VK5-3 immunoglobulin degenerate primer
  misc_feature (9)..(9): n can be either g ot t
  misc_feature (15)..(15): n can be either c or a
caaattgtnatgtcncagtct

SEQ ID NO: 27 - DNA, 21 bp, Artificial Sequence
  Chemically synthesized - VK5-1 VK region primer
caaattgttatgtcccagtct

SEQ ID NO: 28 - DNA, 21 bp, Artificial Sequence
  Chemically synthesized - VK5-1 VK region primer
caaattgttatgtcccagtct

SEQ ID NO: 29 - DNA, 29 bp, Artificial Sequence
  Chemically synthesized - VH5 V-gamma1 region primer
caggtccaactgcagcagcctggggctga

SEQ ID NO: 30 - protein, 21 aa, Artificial Sequence
  Chemically synthesized - 18B12 Antibody VH N-terminal sequence
QVQLQQPGAELVAPGTSVKLS

SEQ ID NO: 31 - protein, 21 aa, Artificial Sequence
  Chemcially synthesized - 18B12 Antibody VL N-terminal sequence
  MISC_FEATURE (11)..(11): Xaa can be any amino acid
  MISC_FEATURE (16)..(16): Xaa can be any amino acid
QIVMSQSKAIXSASPXEKVTM

SEQ ID NO: 32 - DNA, 324 bp, Mus sp.
caaattgttatgtcccagtctccagcaatcctgtctgcatctccaggggagaaggtcacaatgacttgcagggccaggtcaagtgtgagttacatacactggtaccaacagaagccaggatcctcccccaaaccctggatttatgccacatccaacctggcttctggagtccctggtcgcttcagtggcagtgggtctgggacctcttactctctcacaatcaccagagtggaggctgaagatgctgccacttattactgccagcagtggagtagtaagccacccacgttcggaggggggaccaagctggaaatcaaacgtgct

SEQ ID NO: 33 - protein, 108 aa, Mus sp.
QIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRA

SEQ ID NO: 34 - DNA, 1356 bp, Mus sp.
caggtccaactgcagcagcctggggctgagttggtgaggcctgggacttcagtgaagttgtcctgcaaggcttctggctacaccttcaccagctactggatgcactggataaaacagaggcctggacaaggccttgagtggatcggagtgattgatccttctgataattatactaagtacaatcaaaagtttaagggcaaggccacattgactgtagacacatcctccagcacagcctacatgcagctcagcagcctgacatctgaggactctgcggtctatttctgtgcaagagagggctactacggtagtagtccctggtttgcttactggggccaagggactctggtcactgtctcctcagccaaaacaacagccccatcggtatacccactggcccctgtgtgtggagatacaactggctcctcggtgactctaggatgcctggtcaagggttatttccctgagccagtgaccttgacctggaactctgggtcgctgtccagtggtgtgcacaccttcccagctgtcctgcagtctgacctctacaccctcagcagctcagtgactgtaaccagcagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 35 - protein, 451 aa, Mus sp.
QVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 36 - DNA, 642 bp, Mus sp.
caaattgttatgtcccagtctccagcaatcctgtctgcatctccaggggagaaggtcacaatgacttgcagggccaggtcaagtgtgagttacatacactggtaccaacagaagccaggatcctcccccaaaccctggatttatgccacatccaacctggcttctggagtccctggtcgcttcagtggcagtgggtctgggacctcttactctctcacaatcaccagagtggaggctgaagatgctgccacttattactgccagcagtggagtagtaagccacccacgttcggaggggggaccaagctggaaatcaaacgtgctgatgctgcaccaactgtatcgattttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 37 - protein, 213 aa, Mus sp.
QIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 38 - DNA, 993 bp, Mus sp.
gccaaaacaacagccccatcggtatacccactggcccctgtgtgtggagatacaactggctcctcggtgactctaggatgcctggtcaagggttatttccctgagccagtgaccttgacctggaactctgggtcgctgtccagtggtgtgcacaccttcccagctgtcctgcagtctgacctctacaccctcagcagctcagtgactgtaaccagcagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 39 - protein, 330 aa, Mus sp.
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 40 - DNA, 318 bp, Mus sp.
gatgctgcaccaactgtatcgattttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttga

SEQ ID NO: 41 - protein, 105 aa, Mus sp.
DAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 42 - DNA, 60 bp, Mus sp.
atgagggtccccgctcagctcctggggctcctgctgctctggctcccaggtgcacgatgt

SEQ ID NO: 43 - DNA, 57 bp, Mus sp.
atgggttggagcctcatcttgctcttccttgtcgctgttgctacgcgtgtcctgtcc

(19) United States
(12) Patent Application Publication
(10) Pub. No.: US 2007/0136826 A1
(43) Pub. Date: Jun. 14, 2007

(54) Anti-mouse CD20 antibodies and uses thereof

(75) Inventors: Robert Joseph Dunn, San Diego, CA (US); Elisabeth Mertsching, San Diego, CA (US); Robert Peach, San Diego, CA (US); Marilyn R. Kehry, San Diego, CA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec Inc., Cambridge, MA (US)

(21) Appl. No.: 11/633,079

(22) Filed: Dec. 4, 2006

Related U.S. Application Data
(60) Provisional application No. 60/741,491, filed on Dec. 2, 2005.
(60) Provisional application No. 60/849,433, filed on Oct. 5, 2006.
(60) Provisional application No. 60/783,060, filed on Mar. 17, 2006.

(51) Int. Cl. A01K 67/027; C07K 16/28; C07H 21/04; C12P 21/06; C12N 5/06

(52) U.S. Cl. 800/3; 800/18; 435/69.1; 435/326; 435/320.1; 530/388.22; 536/23.53

(57) ABSTRACT

The present invention relates to antibodies, or antigen binding fragments, variants, or derivatives thereof. In particular embodiments, the present invention relates to recombinant monoclonal antibodies, specific for mouse CD20. In addition, the present invention relates to nucleic acid molecules encoding such antibodies, or antigen binding fragments, variants, or derivatives thereof, and vectors and host cells comprising such nucleic acid molecules. The invention further relates to methods for producing the monoclonal antibodies or antigen binding fragments, variants, or derivatives thereof of the invention, and to methods of using these antibodies or antigen binding fragments, variants, or derivatives thereof, alone or in combination, in animal models of disease.

### CROSS-REFERENCE TO RELATED APPLICATIONS

This application claims the benefit of U.S. Provisional Application No. 60/741,491, filed Dec. 2, 2005, U.S. Provisional Application No. 60/783,060, filed Mar. 17, 2006, and U.S. Provisional Application No. 60/849,433, filed Oct. 5, 2006, the entire contents of each which are herein incorporated by reference.

### BACKGROUND OF THE INVENTION

1. Field of the Invention

The present invention is directed to antibodies against the mouse CD20 protein and methods of using the antibody in mouse models of disease, particularly B-cell disorders. The present invention is also directed to the use of IgG2a isotype antibodies (e.g., mouse IgG2a antibodies against mouse CD20) in animal models of disease.

2. Background of the Invention

The anti-CD20 antibody, rituximab, has demonstrated a remarkable success in treating B cell neoplasias in combination with other therapies, and has recently demonstrated efficacy in treatment of rheumatoid arthritis. This success has fostered an interest in expanding the clinical applications for anti-CD20, combining anti-CD20 therapy with other potentially synergistic drugs, and in further characterizing the in vivo effects and mechanism of B cell depletion. However, the difficulty and expense of performing combination studies with rituximab or exploring new disease indications in patients or non-human primates are high. Many potential therapies are tested in rodent disease models, being an attractive approach for both the length of time needed for a study as well as the cost savings over clinical experimentation in human disease.

In the adult mouse, most B cells are generated in the bone marrow from pluripotent hematopoietic stem cells. Their differentiation goes through several stages from pro-B to mature B cells. The very early B-lineage-restricted precursors, the pro-B cells (B220⁺ CD43⁺ IgM⁻), differentiate into pre-B cells (B220⁺ CD43⁻ IgM⁻), which then become immature B cells (B220⁺ CD43⁻ IgM⁺ IgD⁻), the first B cells to express a mature B cell receptor. The T1 B cell subset (B220⁺ IgM^{hi} IgD^{lo} CD21⁻ CD23⁻) differentiates from immature B cells that migrated to the spleen. T1 B cells then progress to T2 B cells (B220⁺ IgM^{hi} IgD^{hi} CD21⁺ CD23⁺), which become mature B cells (B220⁺ IgM^{lo} IgD^{hi} CD21⁺ CD23⁺). Some mature B cells form the Marginal Zone B cell subset in the spleen (B220⁺ IgM^{hi} IgD^{lo} CD21⁺ CD23⁻). The Bla (B220^{+/-} CD11b⁺ CD5⁺ IgM⁺) and B1b (B220^{+/-} CD11b⁺ CD5⁻ IgM⁺) subsets of B cells are found in the peritoneal cavity and are generated independently of the bone marrow.

Until recently antibodies specific for mouse CD20 have not been produced. One approach of engineering mice that express human CD20 has made a great degree of progress, but still has limitations in that the human CD20 transgenic mice must be backcrossed to each different mouse strain utilized in a disease model. Mouse CD20 could have low immunogenicity for generating antibodies. Uchida J, Lee Y, Hasegawa M, Liang Y, Bradney A, Oliver J A, Bowen K, Steeber D A, Haas K M, Poe J C, and Tedder T F. 2004. Mouse CD20 expression and function. Int. Immunol. 16:119-129. Alternatively it may be challenging to generate stable hybridoma cell lines that produce antibodies that bind to their own cell surfaces (mouse fusion partners express CD20). However, the advantages of working in mouse models and the opportunity to explore effects of B cell depletion in disease models, as well as in characterizing the resulting immune response defects, make the anti-mouse CD20 antibody approach useful.

Thus, there exists a need for a mouse monoclonal antibody to mouse CD20 that can deplete B cells in mice and can be used for testing in a variety of disease models, both alone and in combination with other therapeutic approaches. Furthermore, there exists a need for mouse antibodies of the IgG2a isotype that can act as functional equivalents to the human IgG1 antibodies isotype in animal models of disease.

### BRIEF SUMMARY OF THE INVENTION

In one embodiment, the present invention is directed to an isolated polynucleotide comprising SEQ ID NO:5, SEQ ID NO:6, and SEQ ID NO:7. The invention is further directed to an isolated polynucleotide comprising SEQ ID NO:8, SEQ ID NO:9, and SEQ ID NO:10 The invention is further directed to an isolated polynucleotide comprising a sequence having at least 90% identity to SEQ ID NO:1. The invention is further directed to an isolated polynucleotide comprising a sequence having at least 95% identity to SEQ ID NO:1. The invention is further directed to an isolated polynucleotide comprising SEQ ID NO.:1. The invention is further directed to an isolated polynucleotide comprising a sequence having at least 90% identity to SEQ ID NO:2; an isolated polynucleotide comprising a sequence having at least 95% identity to SEQ ID NO:2. The invention is further directed to an isolated polynucleotide comprising SEQ ID NO.:2. In a further embodiment, the present invention is directed to an isolated polynucleotide according to any of the above, which encodes a fusion polypeptide.

In one aspect, the present invention is directed to isolated polynucleotides as above, further comprising a sequence encoding an antibody light or heavy chain constant region, preferably from a mouse. In certain embodiments, the isolated polynucleotides and polypeptides of the present invention further comprise an Fc region. In particular embodiments, the Fc region is a mouse IgG region, and more particularly, an IgG region selected from the group consisting of IgG1, IgG2a, IgG2b, IgG2c, and IgG3. In a more particular embodiment, the IgG region is IgG2a.

In further embodiments, the present invention is directed to an isolated polynucleotide encoding a polypeptide having the sequence of SEQ ID No.: 3. In another embodiment, the present invention is directed to an isolated polynucleotide encoding a polypeptide having the sequence of SEQ ID No.:4.

In some embodiments, the present invention is also directed to an isolated polynucleotide comprising a sequence encoding a polypeptide having the V_{H} or V_{L} region of the 18B12 antibody, or variants thereof, and a sequence encoding a polypeptide having the sequence of an antibody constant region.

In certain embodiments, the present invention is directed to an isolated polynucleotide comprising a nucleic acid encoding an immunoglobulin heavy chain variable region (VH), wherein the CDR1, CDR2, and CDR3 regions of said VH are at least 90% identical, 95% identical or identical, respectively, to reference heavy chain CDR1, CDR2, and CDR3 sequences of SEQ ID NO:8, SEQ ID NO:9, and SEQ ID NO:10, wherein an antibody or antigen-binding fragment thereof comprising said VH specifically binds to mouse CD20. In other embodiments, the present invention is directed to an isolated polynucleotide comprising a nucleic acid encoding an immunoglobulin heavy chain variable region (VH), wherein the CDR1, CDR2, and CDR3 regions of said VH are at least 95% identical, respectively, to reference heavy chain CDR1, CDR2, and CDR3 sequences of SEQ ID NO:8, SEQ ID NO:9, and SEQ ID NO:10, wherein an antibody or antigen-binding fragment thereof comprising said VH specifically binds to mouse CD20. In a further embodiment, the present invention is directed to an isolated polynucleotide, wherein said CDR1, CDR2, and CDR3 regions of said VH, are encoded respectively, by a nucleic acid sequence of SEQ ID NO:8, SEQ ID NO:9, and SEQ ID NO:10. In a further embodiment, the present invention is directed to an isolated polynucleotide comprising a nucleic acid encoding a VH at least 90% identical or identical to, a reference VH sequence of SEQ ID NO:2 wherein an antibody or antigen-binding fragment thereof comprising said VH specifically binds to mouse CD20.

In other embodiments, the isolated polynucleotides of the present invention further comprise a nucleic acid encoding a signal peptide fused to said VH or VL. In further embodiments, the present invention is directed to isolated polynucleotide according to the above, wherein an antibody or antigen-binding fragment thereof comprising said VH or VL specifically binds to the same epitope as bound by the 18B12 antibody or competitively inhibits the 18B12 antibody from binding to mouse CD20. In particular embodiments the present invention is directed to isolated polynucleotides as above, wherein an antibody or antigen-binding fragment thereof comprising said VH or VL, or both, specifically binds to a mouse CD20 polypeptide or fragment thereof, or a mouse CD20 variant polypeptide, with an affinity characterized by a dissociation constant (K_{D}) no greater than 5×10⁻² M, 10² M, 5×10⁻³ M, 10³ M, 5×10⁻⁴ M, 10⁻⁴ M, 5×10⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In certain embodiments, the present invention is directed to an isolated polynucleotide comprising a nucleic acid encoding an immunoglobulin light chain variable region (VL), wherein the CDR1, CDR2, and CDR3 regions of said VL are at least 90% identical, 95% identical, or identical, respectively, to reference heavy chain CDR1, CDR2, and CDR3 sequences of SEQ ID NO:5, SEQ ID NO:6, and SEQ ID NO:7, wherein an antibody or antigen-binding fragment thereof comprising said VL specifically binds to mouse CD20. In another embodiment, the present invention is directed to an isolated polynucleotide, wherein said CDR1, CDR2, and CDR3 regions of said VL, are encoded respectively, by a nucleic acid sequence of SEQ ID NO:5, SEQ ID NO:6, and SEQ ID NO:7.

In a further embodiment, the present invention is directed to an isolated polynucleotide comprising a nucleic acid encoding a VL at least 90% identical or identical to the reference VL sequence of SEQ ID NO:1, wherein an antibody or antigen-binding fragment thereof comprising said VL specifically binds to mouse CD20. An isolated polynucleotide according to claim 38, wherein said VL is encoded by the nucleic acid sequence of SEQ ID NO:1.

In some embodiments, the isolated polynucleotide according to the present invention further comprise a heterologous polynucleotide.

The present invention is also directed to compositions comprising the polynucleotides of the present invention which encode a VH, and a VL, wherein an antibody or antigen-binding fragment thereof comprising said VH and said VL specifically binds to mouse CD20. In certain embodiments of the present invention, an antibody or antigen-binding fragment thereof comprising said VH, said VL, or both said VH and VL specifically binds to the major extracellular loop region of mouse CD20. In certain embodiments of the present invention, an antibody or antigen-binding fragment thereof comprising said VH, said VL, or both said VH and VL is a multivalent antibody molecule comprising at least two heavy chains and at least two light chains, or is multispecific, bispecific, monovalent, bivalent, polyvalent, or bifunctional.

In certain embodiments, the present invention is directed to an isolated polynucleotide comprising at least two CDRs of the 18B12 antibody. In a particular embodiment, said CDRs comprise at least two sequences selected from the group consisting of: SEQ ID NO:5, SEQ ID NO:6, SEQ ID NO:7, SEQ ID NO:8, SEQ ID NO:9, and SEQ ID NO:10. In another particular embodiment, the isolated polynucleotide encodes an antibody or antigen binding fragment thereof. In a further embodiment, the isolated polynucleotide of the present invention comprises at least three, four, five or six CDRs of the 18B12 antibody. In a particular embodiment, said CDRs comprises at least three sequences selected from the group consisting of: SEQ ID NO:5, SEQ ID NO:6, SEQ ID NO:7, SEQ ID NO:8, SEQ ID NO:9, and SEQ ID NO:10.

The present invention is also directed to vectors and host cells comprising the isolated polynucleotides of the present invention, and methods of producing an antibody or an antigen binding fragment thereof that is capable of specifically binding to mouse CD20, said method comprising culturing the host cell of the present invention in a medium under conditions allowing the expression of said polynucleotide encoding said antigen binding molecule; and recovering said antigen binding molecule from the resultant culture.

The present invention is also directed to an isolated polypeptide comprising the sequence of SEQ ID NO:3 or a variant thereof. An isolated polypeptide comprising the sequence of SEQ ID NO:4 or a variant thereof. In a further embodiment, the present invention is directed to an isolated polypeptide comprising a polypeptide having a sequence selected from the group consisting of: SEQ ID NO:11, SEQ ID NO:12 and SEQ ID NO:13; and a polypeptide having a sequence selected from the group consisting of: SEQ ID NO: 14, SEQ ID NO:15 and SEQ ID NO: 16. In additional embodiments, the present invention is directed to isolated polypeptides further comprising an Fc region, and more particularly, an IgG region. In certain embodiments, the IgG region is an IgG isotype selected from the group consisting of IgG1, IgG2a, IgG2b and IgG2c. In a particular embodiment, the IgG region is an IgG2a isotype.

The present invention is also directed to an isolated polypeptide comprising a sequence that is 90% identical to a reference sequence selected from the group consisting of SEQ ID NO: 13, SEQ ID NO:14, SEQ ID NO:15, and SEQ ID NO: 16. In another embodiment, the present invention is directed to an isolated polypeptide comprising a sequence that is 95% identical to a reference sequence selected from the group consisting of SEQ ID NO:11, SEQ ID NO: 13, SEQ ID NO:14, SEQ ID NO:15, and SEQ ID NO: 16. In a further embodiment, the present invention is directed to an isolated polypeptide comprising a sequence that is identical to a reference sequence selected from the group consisting of SEQ ID NO:11, SEQ ID NO: 13, SEQ ID NO:14, SEQ ID NO:15, and SEQ ID NO: 16.

In another embodiment, the present invention is directed to an isolated polynucleotide comprising a sequence that is 90% identical to a reference sequence selected from the group consisting of SEQ ID NO: 7, SEQ ID NO:8, SEQ ID NO:9, and SEQ ID NO: 10. In a further embodiment, the present invention is directed to an isolated polynucleotide comprising a sequence that is 95% identical to a reference sequence selected from the group consisting of SEQ ID NO:5, SEQ ID NO: 7, SEQ ID NO:8, SEQ ID NO:9, and SEQ ID NO: 10. In another embodiment, the present invention is directed to an isolated polynucleotide comprising a sequence that is identical to a reference sequence selected from the group consisting of SEQ ID NO:5, SEQ ID NO: 7, SEQ ID NO:8, SEQ ID NO:9, and SEQ ID NO: 10.

The present invention is further directed to an isolated polypeptide comprising SEQ ID NO:11, SEQ ID NO:12 and SEQ ID NO: 13. The present invention is also directed to an isolated polypeptide comprising SEQ ID NO:14, SEQ ID NO:15, and SEQ ID NO: 16. The present invention is further directed to an isolated polypeptide comprising SEQ ID NO:3 or SEQ ID NO:4 or a variant thereof. In further embodiments, the isolated polypeptide is a fusion polypeptide.

An isolated polypeptide comprising an immunoglobulin heavy chain variable region (VH), wherein the CDR1, CDR2, and CDR3 regions of said VH are at least 90% identical, 95% identical, or identical, respectively, to reference heavy chain CDR1, CDR2, and CDR3 sequences of SEQ ID NO:14, SEQ ID NO:15, and SEQ ID NO:16, wherein an antibody or antigen-binding fragment thereof comprising said VH specifically binds to mouse CD20. In another aspect, the present invention is directed to an isolated polypeptide according to claim 91 wherein said CDR1, CDR2, and CDR3 regions of said VH, are encoded respectively, by an amino acid sequence of SEQ ID NO:14, SEQ ID NO:15, and SEQ ID NO:16.

In another aspect, the present invention is directed to an isolated polypeptide comprising a nucleic acid encoding a VH at least 90% identical or identical to a reference VH sequence of SEQ ID NO:4 wherein an antibody or antigen-binding fragment thereof comprising said VH specifically binds to mouse CD20. In another embodiment, said VH is encoded by the amino acid sequence of SEQ ID NO:4. In a further embodiment, the isolated polypeptide further comprises a signal peptide fused to said VH. In a further embodiment, an antibody or antigen-binding fragment thereof comprising said VH specifically binds to the same epitope as bound by the 18B12 antibody. In another embodiment, an antibody or antigen-binding fragment thereof comprising said VH competitively inhibits the 18B12 antibody from binding to mouse CD20.

In another embodiment, the present invention further comprises an isolated polypeptide according to the present invention, wherein an antibody or antigen-binding fragment thereof comprising said VH specifically binds to a mouse CD20 polypeptide or fragment thereof, or a mouse CD20 variant polypeptide, with an affinity characterized by a dissociation constant (K_{D}) no greater than 5×10⁻² M, 10⁼² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In another aspect, the present invention is directed to an isolated polypeptide comprising an immunoglobulin light chain variable region (VL), wherein the CDR1, CDR2, and CDR3 regions of said VL are at least 90% identical, 95% identical, or identical, respectively, to reference heavy chain CDR1, CDR2, and CDR3 sequences of SEQ ID NO:11, SEQ ID NO:12, and SEQ ID NO:13, wherein an antibody or antigen-binding fragment thereof comprising said VL specifically binds to mouse CD20. In another embodiment, the CDR1, CDR2, and CDR3 regions of said VL, are encoded respectively, by an amino acid sequence of SEQ ID NO:11, SEQ ID NO:12, and SEQ ID NO:13.

In another embodiment, the present invention is further directed to an isolated polypeptide comprising a nucleic acid encoding a VL at least 90% identical or identical to the reference VL sequence of SEQ ID NO:1, wherein an antibody or antigen-binding fragment thereof comprising said VL specifically binds to mouse CD20. In another embodiment, said VL is encoded by the nucleic acid sequence of SEQ ID NO:1. In a further embodiment, the isolated polypeptide according to the present invention further comprises a nucleic acid encoding a signal peptide fused to said VL. In a further embodiment, an antibody or antigen-binding fragment thereof comprising said VL specifically binds to the same epitope as bound by the 18B12 antibody. In a further embodiment, an antibody or antigen-binding fragment thereof comprising said VL competitively inhibits the 18B12 antibody from binding to mouse CD20. In another embodiment , an antibody or antigen-binding fragment thereof comprising said VL specifically binds to a mouse CD20 polypeptide or fragment thereof, or a mouse CD20 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10¹⁴ M, 10⁻¹⁴ M, 5×10⁻¹⁵ M, or 10⁻¹⁵ M.

In another embodiment, the isolated polypeptide according to the present invention further comprises a heterologous polypeptide. In another aspect of the invention, said isolated polypeptide is conjugated to an agent selected from the group consisting of a therapeutic agent, a prodrug, a peptide, a protein, an enzyme, a virus, a lipid, a biological response modifier, a phannaceutical agent, or PEG.

The present invention is also directed to antibodies or antigen binding fragments thereof comprising the isolated polypeptides of the present invention or encoded by the polynucleotides of the present invention. In one embodiment, the antibody is a whole antibody. In another embodiment, said antigen binding fragment is an antibody fragment. In certain embodiments, the antibody or antigen-binding fragment thereof is capable of competing with the 18B12 antibody for specific binding to mouse CD20. In certain embodiments the antibody or antigen binding fragment thereof comprises an Fc region. In a particular embodiment, the Fc region is an IgG Fc region, and more particularly, said IgG Fc region is selected from the group consisting of IgG1, IgG2b and IgG2c. In a more particular embodiment, the IgG Fc region is IgG2a.

In one aspect, the present invention is directed to an antibody or antigen binding fragment thereof comprising at least two CDRs of the 18B12 antibody. In another embodiment, the antibody or antigen binding fragment thereof according to the present invention comprises at least three CDRs of the 18B12 antibody. In a further embodiment, said antigen binding molecule comprises the variable region of an antibody light or heavy chain.

The present invention is further directed to a pharmaceutical test composition comprising the polynucleotides and polypeptides of the present invention.

The present invention is further directed to a method of depleting B cells in a non-human subject, said method comprising administering to said non-human subject an amount of a composition comprising the isolated polypeptide, compositions, antibodies or antigen binding fragments thereof, or the pharmaceutical test compositions of the present invention. In one embodiment, the methods of the present invention further comprise observing the effects of B cell depletion on the non-human subject. In one embodiment, the non-human subject is an animal model of disease. In a particular embodiment, the animal model is a mouse model. In certain embodiments, observing the effects of B cell depletion comprises a measurement selected from the group consisting of: measuring the number of B-cells, measuring tumor size, and measuring serum concentration of a protein or molecule. In certain embodiments, the animal model of disease is a model for a human disease selected from the group consisting of solid tumors such as sarcomas, carcinomas (e.g., colon carcinoma, renal cell carcinoma, adenocarcinoma), and lymphomas (e.g., B cell lymphoma, T cell lymphoma), thymoma, epithelial carcinogenesis, collagen-induced arthritis, serum transfer arthritis, rheumatoid arthritis, mast cell-mediated inflammation, multiple sclerosis, systemic lupus erythrematosus, liver fibrosis, lung fibrosis, and kidney fibrosis.

The invention is further directed to methods of determining the effects of B-cell depletion in an animal model of disease and methods of testing therapeutic agents for use in treating diseases or disorders, the methods comprising: administering to said animal model of disease an amount of a composition comprising the isolated polypeptides, the compositions, the antibodies or antigen binding fragments thereof, or the pharmaceutical test compositions of the present invention; and observing the effects of administration on said animal model of disease.

In certain embodiments, observing the effects of B cell depletion comprises a measurement selected from the group consisting of: measuring the number of B-cells, measuring tumor size, measuring serum concentration of a protein or molecule, and measuring urine concentration of a protein or molecule. In particular embodiments, the animal model of disease is a model for a human disease selected from the group consisting of solid tumors such as sarcomas, carcinomas (e.g., colon carcinoma, renal cell carcinoma, adenocarcinoma), and lymphomas (e.g., B cell lymphoma, T cell lymphoma), thymoma, epithelial carcinogenesis, collagen-induced arthritis, serum transfer arthritis, rheumatoid arthritis, mast cell-mediated inflammation, multiple sclerosis, systemic lupus erythrematosus, liver fibrosis, lung fibrosis, and kidney fibrosis. In particular embodiments of the methods of the present invention, the polypeptide, antibody or antigen binding fragment thereof, composition, or pharmaceutical test composition is administered in an amount of about 1 mg/kg to about 20 mg/kg. In more particular embodiments, the polypeptide, antibody or antigen binding fragment thereof, composition, or pharmaceutical test composition is administered in an amount selected from the group consisting of about 1 mg/kg/, about 2mg/kg, about 5 mg/kg, about 10 mg/kg, and about 20 mg/kg.

In further embodiments, the polypeptide, antibody or antigen binding fragment thereof, composition, or pharmaceutical test composition is administered intravenously, intraperitoneally, or subcutaneously. In a preferred embodiment, the polypeptide, antibody or antigen binding fragment thereof, composition, or pharmaceutical test composition is administered intravenously. In further embodiments, the polypeptide, antibody or antigen binding fragment thereof, composition, or pharmaceutical test composition is administered once daily, once weekly, twice weekly, every other week or once per month. In a preferred embodiment, the polypeptide, antibody or antigen binding fragment thereof, composition, or pharmaceutical test composition is administered every other week. In certain embodiments of the methods of the present invention, said polypeptide, antibody or antigen binding fragment thereof, composition, or pharmaceutical test composition is administered with a second composition. In certain embodiments, said second composition comprises an agent selected from the group consisting of an anti-CD19 antibody, an anti-CD21 antibody, an anti-CD22 antibody, an anti-CD23 antibody, an anti-CD80 antibody, a chemotherapeutic agent, Toll receptor antagonists, BR3-Fc, anti-BAFF, anti-adhesion molecule antibodies, an anti-ICAM antibody (e.g., anti-ICAM-1, anti-ICAM-2, or anti-ICAM-3), an anti-LFA-1 antibody, an anti-CD11 a antibody, an anti-α4 integrin antibody, lymphotoxin beta receptor antagonists, LTβR-Ig, anti-CD40 ligand (CD154), anti-inflammatory agents, and a combination thereof.

The 18B12-A1C3-H3 clone was deposited with the American Type Culture Collection (“ATCC”) on Dec. 22, 2005, and was given the ATCC Deposit Number PTA-7299. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure. Thus, in one aspect, the present invention is directed to a hybridoma cell line ATCC No. PTA-7299. In another aspect, the present invention is directed to a monoclonal antibody produced by ATCC No. PTA-7299. In one embodiment, the present invention is directed to a monoclonal antibody that specifically binds to mouse CD20 and is produced by ATCC No. PTA-7299. In another embodiment, the present invention is directed to a monoclonal antibody that binds to the same epitope of mouse CD20 as the monoclonal antibody produced by ATCC No. PTA-7299. In a further aspect, the present invention is directed to a pharmaceutical test composition comprising the antibody produced by ATCC No. PTA-7299, or an antigen binding fragment thereof. In another aspect, the present invention is directed to a method of depleting B cells in a non-human subject, the method comprising administering to a non-human subject an amount of a composition comprising the monoclonal antibody produced by ATCC No. PTA-7299, or an antigen binding fragment thereof.

### BRIEF DESCRIPTION OF THE DRAWINGS/FIGURES

FIG. 1 The 18B12 Antibody Recognizes Mouse CD20. The indicated mouse B cell lines or splenocytes were stained with 18B12 IgG2b switch variant antibody and a secondary PE-conjugated monoclonal antibody specific for mouse IgG2b as described in the Examples herein. Gray filled histograms, unstained; dashed-line histograms, secondary PE reagent alone; dotted-line histograms, isotype control; solid-line histograms, 18B12. Secondary staining reagents were tested for low background staining of B cell surface immunoglobulin.

FIG. 2. Competition Binding of 18B12 Isotype Switch Variants on Mouse CD20 Transfected 300.18 Cells. Mouse CD20 transfected 300.18 cells were stained with 18B12 isotypes at 1 μg/ml (IgG1, closed diamond (♦), closed square (▪); IgG2b, closed triangle (▴), open square (□); IgG2c, open triangle, (Δ), open diamond (⋄)) and detected with biotin-conjugated isotype-specific monoclonal antibodies in the presence or absence of various concentrations of competitor antibodies as described in the Examples herein. Staining with APC-streptavidin was quantified in a FACSArray Bioanalyzer.

FIG. 3. Five Week B Cell Depletion after Two Doses of 18B12 IgGI. Male C57B1/6 mice (4 per group) were treated with either 18B12 IgGI or 2B8 (mouse IgG1 isotype control) (10 mg/kg i.v. on days 0 and 14) or left untreated. Five weeks after the first antibody dose animals were retroorbitally bled for peripheral blood and then sacrificed for harvest of lymph nodes, spleen, bone marrow, and peritoneal wash. B cell subsets were stained and analyzed as described in the Examples herein.

FIG. 4. B Cell Depletion by Different 18B12 Isotypes. Male C57B1/6 mice (4 per group) were treated with either 18B12 IgG1, IgG2c, IgG2b or isotype-matched control antibodies (10 mg/kg i.v. on day 0) or left untreated. One, three, seven, and 14 days after the antibody dose animals were retroorbitally bled for peripheral blood and then sacrificed for harvest of spleen. B cell subsets were stained and analyzed as described in the Examples herein.

FIG. 5. Time Course of B Cell Repopulation after 18B12 IgG1 Treatment. Male C57B1/6 mice (4 per group) were treated with 18B12 IgG1 or isotype-matched control antibodies (10 mg/kg i.v. on day 0, or both on days 0 and 14) or left untreated. Starting 3 weeks after the first dose and every two weeks thereafter mice were retroorbitally bled for peripheral blood analyses (A) and then sacrificed for harvest of spleen (B) and bone marrow (C) (shown in B and C only for mice dosed on days 0 and 14). B cell subsets were stained and analyzed as described in the Examples herein. Bars represent the percentage of CD19⁺cells relative to the total CD3⁺ plus CD19⁺ cells (A) or the percentage of B cells in each subset relative to that present in untreated mice (B, C). The large error bars in the week 9 results in (C) derive from one outlier mouse out of the 4 total mice in the treatment group.

FIG. 6. Day 7 B Cell Depletion after Combination Treatment with 18B12 IgG1 and BR3-Fc. Male C57B1/6 mice (4 per group) were treated either with PBS, 18B12 IgGI plus C2B8 (human IgG1 isotype control), BR3:Fc plus 2B8 (mouse IgG1 isotype control), or 18B12 plus BR3:Fc. The 18B12 and 2B8 antibodies were dosed at 10 mg/kg i.v. on day 0 and the BR3:Fc fusion protein and C2B8 antibody were dosed at 8 mg/kg i.v. on day 0. Seven days after treatment animals were sacrificed for harvest of spleen (A), bone marrow (B), and peritoneal wash (not shown). B cell subsets were stained and analyzed as described in the Examples herein.

FIG. 7. Pharmacokinetic Analysis of Single Dose 18B12 Administration. Male C57B1/6 mice (4 per group) were dosed either with 18B12 IgG1, IgG2b, or IgG2c at 10 mg/kg i.v. Various times after treatment animals were sacrificed for harvest of serum. Quantification of 18B12 isotype level was on the mouse CD20 transfected 300.18 B cell line relative to a standard curve of the isotype being quantified as described in the Examples herein. 18B12 IgG1, (•); 18B12 IgG2b, (∘); 18B 12 IgG2c, (▪).

FIG. 8. Staining of Splenic CD 19⁺ B Cells with Different Isotypes of 18B12 Anti-Mouse CD20. Spleen cells harvested from either a wild type C57BL/6 or a CD20 knockout (k/o) mouse were stained with PE-anti-CD19 and with unlabeled IgG1, IgG2b, or IgG2a anti-CD20 (18B12) antibodies or the corresponding isotype controls. Bound antibodies were detected with biotinylated anti-mouse isotype-specific monoclonal antibodies and APC-streptavidin. Dead cells were stained with the dye, 7-AAD, prior to analysis by flow cytometry. The histograms shown were derived from gated viable CD19⁺ B cells.

FIG. 9. Splenic B Cell Depletion in BALB/c Mice Treated with Different 18B12 Isotypes. Male BALB/c mice (3 per group) were given a single dose (10 mg/kg i.v.) of either an isotype variant of 18B12 (IgG1, IgG2b, or IgG2a) or a corresponding isotype control antibody. On days 1, 3, 7, and 14 post dosing mice were sacrificed, spleens harvested, and cells analyzed by flow cytometry for mature (A), marginal zone (B), T2 (C), or T1 (D) B cell subsets. Staining and reagents were as described in the Examples herein. Each bar is the mean ± standard deviation of absolute cell counts from 3 mice.

FIG. 10. Peritoneal Cavity B Cell Depletion in BALB/c Mice Treated with Different 18B12 Isotypes. Male BALB/c mice (3 per group) were given a single dose (10 mg/kg i.v.) of either an isotype variant of 18B12 (IgG1, IgG2b, or IgG2a) or a corresponding isotype control. On days 3, 7, 14, and 22 post dosing mice were sacrificed, a peritoneal lavage was performed, and cells were analyzed by flow cytometry for B2 (A), B1a (B), or B1b (C) B cell subsets. Staining and reagents were as described in the Examples herein. Each bar is the mean ± standard deviation of absolute cell counts from 3 mice.

### DETAILED DESCRIPTION OF THE INVENTION

Definitions

It is to be noted that the term “a” or “an” entity refers to one or more of that entity; for example, “an anti-mouse CD20 antibody” or “an 18B12 antibody” is understood to represent one or more anti-mouse CD20 or 18B12 antibodies. Likewise, “an IgG2a antibody” is understood to represent one or more IgG2a antibodies.” As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

Also included as polypeptides of the present invention are fragments, derivatives, analogs, or variants of the foregoing polypeptides, and any combination thereof. The terms “fragment,” “variant,” “derivative” and “analog” when referring to anti-mouse CD20 antibodies or antibody polypeptides or IgG2a antibodies or antibody polypeptides of the present invention include any polypeptides which retain at least some of the antigen-binding properties of the corresponding native antibody or polypeptide. Fragments of polypeptides of the present invention include proteolytic fragments, as well as deletion fragments, in addition to specific antibody fragments discussed elsewhere herein. Variants of anti-mouse CD20 antibodies and antibody polypeptides of the present invention include fragments as described above, and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions or additions. Derivatives of anti-mouse CD20 antibodies and antibody polypeptides or mouse IgG2a antibodies and antibody polypeptides (e.g., aniti-mouse CD20 IgG2a antibodies) of the present invention, are polypeptides which have been altered so as to exhibit additional features not found on the native polypeptide. Examples include fusion proteins. Variant polypeptides may also be referred to herein as “polypeptide analogs.” As used herein a “derivative” of an anti-mouse CD20 antibody or antibody polypeptide or IgG2a antibody or antibody polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an anti-mouse CD20 antibody (or, e.g., a mouse IgG2a antibody) contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding an anti-mouse CD20 antibody or fragment, variant, or derivative thereof (or an IgG2a antibody or fragment, variant, or derivative thereof). Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is RNA, for example, in the form of messenger RNA (mRNA).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length”polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

The present invention is directed to certain anti-mouse CD20 antibodies (also referred to herein as “mouse CD20 antibodies”), or antigen-binding fragments, variants, or derivatives thereof. Unless specifically referring to full-sized antibodies such as naturally-occurring antibodies, the term “anti-mouse CD20 antibodies” or “mouse CD20 antibodies” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

The present invention is also directed to certain mouse IgG2a antibodies, or antigen binding fragments, variants, or derivatives thereof, and uses of the mouse IgG2a antibodies. By “mouse IgG2a antibody” is meant an antibody, or antigen binding fragment, variant, or derivative thereof, directed to a mouse target antigen and comprising a heavy chain constant region, or fragment or variant or derivative thereof, of the IgG2a isotype.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are well understood.

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1−γ4). It is the nature of this chain that determines the “class” of the antibody as IgG, IgM, IgA IgG, or IgE, respectively. The immunoglobulin subclasses (isotypes) e.g., IgG₁, IgG₂ₐ, IgG_{2b}, IgG₂, IgG₃, etc. are well characterized and are known to confer functional specialization. Modified versions of each of these classes and isotypes are readily discernable to the skilled artisan in view of the instant disclosure and, accordingly, are within the scope of the instant invention. All immunoglobulin classes are clearly within the scope of the present invention, the following discussion will generally be directed to the IgG class of immunoglobulin molecules. With regard to IgG, a standard immunoglobulin molecule comprises two identical light chain polypeptides, and two identical heavy chain polypeptides. The four chains are typically joined by disulfide bonds in a “Y” configuration wherein the light chains bracket the heavy chains starting at the mouth of the “Y” and continuing through the variable region.

In mice, the various IgG subclasses show a hierarchy of activities in vivo, with IgG2a and IgG2b being the greatest in protective and pathogenic activities. Nimmerjahn et al. 2005. Immunity 23:41-51. The mouse IgG2a heavy chain is thought to be a functional equivalent of the human IgG1 heavy chain, and has strong effector functions in vivo. In vitro, mouse IgG2a binds to FcγRIV with moderate affinity, which is approximately 100-fold higher than the affinity with which mouse IgG1 binds to mouse FcγRIII. Nimmerjahn et al. 2005. Immunity 23:41-51.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (C_{L}) and the heavy chain (C_{H}1, C_{H}2 or CH_{H}3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the C_{H}3 and C_{L} domains actually comprise the carboxy-terminus of the heavy and light chain, respectively.

As indicated above, the variable region allows the antibody to selectively recognize and specifically bind epitopes on antigens. That is, the V_{L} domain and V_{H} domain of an antibody combine to form the variable region that defines a three dimensional antigen binding site. This quaternary antibody structure forms the antigen binding site present at the end of each arm of the Y. More specifically, the antigen binding site is defined by three complementary determining regions (CDRs) on each of the V_{H} and V_{L} chains. In some instances, e.g., certain immunoglobulin molecules derived from camelid species or engineered based on camelid immunoglobulins, a complete immunoglobulin molecule may consist of heavy chains only, with no light chains.

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,”Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J Mol. Biol. 196:901-917 (1987), which are incorporated herein by reference in their entireties, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR DEFINITIONS¹

| | Kabat | Chothia |
|---|---|---|
| V_{H} CDR1 | 31-35 | 26-32 |
| V_{H} CDR2 | 50-65 | 52-58 |
| V_{H} CDR3 | 95-102 | 95-102 |
| V_{L} CDR1 | 24-34 | 26-32 |
| V_{L} CDR2 | 50-56 | 50-52 |
| V_{L} CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an anti-mouse CD20 antibody or antigen-binding fragment, variant, or derivative thereof of the present invention are according to the Kabat numbering system.

Antibodies or antigen-binding fragments, variants, or derivatives thereof of the invention include, but are not limited to, monoclonal antibodies, multivalent antibodies, multispecific antibodies, single chain antibodies, epitope-binding fragments, e.g., Fab fragments, Fab′ fragments and F(ab′)₂ fragments, Fd fragments, Fv fragments, single-chain Fv fragments (scFv), single-chain antibodies, and disulfide-linked Fv fragments (sdFv). ScFv molecules, for example, are known in the art and are described, e.g., in U.S. Pat. No. 5,892,019. Methods of making multispecific (e.g., bispecific) antibodies comprising synthetic connecting peptides are described, e.g., in US 2005/0163782A1, PCT Publ. Nos. WO 2006/074399 A2 and WO 2005/000899 A2, and U.S. Nos. 60/783,622 and 60/812,688, each of which is incorporated by reference herein in its entirety. Immunoglobulin or antibody molecules of the invention can be of any type (e.g., IgG, IgE, IgM, IgD, IgA, and IgY), class or subclass (e.g., IgG1, IgG2a, IgG2b, IgG2c, and IgG3) of immunoglobulin molecule. In a particular embodiment, immunoglobulin or antibody molecules of the invention are IgG2a antibodies.

Antibody fragments, including single-chain antibodies, may comprise the variable region(s) alone or in combination with the entirety or a portion of the following: hinge region, C_{H}1, C_{H}2, and C_{H}3 domains. Also included in the invention are antigen-binding fragments also comprising any combination of variable region(s) with a hinge region, C_{H}1, C_{H}2, and C_{H}3 domains.

As used herein, the term “heavy chain portion” includes amino acid sequences derived from an immunoglobulin heavy chain. A polypeptide comprising a heavy chain portion comprises at least one of: a C_{H}1 domain, a hinge (e.g., upper, middle, and/or lower hinge region) domain, a C_{H}2 domain, a C_{H}3 domain, or a variant or fragment thereof. For example, a binding polypeptide for use in the invention may comprise a polypeptide chain comprising a C_{H}1 domain; a polypeptide chain comprising a C_{H}1 domain, at least a portion of a hinge domain, and a C_{H}2 domain; a polypeptide chain comprising a C_{H}1 domain and a CH³ domain; a polypeptide chain comprising a C_{H}1 domain, at least a portion of a hinge domain, and a C_{H}3 domain, or a polypeptide chain comprising a C_{H}1 domain, at least a portion of a hinge domain, a C_{H}2 domain, and a C_{H}3 domain. In another embodiment, a polypeptide of the invention comprises a polypeptide chain comprising a C_{H}3 domain. Further, a binding polypeptide for use in the invention may lack at least a portion of a C_{H}2 domain (e.g., all or part of a C_{H}2 domain). As set forth above, it will be understood by one of ordinary skill in the art that these domains (e.g., the heavy chain portions) may be modified such that they vary in amino acid sequence from the naturally occurring immunoglobulin molecule.

In certain anti-mouse CD20 antibodies, or antigen-binding fragments, variants, or derivatives thereof disclosed herein, the heavy chain portions of one polypeptide chain of a multimer are identical to those on a second polypeptide chain of the multimer. Alternatively, heavy chain portion-containing monomers of the invention are not identical. For example, each monomer may comprise a different target binding site, forming, for example, a bispecific antibody.

The heavy chain portions of a binding polypeptide for use in the diagnostic and treatment methods disclosed herein may be derived from different immunoglobulin molecules. For example, a heavy chain portion of a polypeptide may comprise a C_{H}1 domain derived from an IgG1 molecule and a hinge region derived from an IgG3 molecule. In another example, a heavy chain portion can comprise a hinge region derived, in part, from an IgG1 molecule and, in part, from an IgG3 molecule.

As used herein, the term “light chain portion” includes amino acid sequences derived from an immunoglobulin light chain. Preferably, the light chain portion comprises at least one of a V_{L} or C_{L} domain.

Anti-mouse CD20 antibodies, or antigen-binding fragments, variants, or derivatives thereof disclosed herein may be described or specified in terms of the epitope(s) or portion(s) of an antigen, e.g., a target polypeptide (mouse CD20) that they recognize or specifically bind. The portion of a target polypeptide which specifically interacts with the antigen binding domain of an antibody is an “epitope,” or an “antigenic determinant.” A target polypeptide may comprise a single epitope, but typically comprises at least two epitopes, and can include any number of epitopes, depending on the size, conformation, and type of antigen. Furthermore, it should be noted that an “epitope” on a target polypeptide may be or include non-polypeptide elements, e.g., an “epitope may include a carbohydrate side chain.

The minimum size of a peptide or polypeptide epitope for an antibody is thought to be about four to five amino acids. Peptide or polypeptide epitopes preferably contain at least seven, more preferably at least nine and most preferably between at least about 15 to about 30 amino acids. Since a CDR can recognize an antigenic peptide or polypeptide in its tertiary form, the amino acids comprising an epitope need not be contiguous, and in some cases, may not even be on the same peptide chain. In the present invention, peptide or polypeptide epitope recognized by anti-mouse CD20 antibodies of the present invention contains a sequence of at least 4, at least 5, at least 6, at least 7, more preferably at least 8, at least 9, at least 10, at least 15, at least 20, at least 25, or between about 15 to about 30 contiguous or non-contiguous amino acids of mouse CD20.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

By way of non-limiting example, an antibody may be considered to bind a first epitope preferentially if it binds said first epitope with a dissociation constant (K_{D}) that is less than the antibody's K_{D} for the second epitope. In another non-limiting example, an antibody may be considered to bind a first antigen preferentially if it binds the first epitope with an affinity that is at least one order of magnitude less than the antibody's K_{D} for the second epitope. In another non-limiting example, an antibody may be considered to bind a first epitope preferentially if it binds the first epitope with an affinity that is at least two orders of magnitude less than the anitibody's K_{D} for the second epitope.

In another non-limiting example, an antibody may be considered to bind a first epitope preferentially if it binds the first epitope with an off rate (k(off)) that is less than the antibody's k(off) for the second epitope. In another non-limiting example, an antibody may be considered to bind a first epitope preferentially if it binds the first epitope with an affinity that is at least one order of magnitude less than the antibody's k(off) for the second epitope. In another non-limiting example, an antibody may be considered to bind a first epitope preferentially if it binds the first epitope with an affinity that is at least two orders of magnitude less than the antibody's k(off) for the second epitope.

An antibody or antigen-binding fragment, variant, or derivative disclosed herein may be said to bind a target polypeptide disclosed herein or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. More preferably, an antibody of the invention may be said to bind a target polypeptide disclosed herein or a fragment or variant thereof with an off rate (k(off)) less than or equal to 5×10⁻⁴ sec⁻¹, ₁₀⁻⁴ sec^{-1,} 5×10⁻⁵ sec⁻¹, or 10⁻⁵ sect⁻¹ 5×10⁻⁶ sec⁻¹, 10⁻⁶ sec⁻¹, 5×10⁻⁷ sec⁻¹ or 10⁻⁷ sect⁻¹.

An antibody or antigen-binding fragment, variant, or derivative disclosed herein may be said to bind a target polypeptide disclosed herein or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, an antibody of the invention may be said to bind a target polypeptide disclosed herein or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×106 M⁻¹ sec⁻¹ or 10⁷ M⁻¹ sec⁻¹.

An antibody is said to competitively bind or competitively inhibit binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

An antibody is said to competitively bind or competitively inhibit binding of a reference antibody to a target polypeptide if it preferentially binds to that target polypeptide to the extent that it blocks, to some degree, binding of the reference antibody to the target polypeptide. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

In one example, a method of determining competitive binding comprises the following method (See, e.g., Harlow et al., ANTIBODIES: A LABORATORY MANUAL, Cold Spring Harbor Laboratory Press (2nd ed. 1988) at pages 567-569):
1. Purify and label each of the monoclonal antibodies to be studied.
2. Bind the standard antigen solution to the bottom of the wells of a PVC microtitre plate by adding 50 microliters of antigen solution to each well (20 μg/ml). PVC binds about 100 ng/well (300 ng/cm2). Use at least 1 μg/well if maximal binding is necessary. Dilutions can be done in PBS, if not otherwise required by experimental design. Avoid introducing extraneous proteins or compounds that could lower the binding capacity of the PVC. The amount of antigen should be titrated to the lowest level necessary for strong signal. The amount of competitor needed will increase as the amount of antigen bound to the solid phase increases.
3. Incubate at room temperature for 2 hours with humidity.
4. Wash the plate 2× with PBS.
5. Saturate the remaining protein binding sites on the PVC plate by incubating with blocking buffer (3% BSA/PBS with 0.02% sodium azide). Incubate at room temperature with humidity from 2 hours to overnight.
6. Wash the plate 2× with PBS.
7. Add a mixture of two antibodies to be tested, one labeled, one unlabeled. Incubate at room temperature for 2 hours with humidity. Perform antibody dilutions in 3% BSA/PBS with 0.02% sodium azide. Do not use buffers with azide when using a horse radish peroxidase detection system. For optimization, titrate the amount of labeled antibody and use at a subsaturating level. For accurate quantitations, titrate the amount of unlabeled competitor and compare the midpoints of the competition curves.
8. Wash 4× with PBS to remove unbound antibodies.

As used herein, the term “affinity” refers to a measure of the strength of the binding of an individual epitope with the CDR of an immunoglobulin molecule. As used herein, the term “avidity” refers to the overall stability of the complex between a population of immunoglobulins and an antigen, that is, the functional combining strength of an immunoglobulin mixture with the antigen. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

Anti-mouse CD20 antibodies or antigen-binding fragments, variants or derivatives thereof of the invention may also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, may actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain epitope, if it does not bind any other analog, ortholog, or homolog of that epitope.

Anti-mouse CD20 antibodies (or IgG2a antibodies) or antigen-binding fragments, variants or derivatives thereof of the invention may also be described or specified in terms of their binding affinity to a polypeptide of the invention. Preferred binding affinities include those with a dissociation constant or K_{D} less than 5×10⁻² M, 10⁻² M, 5×10⁻³ M, 10⁻³ M, 5×10⁻⁴ M, 10⁻⁴ M, 5×10⁻⁵M, 10⁻⁵ M, 5×10⁶ M, 10-6 M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Anti-mouse CD20 antibodies (or IgG2a antibodies) or antigen-binding fragments, variants or derivatives thereof of the invention may be “multispecific,” e.g., bispecific, trispecific or of greater multispecificity, meaning that it recognizes and binds to two or more different epitopes present on one or more different antigens (e.g., proteins) at the same time. Thus, whether an antibody is “monospecfic” or “multispecific,” e.g., “bispecific,” refers to the number of different epitopes with which a binding polypeptide reacts. Multispecific antibodies may be specific for different epitopes of a target polypeptide described herein or may be specific for a target polypeptide as well as for a heterologous epitope, such as a heterologous polypeptide or solid support material.

As used herein the term “valency” refers to the number of potential binding domains, e.g., antigen binding domains, present in an anti-mouse CD20 antibody or binding polypeptide, or IgG2a antibody or binding polypeptide. Each binding domain specifically binds one epitope. When an anti-mouse CD20 antibody, binding polypeptide or antibody comprises more than one binding domain, each binding domain may specifically bind the same epitope, for an antibody with two binding domains, termed “bivalent monospecific,” or to different epitopes, for an antibody with two binding domains, termed “bivalent bispecific.” An antibody may also be bispecific and bivalent for each specificity (termed “bispecific tetravalent antibodies”). In another embodiment, tetravalent minibodies or domain deleted antibodies can be made.

Bispecific bivalent antibodies, and methods of making them, are described, for instance in U.S. Pat. Nos. 5,731,168; 5,807,706; 5,821,333; and U.S. Appl. Publ. Nos. 2003/020734 and 2002/0155537, the disclosures of all of which are incorporated by reference herein. Bispecific tetravalent antibodies, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J Immunol. 148:1547-1553 (1992). Methods of making multispecific (e.g., bispecific) antibodies comprising synthetic connecting peptides are described, e.g., in US 2005/0163782A1, PCT Publ. Nos. WO 2006/074399 A2 and WO 2005/000899 A2, and U.S. Nos. 60/783,622 and 60/812,688. Each of these disclosures is incorporated by reference herein in its entirety.

As previously indicated, the subunit structures and three dimensional configuration of the constant regions of the various imrunoglobulin classes are well known. As used herein, the term “V_{H} domain” includes the amino terminal variable domain of an immunoglobulin heavy chain and the term “C_{H}1 domain” includes the first (most amino terminal) constant region domain of an immunoglobulin heavy chain. The C_{H}1 domain is adjacent to the V_{H} domain and is amino terminal to the hinge region of an immunoglobulin heavy chain molecule.

As used herein the term “C_{H}2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat EA et al. op. cit. The C_{H}2 domain is unique in that it is not closely paired with another domain. Rather, two N-linked branched carbohydrate chains are interposed between the two C_{H}2 domains of an intact native IgG molecule. It is also well documented that the C_{H}3 domain extends from the C_{H}2 domain to the C-terminal of the IgG molecule and comprises approximately 108 residues.

As used herein, the term “hinge region” includes the portion of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains.

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the C_{H}1 and C_{L} regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant invention) is obtained from a second species.

As used herein the term “properly folded polypeptide” includes polypeptides (e.g., anti-mouse CD20 antibodies) in which all of the functional domains comprising the polypeptide are distinctly active. As used herein, the term “improperly folded polypeptide” includes polypeptides in which at least one of the functional domains of the polypeptide is not active. In one embodiment, a properly folded polypeptide comprises polypeptide chains linked by at least one disulfide bond and, conversely, an improperly folded polypeptide comprises polypeptide chains not linked by at least one disulfide bond.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence. For example, polynucleotides encoding the CDRs of an immunoglobulin variable region may be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product, and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “treat” or “treatment” refer to both therapeutic treatment and prophylactic or preventative measures, wherein the object is to prevent or slow down (lessen) an undesired physiological change or disorder, such as the progression of B-cell lymphoma or multiple sclerosis. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. “Treatment” according to the present invention also includes taking measures to prevent or slow a physiological change or disorder in an animal model of disease.

As used herein, phrases such as “simulating treatment of human disease” refers to therapeutic, prophylactic, or preventative measures taken in a non-human animal model of disease to determine the effects of the treatment in the disease model. In one embodiment, the effects observed in the non-human animal model are used to predict the effects of treatment in a human. In one embodiment, the present invention is directed to a method of simulating treatment of a human disease with an IgG1 isotype human antibody to a human protein comprising administering to a non-human animal model of disease an IgG2a antibody that specifically binds to a target antigen that is homologous to the human antigen to which the human antibody binds. In a particular embodiment, the non-human animal model of disease is a mouse. In another particular embodiment, the IgG2a antibody is an anti-mouse CD20 antibody.

As used herein, such phrases as “animal model of disease” and “animal disease model” are meant to include both in vivo and in vitro systems (e.g., cell lines, as well as live non-human animals such as mice, e.g., transgenic mice) that mimic, display symptoms of, or otherwise represent an analogous or similar disease or disorder that occurs, e.g., in another species, or in a whole organ, tissue, or animal, (where the model is, is for example, a cell line). In one embodiment, the disease or disorder to which the animal model of disease corresponds is a human disease or disorder. In one embodiment, the animal model of disease comprises a mouse model of a human disease or disorder.

By “subject” or “individual” or “mammal,” is meant any subject, particularly a mammalian subject, for or of whom diagnosis, prognosis, therapy, test therapy, study, and/or research is desired. Mammalian subjects include humans, domestic animals, farm animals, and zoo, sports, or pet animals such as dogs, cats, guinea pigs, rabbits, rats, mice, horses, cattle, cows, and so on. In one embodiment, the subject is a non-human animal. In a more particular embodiment, the subject is a mouse model of disease.

As used herein, the phrase, “pharmaceutical test composition” refers to a composition comprising the antibodies, or antigen binding fragments, variants, or derivatives thereof administered to a model of animal disease.

As used herein, phrases such as “a subject that would benefit from administration of an anti-mouse CD20 antibody” and “an animal in need of treatment” includes subjects, such as mammalian subjects, including, e.g., mouse models of disease, that would benefit from or yield information about the effects of administration of an anti-mouse CD20 antibody used, e.g., for detection of a mouse CD20 polypeptide (e.g., for a diagnostic procedure) and/or from treatment, e.g., palliation or prevention of a disease such as B-cell lymphoma, EAE, SLE, or any other disease model in mice, with an anti-mouse CD20 antibody. As described in more detail herein, the anti-mouse CD20 antibody can be used in unconjugated form or can be conjugated, e.g., to a drug, prodrug, or an isotope.

Mouse CD20

In both human and mouse, CD20 is a membrane-embedded protein that passes through the membrane four times. The human and mouse CD20 proteins are predicted to be 73% identical, with the regions of greatest similarity occurring in the transmembrane region. There is less conservation of sequence in the extracellular region between the mouse and human CD20 proteins (Polyak and Deans, 2002. Blood 99: 3256-3262, herein incorporated by reference in its entirety).

Mouse CD20 is a protein of about 291 amino acids as set forth in SEQ ID NO: 17, below (see also, Accession No. P19437):

| | |
|---|---|
| 1 | MSGPFPAEPT KGPLAMQPAP KVNLKRTSSL VGPTQSFFMR |
| | ESKALGAVQI |
| 51 | MNGLFHITLG GLLMIPTGVF APICLSVWYP LWGGIMYIIS |
| | GSLLAAAAEK |
| 101 | TSRKSLVKAK VIMSSLSLFA AISGIILSIM DILNMTLSHF |
| | LKMRRLELIQ |
| 151 | TSKPYVDIYD CEPSNSSEKN SPSTQYCNSI QSVFLGILSA |
| | MLISAFFQKL |
| 201 | VTAGIVENEW KRMCTRSKSN VVLLSAGEKN EQTIKMKEEI |
| | IELSGVSSQP |
| 251 | KNEEEIEIIP VQEEEEEEAE INFPAPPQEQ ESLPVENEIA |
| | P |

The region of SEQ ID NO:17 as shown above in bold text represents the major extracellular loop of mouse CD20 (i.e., predicted to be from about residue 134 to about residue 182). The anti-human CD20 antibodies that have been developed (e.g., Rituximab, B1, 2H7, and 1F5) have been mapped to bind epitopes in the extracellular region (Polyak and Deans, 2002. Blood 99: 3256-3262). The underlined asparagine (“N”) residues represent potential glycosylation sites in the mouse CD20 protein.

Anti-Mouse CD20 Antibodies

In one embodiment, the present invention is directed to mouse anti-CD20 antibodies, or antigen-binding fragments, variants, or derivatives thereof. For example, the present invention includes at least the antigen-binding domains of the 18B12 monoclonal antibody, and fragments, variants, and derivatives thereof.

As used herein, the term “antigen binding domain” includes a site that specifically binds an epitope on an antigen (e.g., an epitope of mouse CD20). The antigen binding domain of an antibody typically includes at least a portion of an immunoglobulin heavy chain variable region and at least a portion of an immunoglobulin light chain variable region. The binding site formed by these variable regions determines the specificity of the antibody.

The present invention is more specifically directed to an anti-mouse CD20 antibody (also reffered to interchangeably herein as a “mouse CD20 antibody”), or antigen-binding fragment, variant or derivative thereof, where the anti-mouse CD20 antibody or fragment or variant thereof is the 18B12 antibody or binds to the same epitope as the 18B12 monoclonal antibody.

The invention is further drawn to an anti-mouse CD20 antibody, or antigen-binding fragment, variant or derivatives thereof, where the anti-mouse CD20 antibody or antigen-binding fragment, variant or derivative thereof competitively inhibits the 18B12 monoclonal antibody.

The invention is also drawn to an anti-mouse CD20 antibody, or antigen-binding fragment, variant or derivatives thereof, where the anti-mouse CD20 antibody comprises at least the antigen binding region of the 18B12 monoclonal antibody.

The nucleotide sequence of the VL region of the 18B12 antibody is represented by SEQ ID NO:1, and is shown below:

| | |
|---|---|
| 1 | CAAATTGTTA TGTCCCAGTC TCCAGCAATC CTGTCTGCAT |
| | CTCCAGGGGA |
| 51 | GAAGGTCACA ATGACTTGCA GGGCCAGGTC AAGTGTGAGT |
| | TACATACACT |
| 101 | GGTACCAACA GAAGCCAGGA TCCTCCCCCA AACCCTGGAT |
| | TTATGCCACA |
| 151 | TCCAACCTGG CTTCTGGAGT CCCTGGTCGC TTCAGTGGCA |
| | GTGGGTCTGG |
| 201 | GACCTCTTAC TCTCTCACAA TCACCAGAGT GGAGGCTGAA |
| | GATGCTGCCA |
| 251 | CTTATTACTG CCAGCAGTGG AGTAGTAAGC CACCCACGTT |
| | CGGAGGGGGG |
| 301 | ACCAAGCTGG AAATCAAACG TACGGATGCT GCA |

In another embodiment, the nucleotide sequence of the VL region of the anti-mouse CD20 antibody of the present invention is represented by SEQ ID NO:32, and is shown below:

| | |
|---|---|
| 1 | CAAATTGTTA TGTCCCAGTC TCCAGCAATC CTGTCTGCAT |
| | CTCCAGGGGA |
| 51 | GAAGGTCACA ATGACTTGCA GGGCCAGGTC AAGTGTGAGT |
| | TACATACACT |
| 101 | GGTACCAACA GAAGCCAGGA TCCTCCCCCA AACCCTGGAT |
| | TTATGCCACA |
| 151 | TCCAACCTGG CTTCTGGAGT CCCTGGTCGC TTCAGTGGCA |
| | GTGGGTCTGG |
| 201 | GACCTCTTAC TCTCTCACAA TCACCAGAGT GGAGGCTGAA |
| | GATGCTGCCA |
| 251 | CTTATTACTG CCAGCAGTGG AGTAGTAAGC CACCCACGTT |
| | CGGAGGGGGG |
| 301 | ACCAAGCTGG AAATCAAACG TGCT |

The amino acid sequence of the VL region of the 18B12 antibody is represented by SEQ ID NO:3, and is shown below:

| | |
|---|---|
| 1 | QIVMSQSPAI LSASPGEKVT MTCPARSSVS YIHWYQQKPG |
| | SSPKPWIYAT |
| 51 | SNLASGVPGR FSGSGSGTSY SLTITRVEAE DAATYYCQQW |
| | SSKPPTFGGG |
| 101 | TKLEIKRTDA A |

In another embodiment, the amino acid sequence of the VL region of the anti-mouse CD20 antibody of the present invention is represented by SEQ ID NO:33, and is shown below:

| | |
|---|---|
| 1 | QIVMSQSPAI LSASPGEKVT MTCRARSSVS YIHWYQQKPG |
| | SSPKPWIYAT |
| 51 | SNLASGVPGR FSGSGSGTSY SLTITRVEAE DAATYYCQQW |
| | SSKPPTFGGG |
| 101 | TKLEIKRA |

The nucleotide sequence of the VH region of the 18B12 antibody is represented by SEQ SEQ ID NO:2, and is shown below:

| | |
|---|---|
| 1 | CAGGTCCAAC TGCAGCAGCC TGGGGCTGAG TTGGTGAGGC |
| | CTGGGACTTC |
| 51 | AGTGAAGTTG TCCTGCAAGG CTTCTGGCTA CACCTTCACC |
| | AGCTACTGGA |
| 101 | TGCACTGGAT AAAACAGAGG CCTGGACAAG GCCTTGAGTG |
| | GATCGGAGTG |
| 151 | ATTGATCCTT CTGATAATTA TACTAAGTAC AATCAAAAGT |
| | TTAAGGGCAA |
| 201 | GGCCACATTG ACTGTAGACA CATCCTCCAG CACAGCCTAC |
| | ATGCAGCTCA |
| 251 | GCAGCCTGAC ATCTGAGGAC TCTGCGGTCT ATTTCTGTGC |
| | AAGAGAGGGC |
| 301 | TACTACGGTA GTAGTCCCTG GTTTGCTTAC TGGGGCCAAG |
| | GGACTCTGGT |
| 351 | CACTGTCTCC TCA |

The amino acid sequence of the VH region of the 18B12 antibody is represented by SEQ SEQ ID NO:4, and is shown below:

| | |
|---|---|
| 1 | QVQLQQPGAE LVRPGTSVKL SCKASGYTFT SYWMHWIKQR |
| | PGQGLEWIGV |
| 51 | IDPSDNYTKY NQKFKGKATL TVDTSSSTAY MQLSSLTSED |
| | SAVYFCAREG |
| 101 | YYGSSPWFAY WGQGTLVTVS S |

In certain embodiments, the anti-mouse CD20 antibodies, or antigen-binding fragment, variant, or derivative thereof of the present invention further comprise an Fc region. In one embodiment, the Fc region is an IgG region. The sequences of mouse constant regions, including IgG regions (e.g., IgG1, IgG2a, IgG2b, IgG2c, and IgG3) are known to those of skill in the art, and can be obtained and/or determined, e.g., from Kabat et al., U.S. Dept. of Health and Human Services, “Sequence of Proteins of Immunological Interest” (1983). In one embodiment, the IgG region or the Kappa region of the anti-mouse CD20 antibodies, or antigen-binding fragment, variant, or derivative thereof of the present invention are from a C57B1/6 (Igh-b alloytype) background (e.g., the 18B12 antibody). In another embodiment, the IgG2a constant region or the kappa constant region of the anti-mouse CD20 antibodies or antigen binding fragment, variant, or derivative thereof of the present invention are of the “a” allotype.

In one embodiment, the IgG2a constant region is encoded by a nucleotide sequence comprising the sequence of SEQ ID NO:38, which is shown below:

| | |
|---|---|
| 1 | GCCAAAACAA CAGCCCCATC GGTATACCCA CTGGCCCCTG |
| | TGTGTGGAGA |
| 51 | TACAACTGGC TCCTCGGTGA CTCTAGGATG CCTGGTCAAG |
| | GGTTATTTCC |
| 101 | CTGAGCCAGT GACCTTGACC TGGAACTCTG GGTCGCTGTC |
| | CAGTGGTGTG |
| 151 | CACACCTTCC CAGCTGTCCT GCAGTCTGAC CTCTACACCC |
| | TCAGCAGCTC |
| 201 | AGTGACTGTA ACCAGCAGCA CCTGGCCCAG CCAGTCCATC |
| | ACCTGCAATG |
| 251 | TGGCCCACCC GGCAAGCAGC ACCAAGGTGG ACAAGAAAAT |
| | TGAGCCCAGA |
| 301 | GGGCCCACAA TCAAGCCCTG TCCTCCATGC AAATGCCCAG |
| | CACCTAACCT |
| 351 | CTTGGGTGGA CCATCCGTCT TCATCTTCCC TCCAAAGATC |
| | AAGGATGTAC |
| 401 | TCATGATCTC CCTGAGCCCC ATAGTCACAT GTGTGGTGGT |
| | GGATGTGAGC |
| 451 | GAGGATGACC CAGATGTCCA GATCAGCTGG TTTGTGAACA |
| | ACGTGGAAGT |
| 501 | ACACACAGCT CAGACACAAA CCCATAGAGA GGATTACAAC |
| | AGTACTCTCC |
| 551 | GGGTGGTCAG TGCCCTCCCC ATCCAGCACC AGGACTGGAT |
| | GAGTGGCAAG |
| 601 | GAGTTCAAAT GCAAGGTCAA CAACAAAGAC CTCCCAGCGC |
| | CCATCGAGAG |
| 651 | AACCATCTCA AAACCCAAAG GGTCAGTAAG AGCTCCACAG |
| | GTATATGTCT |
| 701 | TGCCTCCACC AGAAGAAGAG ATGACTAAGA AACAGGTCAC |
| | TCTGACCTGC |
| 751 | ATGGTCACAG ACTTCATGCC TGAAGACATT TACGTGGAGT |
| | GGACCAACAA |
| 801 | CGGGAAAACA GAGCTAAACT ACAAGAACAC TGAACCAGTC |
| | CTGGACTCTG |
| 851 | ATGGTTCTTA CTTCATGTAC AGCAAGCTGA GAGTGGAAAA |
| | GAAGAACTGG |
| 901 | GTGGAAAGAA ATAGCTACTC CTGTTCAGTG GTCCACGAGG |
| | GTCTGCACAA |
| 951 | TCACCACACG ACTAAGAGCT TCTCCCGGAC TCCGGGTAAA |
| | TGA |

In one embodiment, the IgG2a constant region is encoded by a polypeptide sequence comprising the sequence of SEQ ID NO:39, which is shown below:

| | |
|---|---|
| 1 | AKTTAPSVYP LAPVCGDTTG SSVTLGCLVK GYFPEPVTLT |
| | WNSGSLSSGV |
| 51 | HTFPAVLQSD LYTLSSSVTV TSSTWPSQSI TCNVAHPASS |
| | TKVDKKIEPR |
| 101 | GPTIKPCPPC KCPAPNLLGG PSVFIFPPKI KDVLMISLSP |
| | IVTCVVVDVS |
| 151 | EDDPDVQISW FVNNVEVHTA QTQTHREDYN STLRVVSALP |
| | IQHQDWMSGK |
| 201 | EFKCKVNNKD LPAPIERTIS KPKGSVRAPQ VYVLPPPEEE |
| | MTKKQVTLTC |
| 251 | MVTDFMPEDI YVEWTNNGKT ELNYKNTEPV LDSDGSYFMY |
| | SKLRVEKKNW |
| 301 | VERNSYSCSV VHEGLHNHHT TKSFSRTPGK |

In one embodiment, the kappa constant region is encoded by a nucleotide sequence comprising the sequence of SEQ ID NO:40, which is shown below:

| | |
|---|---|
| 1 | GATGCTGCAC CAACTGTATC GATTTTCCCA CCATCCAGTG |
| | AGCAGTTAAC |
| 51 | ATCTGGAGGT GCCTCAGTCG TGTGCTTCTT GAACAACTTC |
| | TACCCCAAAG |
| 101 | ACATCAATGT CAAGTGGAAG ATTGATGGCA GTGAACGACA |
| | AAATGGCGTC |
| 151 | CTGAACAGTT GGACTGATCA GGACAGCAAA GACAGCACCT |
| | ACAGCATGAG |
| 201 | CAGCACCCTC ACGTTGACCA AGGACGAGTA TGAACGACAT |
| | AACAGCTATA |
| 251 | CCTGTGAGGC CACTCACAAG ACATCAACTT CACCCATTGT |
| | CAAGAGCTTC |
| 301 | AACAGGAATG AGTGTTGA |

In one embodiment, the kappa constant region is encoded by a polypeptide sequence comprising the sequence of SEQ ID NO:41, which is shown below:

| | |
|---|---|
| 1 | DAAPTVSIFP PSSEQLTSGG ASVVCFLNNF YPKDINVKWK |
| | IDGSERQNGV |
| 51 | LNSWTDQDSK DSTYSMSSTL TLTKDEYERH NSYTCEATHK |
| | TSTSPIVKSF |
| 101 | NRNEC |

In some embodiments, the heavy and light chain sequences of the present invention further comprise a leader sequence. In one embodiment, the leader sequence of the light chain is encoded by a nucleotide sequence comprising the sequence of SEQ ID NO:42, which is shown below:

| | |
|---|---|
| 1 | ATGAGGGTCC CCGCTCAGCT CCTGGGGCTC CTGCTGCTCT |
| | GGCTCCCAGG |
| 51 | TGCACGATGT |

In one embodiment, the leader sequence of the light chain is encoded by a nucleotide sequence comprising the sequence of SEQ ID NO:42, which is shown below:

| | |
|---|---|
| 1 | ATGGGTTGGA GCCTCATCTT GCTCTTCCTT GTCGCTGTTG |
| | CTACGCGTGT |
| 51 | CCTGTCC |

In one embodiment, the present invention comprises an immuoglobulin heavy chain encoded by a nucleotide sequence comprising the sequence of SEQ ID NO: 34, which is shown below:

CAGGTCCAACTGCAGCAGCCTGGGGCTGAGTTGGTGAGGCCTGGGACTTCAGTGAAGTTGTCCTG
CAAGGCTTCTGGCTACACCTTCACCAGCTACTGGATGCACTGGATAAAACAGAGGCCTGGACAAG
GCCTTGAGTGGATCGGAGTGATTGATCCTTCTGATAATTATACTAAGTACAATCAAAAGTTTAAG
GGCAAGGCCACATTGACTGTAGACACATCCTCCAGCACAGCCTACATGCAGCTCAGCAGCCTGAC
ATCTGAGGACTCTGCGGTCTATTTCTGTGCAAGAGAGGGCTACTACGGTAGTAGTCCCTGGTTTG
CTTACTGGGGCCAAGGGACTCTGGTCACTGTCTCCTCAGCCAAAACAACAGCCCCATCGGTATAC
CCACTGGCCCCTGTGTGTGGAGATACAACTGGCTCCTCGGTGACTCTAGGATGCCTGGTCAAGGG
TTATTTCCCTGAGCCAGTGACCTTGACCTGGAACTCTGGGTCGCTGTCCAGTGGTGTGCACACCT
TCCCAGCTGTCCTGCAGTCTGACCTCTACACCCTCAGCAGCTCAGTGACTGTAACCAGCAGCACC
TGGCCCAGCCAGTCCATCACCTGCAATGTGGCCCACCCGGCAAGCAGCACCAAGGTGGACAAGAA
AATTGAGCCCAGAGGGCCCACAATCAAGCCCTGTCCTCCATGCAAATGCCCAGCACCTAACCTCT
TGGGTGGACCATCCGTCTTCATCTTCCCTCCAAAGATCAAGGATGTACTCATGATCTCCCTGAGC
CCCATAGTCACATGTGTGGTGGTGGATGTGAGCGAGGATGACCCAGATGTCCAGATCAGCTGGTT
TGTGAACAACGTGGAAGTACACACAGCTCAGACACAAACCCATAGAGAGGATTACAACAGTACTC
TCCGGGTGGTCAGTGCCCTCCCCATCCAGCACCAGGACTGGATGAGTGGCAAGGAGTTCAAATGC
AAGGTCAACAACAAAGACCTCCCAGCGCCCATCGAGAGAACCATCTCAAAACCCAAAGGGTCAGT
AAGAGCTCCACAGGTATATGTCTTGCCTCCACCAGAAGAAGAGATGACTAAGAAACAGGTCACTC
TGACCTGCATGGTCACAGACTTCATGCCTGAAGACATTTACGTGGAGTGGACCAACAACGGGAAA
ACAGAGCTAAACTACAAGAACACTGAACCAGTCCTGGACTCTGATGGTTCTTACTTCATGTACAG
CAAGCTGAGAGTGGAAAAGAAGAACTGGGTGGAAAGAAATAGCTACTCCTGTTCAGTGGTCCACG
AGGGTCTGCACAATCACCACACGACTAAGAGCTTCTCCCGGACTCCGGGTAAATGA

In one embodiment, the present invention comprises an immunoglobulin heavy chain encoded by a polypeptide sequence comprising the sequence of SEQ ID NO: 35, which is shown below:

QVQLQQPGAELVRPGTSVKLSCKASGYTFTSYWMHWIKQRPGQGLEWIGVIDPSDNYTKYNQKFK
GKATLTVDTSSSTAYMQLSSLTSEDSAVYFCAREGYYGSSPWFAYWGQGTLVTVSSAKTTAPSVY
PLAPVCGDTTGSSVTLGCLVKGYFPEPVTLTWNSGSLSSGVHTFPAVLQSDLYTLSSSVTVTSST
WPSQSITCNVAHPASSTKVDKKIEPRGPTIKPCPPCKCPAPNLLGGPSVFIFPPKIKDVLMISLS
PIVTCVVVDVSEDDPDVQISWFVNNVEVHTAQTQTHREDYNSTLRVVSALPIQHQDWMSGKEFKC
KVNNKDLPAPIERTISKPKGSVPAPQVYVLPPPEEEMTKKQVTLTCMVTDFMPEDIYVEWTNNGK
TELNYKNTEPVLDSDGSYFMYSKLRVEKKNWVERNSYSCSVVHEGLHNHHTTKSFSRTPGK

In one embodiment, the present invention comprises an immunoglobulin light chain encoded by a nucleotide sequence comprising the sequence of SEQ ID NO: 36, which is shown below:

CAAATTGTTATGTCCCAGTCTCCAGCAATCCTGTCTGCATCTCCAGGGGAGAAGGTCACAATGAC
TTGCAGGGCCAGGTCAAGTGTGAGTTACATACACTGGTACCAACAGAAGCCAGGATCCTCCCCCA
AACCCTGGATTTATGCCACATCCAACCTGGCTTCTGGAGTCCCTGGTCGCTTCAGTGGCAGTGGG
TCTGGGACCTCTTACTCTCTCACAATCACCAGAGTGGAGGCTGAAGATGCTGCCACTTATTACTG
CCAGCAGTGGAGTAGTAAGCCACCCACGTTCGGAGGGGGGACCAAGCTGGAAATCAAACGTGCTG
ATGCTGCACCAACTGTATCGATTTTCCCACCATCCAGTGAGCAGTTAACATCTGGAGGTGCCTCA
GTCGTGTGCTTCTTGAACAACTTCTACCCCAAAGACATCAATGTCAAGTGGAAGATTGATGGCAG
TGAACGACAAAATGGCGTCCTGAACAGTTGGACTGATCAGGACAGCAAAGACAGCACCTACAGCA
TGAGCAGCACCCTCACGTTGACCAAGGACGAGTATGAACGACATAACAGCTATACCTGTGAGGCC
ACTCACAAGACATCAACTTCACCCATTGTCAAGAGCTTCAACAGGAATGAGTGTTGA

In one embodiment, the present invention comprises an immunoglobulin light chain encoded by a polypeptide sequence comprising the sequence of SEQ ID NO: 37, which is shown below:

QIVMSQSPAILSASPGEKVTMTCRARSSVSYIHWYQQKPGSSPKPWIYAT
SNLASGVPGRFSGSGSGTSYSLTITRVEAEDAATYYCQQWSSKPPTFGGG
TKLEIKRADAAPTVSIFPPSSEQLTSGGASVVCFLNNFYPKDINVKWKID
GSERQNGVLNSWTDQDSKDSTYSMSSTLTLTKDEYERHNSYTCEATHKTS
TSPIVKSFNRNEC

In certain aspects, the present invention is directed to a hybridoma cell line identified as American Type Culture Collection (ATCC) No. PTA-7299. In another aspect, the present invention is directed to a monoclonal antibody produced by the hybridoma cell line designated as ATCC No. PTA-7299. which was deposited with the ATCC on Dec. 22, 2005. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

In one embodiment, the present invention is directed to a monoclonal antibody that specifically binds to mouse CD20 and is produced by ATCC No. PTA-7299. In another embodiment, the present invention is directed to a monoclonal antibody that binds to the same epitope of mouse CD20 as the monoclonal antibody produced by ATCC No. PTA-7299.

In a further aspect, the present invention is directed to a pharmaceutical test composition comprising the antibody produced by ATCC No. PTA-7299, or an antigen binding fragment thereof. In another aspect, the present invention is directed to a method of depleting B cells in a non-human subject, the method comprising administering to a non-human subject an amount of a composition comprising the monoclonal antibody produced by ATCC No. PTA-7299, or an antigen binding fragment thereof.

In certain aspects, the present invention is directed to an antibody, or antigen-binding fragment, variant, or derivative thereof which specifically binds to a mouse CD20 polypeptide or fragment thereof, or a mouse CD20 variant polypeptide, with an affinity characterized by a dissociation constant (K_{D}) which is less than the K_{D} for said reference monoclonal antibody.

In certain embodiments, the present invention is directed to an antibody, or antigen-binding fragment, variant, or derivative thereof which specifically or preferentially binds to a particular mouse CD20 polypeptide fragment or domain. Such mouse CD20 polypeptide fragments include, but are not limited to, a mouse CD20 polypeptide comprising, consisting essentially of, or consisting of amino acids 133-142, 134-143, 135-144, 136-145, 137-146, 138-147, 139-148, 140-149, 141-150, 142-151, 143-152, 144-153, 145-154, 146-155, 147-156, 148-157, 149-158, 150-159, 151-160, 152-161, 153-162, 154-163, 155-164, 156-165, 157-166, 158-167, 159-168, 160-169, 161-170, 162-171, 163-172, 164-173, 165-174, 166-175, 167-176, 168-177, 169-178, 170-179, 171-180, 172-181, 173-182, 174-183, 175-184, 176-185, 177-186, 178-187, 179-188, 180-189, 181-190, 182-191, 183-192, 184-193, 185-194, and 186-195 of SEQ ID NO:17. Corresponding fragments of a variant mouse CD20 polypeptide at least 70%, 75%, 80%, 85%, 90%, or 95% identical to any of these amino acids regions are also contemplated.

In another embodiment, such mouse CD20 polypeptide fragments include, but are not limited to, a mouse CD20 polypeptide comprising, consisting essentially of, or consisting of amino acids 133-141, 134-142, 135-143, 136-144, 137-145, 138-146, 139-147, 140-148, 141-149, 142-150, 143-151, 144-152, 145-153, 146-154, 147-155, 148-156, 149-157, 150-158, 151-159, 152-160, 153-161, 154-162, 155-163, 156-164, 157-165, 158-166, 159-167, 160-168, 161-169, 162-170, 163-171, 164-172, 165-173, 166-174, 167-175, 168-176, 169-177, 170-178, 171-179, 172-180, 173-181, 174-182, 175-183, 176-184, 177-185, 178-186, 179-187, 180-188, 181-189, 182-190, 183-191, 184-192, 185-193, and 186-194 of SEQ ID NO:17. Corresponding fragments of a variant mouse CD20 polypeptide at least 70%, 75%, 80%, 85%, 90%, or 95% identical to any of these amino acids regions are also contemplated.

In another embodiment, such mouse CD20 polypeptide fragments include, but are not limited to, a mouse CD20 polypeptide comprising, consisting essentially of, or consisting of amino acids 133-140, 134-141, 135-142, 136-143, 137-144, 138-145, 139-146, 140-147, 141-148, 142-149, 143-150, 144-151, 145-152, 146-153, 147-154, 148-155, 149-156, 150-157, 151-158, 152-159, 153-160, 154-161, 155-162, 156-163, 157-164, 158-165, 159-166, 160-167, 161-168, 162-169, 163-170, 164-171, 165-172, 166-173, 167-174, 168-175, 169-176, 170-177, 171-178, 172-179, 173-180, 174-181, 175-182, 176-183, 177-184, 178-185, 179-186, 180-187, 181-188, 182-189, 183-190, 184-191, 185-192, and 186-193 of SEQ ID NO:17. Corresponding fragments of a variant mouse CD20 polypeptide at least 70%, 75%, 80%, 85%, 90%, or 95% identical to any of these amino acids regions are also contemplated.

In another embodiment, such mouse CD20 polypeptide fragments include, but are not limited to, a mouse CD20 polypeptide comprising, consisting essentially of, or consisting of amino acids 133-139, 134-140, 135-141, 136-142, 137-143, 138-144, 139-145, 140-146, 141-147, 142-148, 143-149, 144-150, 145-151, 146-152, 147-153, 148-154, 149-155, 150-156, 151-157, 152-158, 153-159, 154-160, 155-161, 156-162, 157-163, 158-164, 159-165, 160-166, 161-167, 162-168, 163-169, 164-170, 165-171, 166-172, 167-173, 168-174, 169-175, 170-176, 171-177, 172-178, 173-179, 174-180, 175-181, 176-182, 177-183, 178-184, 179-185, 180-186, 181-187, 182-188, 183-189, 184-190, 185-191, and 186-192 of SEQ ID NO:17. Corresponding fragments of a variant mouse CD20 polypeptide at least 70%, 75%, 80%, 85%, 90%, or 95% identical to any of these amino acids regions are also contemplated.

As known in the art, “sequence identity” between two polypeptides is determined by comparing the amino acid sequence of one polypeptide to the sequence of a second polypeptide. When discussed herein, whether any particular polypeptide is at least about 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

In other embodiments, the present invention includes an antibody, or antigen-binding fragment, variant, or derivative thereof which specifically or preferentially binds to at least one epitope of mouse CD20, where the epitope comprises, consists essentially of, or consists of at least about four to five amino acids of SEQ ID NO:17, at least seven, at least nine, or between at least about 15 to about 30 amino acids of SEQ ID NO:17. The amino acids of a given epitope of SEQ ID NO:17 as described may be, but need not be contiguous or linear. In certain embodiments, the at least one epitope of mouse CD20 comprises, consists essentially of, or consists of a non-linear epitope formed by the extracellular domain of mouse CD20 as expressed on the surface of a cell or as a soluble fragment, e.g., fused to an IgG Fc region. Thus, in certain embodiments the at least one epitope of mouse CD20 comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of SEQ ID NO:17, where non-contiguous amino acids form an epitope through protein folding.

In other embodiments, the present invention includes an antibody, or antigen-binding fragment, variant, or derivative thereof which specifically or preferentially binds to at least one epitope of mouse CD20, where the epitope comprises, consists essentially of, or consists of, in addition to one, two, three, four, five, six or more contiguous or non-contiguous amino acids of SEQ ID NO:17 as described above, and an additional moiety which modifies the protein, e.g., a carbohydrate moiety may be included such that the anti-mouse CD20 antibody binds with higher affinity to modified target protein than it does to an unmodified version of the protein. Alternatively, the anti-mouse CD20 antibody does not bind the unmodified version of the target protein at all.

In certain embodiments, an antibody, or antigen-binding fragment, variant, or derivative thereof of the invention binds specifically to at least one epitope of mouse CD20 or fragment or variant described above, i.e., binds to such an epitope more readily than it would bind to an unrelated, or random epitope; binds preferentially to at least one epitope of mouse CD20 or fragment or variant described above, i.e., binds to such an epitope more readily than it would bind to a related, similar, homologous, or analogous epitope; competitively inhibits binding of a reference antibody which itself binds specifically or preferentially to a certain epitope of mouse CD20 or fragment or variant described above; or binds to at least one epitope of mouse CD20 or fragment or variant described above with an affinity characterized by a dissociation constant K_{D} of less than about 5×10⁻² M, about 10⁻² M, about 5×10⁻³ M, about 10⁻³ M, about 5×10⁻⁴ M, about 10⁻⁴ M, about 5×10⁻⁵ M, about 10-5 M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10⁻¹⁰ M, about 10⁻¹⁰ M, about 5×10⁻¹¹ M, about 10⁻¹¹ M, about 5×10⁻¹² M, about 10⁻¹² M, about 5×10⁻¹³M, about 10⁻¹³ M, about 5×10⁻¹⁴ M, about 10⁻¹⁴ M, about 5×10⁻¹⁵ M, or about 10⁻¹⁵ M. In a particular aspect, the antibody or fragment thereof preferentially binds to a mouse CD20 polypeptide or fragment thereof, relative to a human CD20 polypeptide or fragment thereof.

As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” might include, for example, from 0.05 M to 0.005 M.

In specific embodiments, an antibody, or antigen-binding fragment, variant, or derivative thereof of the invention binds mouse CD20 polypeptides or fragments or variants thereof with an off rate (k(off)) of less than or equal to 5×10⁻² sec⁻¹, 10⁻² sec⁻, 5×10³ sec⁻¹ or 10⁻³ sec⁻¹. Alternatively, an antibody, or antigen-binding fragment, variant, or derivative thereof of the invention binds mouse CD20 polypeptides or fragments or variants thereof with an off rate (k(off)) of less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

In other embodiments, an antibody, or antigen-binding fragment, variant, or derivative thereof of the invention binds mouse CD20 polypeptides or fragments or variants thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. Alternatively, an antibody, or antigen-binding fragment, variant, or derivative thereof of the invention binds mouse CD20 polypeptides or fragments or variants thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×106 M⁻¹ sec⁻¹ or 10⁷ M sec⁻¹.

In various embodiments, an anti-mouse CD20 antibody, or antigen-binding fragment, variant, or derivative thereof as described herein, depletes B-cell populations in a non-human (e.g., mouse) subject. In a particular embodiment, the depletion is cause by induction of apoptosis by binding of the anti-mouse CD20 antibody to mouse CD20 expressed on B-cells.

Unless it is specifically noted, as used herein a “fragment thereof” in reference to an antibody refers to an antigen-binding fragment, i.e., a portion of the antibody which specifically binds to the antigen. In one embodiment, an antibody of the invention, e.g., an anti-mouse CD20 antibody, is a bispecific antibody or binding polypeptide, e.g., a bispecific antibody, minibody, domain deleted antibody, or fusion protein having binding specificity for more than one epitope, e.g., more than one antigen or more than one epitope on the same antigen. In one embodiment, a bispecific anti-mouse CD20 antibody or binding polypeptide has at least one binding domain specific for at least one epitope on a target polypeptide disclosed herein, e.g., mouse CD20. In another embodiment, a bispecific anti-mouse CD20 antibody, binding polypeptide, or antibody has at least one binding domain specific for an epitope on a target polypeptide and at least one target binding domain specific for a drug or toxin. In yet another embodiment, a bispecific anti-mouse CD20 antibody or binding polypeptide has at least one binding domain specific for an epitope on a target polypeptide disclosed herein, and at least one binding domain specific for a prodrug. A bispecific anti-mouse CD20 antibody or binding polypeptide may be a tetravalent antibody that has two target binding domains specific for an epitope of a target polypeptide disclosed herein and two target binding domains specific for a second target. Thus, a tetravalent bispecific anti-mouse CD20 antibody or binding polypeptide may be bivalent for each specificity.

Anti-mouse CD20 antibodies, or antigen-binding fragments, variants, or derivatives thereof of the invention, as known by those of ordinary skill in the art, can comprise a constant region which mediates one or more effector functions. For example, binding of the C1 component of complement to an antibody constant region may activate the complement system. Activation of complement is important in the opsonisation and lysis of cell pathogens. The activation of complement also stimulates the inflammatory response and may also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

Accordingly, certain embodiments of the invention include an anti-mouse CD20 antibody, or antigen-binding fragment, variant, or derivative thereof, in which at least a fraction of one or more of the constant region domains has been modified, deleted or otherwise altered so as to provide desired biochemical characteristics such as increased or reduced effector functions, increased ADCC or complement-dependent cytotoxicty, the ability to non-covalently dimerize, increased ability to localize at the site of a tumor, reduced serum half-life, or increased serum half-life when compared with a whole, unaltered antibody of approximately the same immunogenicity. For example, certain antibodies for use in the methods of B-cell depletion or screening described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the C_{H}2 domain will be deleted.

In certain anti-mouse CD20 antibodies, or antigen-binding fragments, variants, or derivatives thereof described herein, the Fc portion may be mutated to increase effector function using techniques known in the art. In other anti-mouse CD20 antibodies, or antigen-binding fragments, variants, or derivatives thereof described herein, the Fc portion may be mutated to decrease effector function using techniques known in the art. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, may easily be measured and quantified using well know immunological techniques without undue experimentation.

In other aspects, anti-mouse CD20 antibodies, or antigen-binding fragments, variants, or derivatives thereof of the invention can be used to study, delineate, or otherwise characterize the mechanism of effector function that leads to B-cell depletion by administering to transgenic mice and/or knockouts for FcγRI, FcγRII FcγRIII, or FcγRIV. In some embodiments, the mice may be transgenic for one or more human FcRs.

Modified forms of anti-mouse CD20 antibodies, or antigen-binding fragments, variants, or derivatives thereof of the invention can be made from whole precursor or parent antibodies using techniques known in the art. Exemplary techniques are discussed in more detail herein.

Anti-mouse CD20 antibodies, or antigen-binding fragments, variants, or derivatives thereof of the invention can be made or manufactured using techniques that are known in the art. In certain embodiments, antibody molecules or fragments thereof are “recombinantly produced,” i.e., are produced using recombinant DNA technology. Exemplary techniques for making antibody molecules or fragments thereof are discussed in more detail elsewhere herein.

Anti-mouse CD20 antibodies, or antigen-binding fragments, variants, or derivatives thereof of the invention also include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using a wide variety of techniques known in the art including the use of hybridoma and recombinant and phage display technology.

Using art recognized protocols, in one example, antibodies are raised in mammals by multiple subcutaneous or intraperitoneal injections of the relevant antigen (e.g., purified tumor associated antigens such as mouse CD20 or cells or cellular extracts comprising such antigens) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies (MAbs). Preferably, the lymphocytes are obtained from the spleen.

In this well known process, the relatively short-lived, or mortal, lymphocytes from a mammal which has been injected with antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by in vitro assays such as immunoprecipitation, radioimmunoassay (RIA), enzyme-linked immunoabsorbent assay (ELISA), or flow cytometry. After hybridoma cells are identified that produce antibodies of the desired specificity, affinity and/or activity, the clones may be subcloned by limiting dilution procedures and grown by standard methods. It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

Antibody fragments that recognize specific epitopes may be generated by known techniques. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain.

Examples of techniques which can be used to produce single-chain Fvs and antibodies include those described in U.S. Pat. Nos. 4,946,778 and 5,258,498.

In another embodiment, DNA encoding desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be synthetic as described herein) may be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

In one embodiment, an anti-mouse CD20 antibody of the invention comprises at least one heavy or light chain CDR of an antibody molecule. In another embodiment, an anti-mouse CD20 antibody of the invention comprises at least two CDRs from one or more antibody molecules. In another embodiment, an anti-mouse CD20 antibody of the invention comprises at least three CDRs from one or more antibody molecules. In another embodiment, an anti-mouse CD20 antibody of the invention comprises at least four CDRs from one or more antibody molecules. In another embodiment, an anti-mouse CD20 antibody of the invention comprises at least five CDRs from one or more antibody molecules. In another embodiment, an anti-mouse CD20 antibody of the invention comprises at least six CDRs from one or more antibody molecules. In particular embodiments, an anti-mouse CD20 antibody or antigen binding fragment or variant or derivative thereof of the invention comprises one, two, three, four, five or six CDRs of the 18B12 antibody VH or VL regions, which can be in any combination (e.g., one of the heavy chain CDRs and one of the light chain CDRs, two of the heavy chain CDRs, etc.). In another aspect, the anti-mouse CD20 antibodies of the present invention may comprise those residues from one or more CDRs, in particular, the 18B12 CDRs, that interact with the target polypeptide. One of ordinary skill in the art would be able to determine through routine methods which residues make contact and/or interact with the target polypeptide. The invention is also directed to methods of making such antibodies, or an antigen binding fragments, variants or derivatives thereof, and the use of same in animal models of disease, e.g., to observe effects of administration on the disease model, to deplete B-cells in the animal model of disease, and/or to test the compositions of the present invention or combinations of therapeutic agents with the compositions of the present invention for their ability to deplete B-cells and/or treat a disease or disorder in an animal model of disease.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within different framework regions. The framework regions may be naturally occurring or consensus framework regions, e.g., from mouse or from a different species. See, e.g., Kabat et al., U.S. Dept. of Health and Human Services, “Sequences of Proteins of Immunological Interest” (1983). Preferably, the polynucleotide generated by the combination of the framework regions and CDRs encodes an antibody that specifically binds to at least one epitope of a desired polypeptide, e.g., mouse CD20. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

Antibodies for use in the methods of the invention disclosed herein can be produced by any method known in the art for the synthesis of antibodies, in particular, by chemical synthesis or preferably, by recombinant expression techniques as described herein.

In one embodiment, an anti-mouse CD20 antibody, or antigen-binding fragment, variant, or derivative thereof of the invention comprises a synthetic constant region wherein one or more domains are partially or entirely deleted (“domain-deleted antibodies”). In certain embodiments compatible modified antibodies will comprise domain deleted constructs or variants wherein the entire C_{H}2 domain has been removed (ΔC_{H}2 constructs). For other embodiments a short connecting peptide may be substituted for the deleted domain to provide flexibility and freedom of movement for the variable region. Those skilled in the art will appreciate that such constructs are particularly preferred due to the regulatory properties of the C_{H}2 domain on the catabolic rate of the antibody. Domain deleted constructs can be derived using a vector (e.g., from Biogen Idec Incorporated) encoding an IgG, human constant domain (see, e.g., WO 02/060955A2 and WO02/096948A2). This exemplary vector was engineered to delete the C_{H}2 domain and provide a synthetic vector expressing a domain deleted IgG₁ constant region.

In certain embodiments, anti-mouse CD20 antibodies, or antigen-binding fragments, variants, or derivatives thereof of the invention are minibodies. Minibodies can be made using methods described in the art (see, e.g., US Pat. No. 5,837,821 or WO 94/09817A1).

Certain embodiments comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

The present invention also provides antibodies that comprise, consist essentially of, or consist of, variants (including derivatives) of antibody molecules (e.g., the V_{H} regions and/or V_{L} regions) described herein, which antibodies or fragments thereof immunospecifically bind to a mouse CD20 polypeptide or fragment or variant thereof. Standard techniques known to those of skill in the art can be used to introduce mutations in the nucleotide sequence encoding an anti-mouse CD20 antibody, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference V^{H} region, V_{H}CDR1, V_{H}CDR2, V_{H}CDR3, V_{L} region, V_{L}CDR1, V_{L}CDR2, or V_{L}CDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind a mouse CD20 polypeptide).

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve a hybridoma's antibody production. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind at least one epitope of a mouse CD20 polypeptide) can be determined using techniques described herein or by routinely modifying techniques known in the art.

Polynucleotides Encoding Anti-mouse CD20 Antibodies

The present invention also provides for nucleic acid molecules encoding anti-mouse CD20 antibodies, or antigen-binding fragments, variants, or derivatives thereof of the invention.

In one embodiment, the present invention provides an isolated polynucleotide comprising, consisting essentially of, or consisting of a nucleic acid encoding an immunoglobulin heavy chain variable region (VH), where the CDR1, CDR2, and CDR3 regions of the VH are at least 80%, 85%, 90% or 95% identical to reference heavy chain CDR1, CDR2, and CDR3 amino acid sequences from the 18B12 antibody disclosed herein. Thus, according to this embodiment a heavy chain variable region of the invention may have CDR1, CDR2, and CDR3 polypeptide sequences related to the groups shown in Table 2:

TABLE 2

VH CDR1, CDR2, AND CDR3 AMINO ACID AND NUCLEO-
TIDE REFERENCE SEQUENCES FROM 18B12*

| | | |
|---|---|---|
| | | SEQ |
| | Amino Acid Sequence | ID |
| CDR | Nucleotide Sequence | NO |

| | | |
|---|---|---|
| VH | SYWMH | 14 |
| CDR1 | AGCTACTGGATGCAC | 8 |
| VH | VIDPSDNYTKYNQKFKG | 15 |
| CDR2 | GTGATTGATCCTTCTGATAATTATACTAAGTACA | 9 |
| | ATCAAAAGTTTAAGGGC | |
| VH | EGYYGSSPWFAY | 16 |
| CDR3 | GAGGGCTACTACGGTAGTAGTCCCTGGTTTGCTTAC | 10 |

*Determined by the Kabat system (see supra).

According to this aspect of the invention, an antibody or antigen-binding fragment comprising the VH encoded by the polynucleotide may specifically bind to mouse CD20.

In another embodiment, the present invention provides an isolated polynucleotide comprising, consisting essentially of, or consisting of a nucleic acid encoding an immunoglobulin heavy chain variable region (VH) in which the CDR1, CDR2, and CDR3 regions have polypeptide sequences which are identical to the CDR1, CDR2, and CDR3 groups shown in Table 2. According to this aspect of the invention, an antibody or antigen-binding fragment comprising the VH encoded by the polynucleotide may specifically bind to mouse CD20.

In a further aspect, the present invention provides an isolated polynucleotide comprising, consisting essentially of, or consisting of a nucleic acid encoding an immunoglobulin heavy chain variable region (VH) in which the CDR1, CDR2, and CDR3 regions are encoded by nucleotide sequences which are identical to the nucleotide sequences which encode the CDR1, CDR2, and CDR3 groups shown in Table 2. According to this aspect of the invention, an antibody or antigen-binding fragment comprising the VH encoded by the polynucleotide may specifically bind to mouse CD20.

In a further embodiment, the present invention includes an isolated polynucleotide comprising, consisting essentially of, or consisting of a nucleic acid encoding a VH at least 80%, 85%, 90% 95% or 100% identical to a reference VH polypeptide of SEQ ID NO:4. According to this aspect of the invention, an antibody or antigen-binding fragment comprising the VH encoded by the polynucleotide may specifically bind to mouse CD20.

In another aspect, the present invention includes an isolated polynucleotide comprising, consisting essentially of, or consisting of a nucleic acid sequence encoding a VH of SEQ ID NO:2. According to this aspect of the invention, an antibody or antigen-binding fragment comprising the VH encoded by the polynucleotide may specifically bind to mouse CD20.

In certain embodiments it is contemplated that an antibody or antigen-binding fragment thereof comprising, consisting essentially of, or consisting of a VH which is encoded by one or more of the polynucleotides described above will specifically bind to the same epitope as the 18B12 monoclonal antibody, or will competitively inhibit such a monoclonal antibody from binding to mouse CD20.

In certain embodiments it is further contemplated that an antibody or antigen-binding fragment thereof comprising, consisting essentially of, or consisting of a VH which is encoded by one or more of the polynucleotides described above will specifically bind to a mouse CD20 polypeptide or fragment thereof, or a mouse CD20 variant polypeptide, with an affinity characterized by a dissociation constant (K_{D}) no greater than 5×10⁻² M, 10⁻² M, 5×10⁻³ M, 10⁻³ M, 5×10⁻⁴M 10⁻⁴ M, 5×10⁻⁵ M, 10-⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In another embodiment, the present invention provides an isolated polynucleotide comprising, consisting essentially of, or consisting of a nucleic acid encoding an immunoglobulin light chain variable region (VL), where the CDR1, CDR2, and CDR3 regions of the VL are at least 80%, 85%, 90% or 95% identical to reference heavy chain CDR1, CDR2, and CDR3 amino acid sequences from monoclonal anti-mouse CD20 antibodies disclosed herein. Thus, according to this embodiment a heavy chain variable region of the invention may have CDR1, CDR2, and CDR3 polypeptide sequences related to the groups shown in Table 3:

TABLE 3

VL CDR1, CDR2, AND CDR3 AMINO ACID AND NUCLEO-
TIDE REFERENCE SEQUENCES FROM 18B12*

| | | |
|---|---|---|
| | | SEQ |
| | Amino Acid Sequence | ID |
| CDR | Nucleotide Sequence | NO |

| | | |
|---|---|---|
| VL | RARSSVVSYIH | 11 |
| CDR1 | AGGGCCAGGTCAAGTGTGAGTTACATACAC | 5 |
| VL | ATSNLAS | 12 |
| CDR2 | GCCACATCCAACCTGGCTTCT | 6 |
| VL | QQWSSKPPT | 13 |
| CDR3 | CAGCAGTGGAGTAGTAAGCCACCCACG | 7 |

*Determined by the Kabat system (see supra).

According to this aspect of the invention, an antibody or antigen-binding fragment comprising the VL encoded by the polynucleotide may specifically bind to mouse CD20.

In another embodiment, the present invention provides an isolated polynucleotide comprising, consisting essentially of, or consisting of a nucleic acid encoding an immunoglobulin light chain variable region (VL) in which the CDR1, CDR2, and CDR3 regions have polypeptide sequences which are identical to the CDR1, CDR2, and CDR3 groups shown in Table 3. According to this aspect of the invention, an antibody or antigen-binding fragment comprising the VL encoded by the polynucleotide may specifically bind to mouse CD20.

In a further aspect, the present invention provides an isolated polynucleotide comprising, consisting essentially of, or consisting of a nucleic acid encoding an immunoglobulin light chain variable region (VL) in which the CDR1, CDR2, and CDR3 regions are encoded by nucleotide sequences which are identical to the nucleotide sequences which encode the CDR1, CDR2, and CDR3 groups shown in Table 3. According to this aspect of the invention, an antibody or antigen-binding fragment comprising the VL encoded by the polynucleotide may specifically bind to mouse CD20.

In a further embodiment, the present invention includes an isolated polynucleotide comprising, consisting essentially of, or consisting of a nucleic acid encoding a VL at least 80%, 85%, 90%, 95%, or 100% identical to a reference VL polypeptide sequence of SEQ ID NO:3 or SEQ ID NO:33. According to this aspect of the invention, an antibody or antigen-binding fragment comprising the VL encoded by the polynucleotide may specifically bind to mouse CD20.

In another aspect, the present invention includes an isolated polynucleotide comprising, consisting essentially of, or consisting of a nucleic acid sequence encoding a VL of SEQ ID NO:1 or SEQ ID NO:32. According to this aspect of the invention, an antibody or antigen-binding fragment comprising the VL encoded by the polynucleotide may specifically bind to mouse CD20.

In certain embodiments it is contemplated that an antibody or antigen-binding fragment thereof comprising, consisting essentially of, or consisting of a VL which is encoded by one or more of the polynucleotides described above will specifically bind to the same epitope as the 18B12 monoclonal antibody, or will competitively inhibit such a monoclonal antibody from binding to mouse CD20.

In certain embodiments it is further contemplated that an antibody or antigen-binding fragment thereof comprising, consisting essentially of, or consisting of a VL which is encoded by one or more of the polynucleotides described above will specifically bind to a mouse CD20 polypeptide or fragment thereof, or a mouse CD20 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 533 10⁻¹³ M, 10⁻¹³ M, 5×10⁻¹⁴ M, 10⁻¹⁴ M, 5×10⁻¹⁵ M, or 10⁻¹⁵ M.

Any of the polynucleotides described above may further include additional nucleic acids, encoding, e.g., a signal peptide to direct secretion of the encoded polypeptide, antibody constant regions as described herein, or other heterologous polypeptides as described herein.

Also, as described in more detail elsewhere herein, the present invention includes compositions comprising the polynucleotides comprising one or more of the polynucleotides described above.

The polynucleotides may be produced or manufactured by any method known in the art. For example, if the nucleotide sequence of the antibody is known, a polynucleotide encoding the antibody may be assembled from chemically synthesized oligonucleotides, which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an anti-mouse CD20 antibody, or antigen-binding fragment, variant, or derivative thereof may be generated from nucleic acid from a suitable source. If a clone containing a nucleic acid encoding a particular antibody is not available, but the sequence of the antibody molecule is known, a nucleic acid encoding the antibody may be chemically synthesized or obtained from a suitable source (e.g., an antibody cDNA library, or a cDNA library generated from, or nucleic acid, preferably poly A+RNA, isolated from, any tissue or cells expressing the antibody or other anti-mouse CD20 antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody or other anti-mouse CD20 antibody. Amplified nucleic acids generated by PCR may then be cloned into replicable cloning vectors using any method well known in the art.

Once the nucleotide sequence and corresponding amino acid sequence of the anti-mouse CD20 antibody, or antigen-binding fragment, variant, or derivative thereof is determined, its nucleotide sequence may be manipulated using methods well known in the art for the manipulation of nucleotide sequences, e.g., recombinant DNA techniques, site directed mutagenesis, PCR, etc., to generate antibodies having a different amino acid sequence, for example to create amino acid substitutions, deletions, and/or insertions.

A polynucleotide encoding a anti-mouse CD20 antibody, or antigen-binding fragment, variant, or derivative thereof can be composed of any polyribonucleotide or polydeoxribonucleotide, which may be unmodified RNA or DNA or modified RNA or DNA. For example, a polynucleotide encoding an anti-mouse CD20 antibody, or antigen-binding fragment, variant, or derivative thereof can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding an anti-mouse CD20 antibody, or antigen-binding fragment, variant, or derivative thereof can be composed of triple-stranded regions comprising RNA or DNA or both RNA and DNA. A polynucleotide encoding an anti-mouse CD20 antibody, or antigen-binding fragment, variant, or derivative thereof may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide”embraces chemically, enzymatically, or metabolically modified forms.

An isolated polynucleotide encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

Anti-mouse CD20 Antibody Polypeptides

The present invention is further directed to isolated polypeptides which make up anti-mouse CD20 antibodies, and polynucleotides encoding such polypeptides. Anti-mouse CD20 antibodies of the present invention comprise polypeptides, e.g., amino acid sequences encoding mouse CD20-specific antigen binding regions derived from immunoglobulin molecules. A polypeptide or amino acid sequence “derived from” a designated protein refers to the origin of the polypeptide. In certain cases, the polypeptide or amino acid sequence which is derived from a particular starting polypeptide or amino acid sequence has an amino acid sequence that is essentially identical to that of the starting sequence, or a portion thereof, wherein the portion consists of at least 10-20 amino acids, at least 20-30 amino acids, at least 30-50 amino acids, or which is otherwise identifiable to one of ordinary skill in the art as having its origin in the starting sequence.

In one embodiment, the present invention provides an isolated polypeptide comprising, consisting essentially of, or consisting of an immunoglobulin heavy chain variable region (VH), where the CDR1, CDR2, and CDR3 regions of the VU are at least 80%, 85%, 90% or 95% identical to reference heavy chain CDR1, CDR2, and CDR3 amino acid sequences from monoclonal anti-mouse CD20 antibodies (e.g., 18B12) disclosed herein. Thus, according to this embodiment a heavy chain variable region of the invention may have CDR1, CDR2, and CDR3 polypeptide sequences as shown in Table 2, supra. According to this aspect of the invention, an antibody or antigen-binding fragment comprising the VH may specifically bind to mouse CD20.

In another embodiment, the present invention provides an isolated polypeptide comprising, consisting essentially of, or consisting of an immunoglobulin heavy chain variable region (VH) in which the CDR1, CDR2, and CDR3 regions have polypeptide sequences which are identical to the CDR1, CDR2, and CDR3 sequences shown in Table 2. According to this aspect of the invention, an antibody or antigen-binding fragment comprising the VH may specifically bind to mouse CD20.

In a further embodiment, the present invention includes an isolated polypeptide comprising, consisting essentially of, or consisting of a VH at least 80%, 85%, 90% 95% or 100% identical to a reference VH polypeptide sequence of SEQ ID NO:4. According to this aspect of the invention, an antibody or antigen-binding fragment comprising the VH may specifically bind to mouse CD20.

In certain embodiments it is contemplated that an antibody or antigen-binding fragment thereof comprising, consisting essentially of, or consisting of a VH described above will specifically bind to the same epitope as the 18B12 monoclonal antibody, or will competitively inhibit such a monoclonal antibody from binding to mouse CD20.

In certain embodiments it is further contemplated that an antibody or antigen-binding fragment thereof comprising, consisting essentially of, or consisting of a VH described above will specifically bind to a mouse CD20 polypeptide or fragment thereof, or a mouse CD20 variant polypeptide, with an affinity characterized by a dissociation constant (K_{D}) no greater than 5×10⁻² M, 10⁻² M, 5×10⁻³ M, 10-³ M, 5×10⁻⁴ M, 10⁻⁴ M, 5×10⁻⁵ M, 10⁻⁶ M, 5×10⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10^{—}M, 10⁻¹³ M, 5×10⁻¹⁴ M, 10⁻¹⁴ M, 5×10⁻¹⁵ M, or 10⁻¹⁵ M.

In another embodiment, the present invention provides an isolated polypeptide comprising, consisting essentially of, or consisting of an immunoglobulin light chain variable region (VL), where the CDR1, CDR2, and CDR3 regions of the VL are at least 80%, 85%, 90% or 95% identical to reference heavy chain CDR1, CDR2, and CDR3 amino acid sequences from monoclonal anti-mouse CD20 antibodies (e.g., 18B12) disclosed herein. Thus, according to this embodiment a heavy chain variable region of the invention may have CDR1, CDR2, and CDR3 polypeptide sequences as shown in Table 3, supra. According to this aspect of the invention, an antibody or antigen-binding fragment comprising the VL may specifically bind to mouse CD20.

In another embodiment, the present invention provides an isolated polypeptide comprising, consisting essentially of, or consisting of an immunoglobulin light chain variable region (VL) in which the CDR1, CDR2, and CDR3 regions have polypeptide sequences which are identical to the CDR1, CDR2, and CDR3 groups shown in Table 3. According to this aspect of the invention, an antibody or antigen-binding fragment comprising the VL may specifically bind to mouse CD20.

In a further embodiment, the present invention includes an isolated polypeptide comprising, consisting essentially of, or consisting of a VL at least 80%, 85%, 90%, 95%, or 100% identical to a reference VL polypeptide sequence of SEQ ID NO:3 or SEQ ID NO:33. According to this aspect of the invention, an antibody or antigen-binding fragment comprising the VL encoded by the polynucleotide may specifically bind to mouse CD20.

In certain embodiments it is contemplated that an antibody or antigen-binding fragment thereof comprising, consisting essentially of, or consisting of a VL described above will specifically bind to the same epitope as the 18B12 monoclonal antibody, or will competitively inhibit such a monoclonal antibody from binding to mouse CD20.

In certain embodiments it is further contemplated that an antibody or antigen-binding fragment thereof comprising, consisting essentially of, or consisting of a VL described above will specifically bind to a mouse CD20 polypeptide or fragment thereof, or a mouse CD20 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10^{31 8} M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Any of the polypeptides described above may further include additional polypeptides, e.g., a signal peptide to direct secretion of the encoded polypeptide, antibody constant regions as described herein, or other heterologous polypeptides as described herein.

Also, as described in more detail elsewhere herein, the present invention includes compositions comprising the polypeptides described above.

It will also be understood by one of ordinary skill in the art that anti-mouse CD20 antibody polypeptides as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, or 95% identical to the starting sequence.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In certain embodiments, a polypeptide or amino acid sequence derived from a designated protein may have one to five, one to ten, one to fifteen, or one to twenty individual amino acid substitutions, insertions, or deletions relative to the starting sequence.

In certain embodiments, an anti-mouse CD20 antibody polypeptide comprises an amino acid sequence or one or more moieties not normally associated with an antibody. Exemplary modifications are described in more detail below. For example, a single-chain fv antibody fragment of the invention may comprise a flexible linker sequence, or may be modified to add a functional moiety (e.g., PEG, a drug, a toxin, or a label).

An anti-mouse CD20 antibody polypeptide of the invention may comprise, consist essentially of, or consist of a fusion protein. Fusion proteins are chimeric antibody molecules which comprise, for example, an immunoglobulin antigen-binding domain with at least one target binding site, and at least one heterologous portion, i.e., a portion with which it is not naturally linked in nature. The amino acid sequences may normally exist in separate proteins that are brought together in the fusion polypeptide or they may normally exist in the same protein but are placed in a new arrangement in the fusion polypeptide. Fusion proteins may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a distinct entity from that of the rest of the entity to which it is being compared. For instance, as used herein, a “heterologous polypeptide” to be fused to an anti-mouse CD20 antibody, or an antigen-binding fragment, variant, or analog thereof is derived from a non-immunoglobulin polypeptide of the same species, or an immunoglobulin or non-immunoglobulin polypeptide of a different species.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Alternatively, in another embodiment, mutations may be introduced randomly along all or part of the immunoglobulin coding sequence, such as by saturation mutagenesis, and the resultant mutants can be incorporated into anti-mouse CD20 antibodies for use in the methods disclosed herein and screened for their ability to bind to the desired antigen, e.g., mouse CD20.

Fusion Proteins and Antibody Conjugates

As discussed in more detail elsewhere herein, anti-mouse CD20 antibodies, or antigen-binding fragments, variants, or derivatives thereof of the invention may further be recombinantly fused to a heterologous polypeptide at the N- or C-terminus or chemically conjugated (including covalent and non-covalent conjugations) to polypeptides or other compositions. For example, mouse CD20-specific antibodies may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

Anti-mouse CD20 antibodies, or antigen-binding fragments, variants, or derivatives thereof of the invention include derivatives that are modified, i.e., by the covalent attachment of any type of molecule to the antibody such that covalent attachment does not prevent the antibody binding mouse CD20. For example, but not by way of limitation, the antibody derivatives include antibodies that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Anti-mouse CD20 antibodies, or antigen-binding fragments, variants, or derivatives thereof of the invention can be composed of amino acids joined to each other by peptide bonds or modified peptide bonds, i.e., peptide isosteres, and may contain amino acids other than the 20 gene-encoded amino acids. Mouse CD20-specfic antibodies may be modified by natural processes, such as posttranslational processing, or by chemical modification techniques which are well known in the art. Such modifications are well described in basic texts and in more detailed monographs, as well as in a voluminous research literature. Modifications can occur anywhere in the mouse CD20-specific antibody, including the peptide backbone, the amino acid side-chains and the amino or carboxyl termini, or on moieties such as carbohydrates. It will be appreciated that the same type of modification may be present in the same or varying degrees at several sites in a given mouse CD20-specific antibody. Also, a given mouse CD20-specific antibody may contain many types of modifications. Mouse CD20-specific antibodies may be branched, for example, as a result of ubiquitination, and they may be cyclic, with or without branching. Cyclic, branched, and branched cyclic mouse CD20-specific antibodies may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination.

The present invention also provides for fusion proteins comprising an anti-mouse CD20 antibody, or antigen-binding fragment, variant, or derivative thereof, and a heterologous polypeptide. The heterologous polypeptide to which the antibody is fused may be useful for function or is useful to target the mouse CD20 polypeptide expressing cells. In one embodiment, a fusion protein of the invention comprises, consists essentially of, or consists of, a polypeptide having the amino acid sequence of any one or more of the V_{H} regions of an antibody of the invention or the amino acid sequence of any one or more of the V_{L} regions of an antibody of the invention or fragments or variants thereof, and a heterologous polypeptide sequence. In another embodiment, a fusion protein for use in the methods of using anti-mouse CD20 antibodies disclosed herein comprises, consists essentially of, or consists of a polypeptide having the amino acid sequence of any one, two, three of the V_{H} CDRs of a mouse CD20-specific antibody, or fragments, variants, or derivatives thereof, or the amino acid sequence of any one, two, three of the V_{L} CDRs of a mouse CD20-specific antibody, or fragments, variants, or derivatives thereof, and a heterologous polypeptide sequence. In one embodiment, the fusion protein comprises a polypeptide having the amino acid sequence of a V_{H} CDR3 of a mouse CD20-specific antibody of the present invention, or fragment, derivative, or variant thereof, and a heterologous polypeptide sequence, which fusion protein specifically binds to at least one epitope of mouse CD20. In another embodiment, a fusion protein comprises a polypeptide having the amino acid sequence of at least one V_{H} region of a mouse CD20-specific antibody of the invention and the amino acid sequence of at least one V_{L} region of a mouse CD20-specific antibody of the invention or fragments, derivatives or variants thereof, and a heterologous polypeptide sequence. Preferably, the V_{H} and V_{L} regions of the fusion protein correspond to a single source antibody (or scFv or Fab fragment) which specifically binds at least one epitope of mouse CD20. In yet another embodiment, a fusion protein for use in the diagnostic and treatment methods disclosed herein comprises a polypeptide having the amino acid sequence of any one, two, three or more of the V_{H} CDRs of a mouse CD20-specific antibody and the amino acid sequence of any one, two, three or more of the V_{L} CDRs of a mouse CD20-specific antibody, or fragments or variants thereof, and a heterologous polypeptide sequence. Preferably, two, three, four, five, six, or more of the V_{H}CDR(s) or V_{L}CDR(s) correspond to single source antibody (or scFv or Fab fragment) of the invention. Nucleic acid molecules encoding these fusion proteins are also encompassed by the invention.

Exemplary fusion proteins include fusions of the T cell receptor; CD4; L-selectin (homing receptor); CD28 and B7; CTLA-4; CD22; TNF receptor; and IgE receptor a.

As discussed elsewhere herein, anti-mouse CD20 antibodies, or antigen-binding fragments, variants, or derivatives thereof of the invention may be fused to heterologous polypeptides to increase the in vivo half life of the polypeptides or for use in immunoassays using methods known in the art. For example, in one embodiment, PEG can be conjugated to the anti-mouse CD20 antibodies of the invention to increase their half-life in vivo.

Moreover, anti-mouse CD20 antibodies, or antigen-binding fragments, variants, or derivatives thereof of the invention can be fused to marker sequences, such as a peptide to facilitates their purification or detection. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. For instance, hexa-histidine provides for convenient purification of the fusion protein.

Other peptide tags useful for purification include, but are not limited to, the “HA” tag, which corresponds to an epitope derived from the influenza hemagglutinin protein, and the “flag” tag.

Fusion proteins can be prepared using methods that are well known in the art. The precise site at which the fusion is made may be selected empirically to optimize the secretion or binding characteristics of the fusion protein. DNA encoding the fusion protein is then transfected into a host cell for expression.

Anti-mouse CD20 antibodies of the present invention may be used in non-conjugated form or may be conjugated to at least one of a variety of molecules, e.g., to improve the potential therapeutic properties of the molecule, to facilitate target detection, or for imaging or therapy of the subject. Anti-mouse CD20 antibodies, or antigen-binding fragments, variants, or derivatives thereof of the invention can be labeled or conjugated either before or after purification, when purification is performed.

In particular, anti-mouse CD20 antibodies, or antigen-binding fragments, variants, or derivatives thereof of the invention may be conjugated to therapeutic agents, prodrugs, peptides, proteins, enzymes, viruses, lipids, biological response modifiers, pharmaceutical agents, or PEG.

Those skilled in the art will appreciate that conjugates may also be assembled using a variety of techniques depending on the selected agent to be conjugated. For example, conjugates with biotin are prepared e.g. by reacting a binding polypeptide with an activated ester of biotin such as the biotin N-hydroxysuccinimide ester. Similarly, conjugates with a fluorescent marker may be prepared in the presence of a coupling agent, e.g. those listed herein, or by reaction with an isothiocyanate, preferably fluorescein-isothiocyanate. Conjugates of the anti-mouse CD20 antibodies, or antigen-binding fragments, variants, or derivatives thereof of the invention are prepared in an analogous manner.

The present invention further encompasses anti-mouse CD20 antibodies, or antigen-binding fragments, variants, or derivatives thereof of the invention conjugated to a diagnostic or therapeutic agent. The anti-mouse CD20 antibodies can be used diagnostically to, for example, monitor the development or progression of a B-cell disease as part of a clinical testing procedure to, e.g., determine the efficacy of a given treatment and/or prevention regimen. Detection can be facilitated by coupling the anti-mouse CD20 antibody, or antigen-binding fragment, variant, or derivative thereof to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, β-galactosidase, or acetylcholinesterase; examples of suitable prosthetic group complexes include streptavidin/biotin and avidinibiotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, ¹¹¹In or ⁹⁹Tc.

A anti-mouse CD20 antibody, or antigen-binding fragment, variant, or derivative thereof also can be detectably labeled by coupling it to a chemiluminescent compound. The presence of the chemiluminescent-tagged anti-mouse CD20 antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which an anti-mouse CD20 antibody, or antigen-binding fragment, variant, or derivative thereof can be detectably labeled is by linking the same to an enzyme and using the linked product in an enzyme immunoassay (EIA). The enzyme, which is bound to the anti-mouse CD20 antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the anti-mouse CD20 antibody, or antigen-binding fragment, variant, or derivative thereof, it is possible to detect the antibody through the use of a radioimmunoassay (RIA). The radioactive isotope can be detected by means including, but not limited to, a gamma counter, a scintillation counter, or autoradiography.

An anti-mouse CD20 antibody, or antigen-binding fragment, variant, or derivative thereof can also be detectably labeled using fluorescence emitting metals such as 152^{Eu}, or others of the lanthanide series. These metals can be attached to the antibody using such metal chelating groups as diethylenetriaminepentacetic acid (DTPA) or ethylenediaminetetraacetic acid (EDTA).

Techniques for conjugating various moieties to an anti-mouse CD20 antibody, or antigen-binding fragment, variant, or derivative thereof are well known.

IgG2a Antibodies

In one aspect, the present invention is directed to antibodies or antigen binding fragments, variants, or derivatives thereof comprising a heavy chain constant region of the IgG2a isotype, and methods of using the antibodies. In a preferred embodiment, the antibodies are mouse IgG2a antibodies. In a preferred embodiment, the IgG2a antibodies are mouse IgG2a anti-mouse CD20 antibodies. In a particularly preferred embodiment, the mouse IgG2a antibodies comprise a heavy chain constant region encoded by a nucleotide sequence of SEQ ID NO:38. In another particularly preferred embodiment, the mouse IgG2a antibodies comprise a heavy chain constant region encoded by a polypeptide sequence of SEQ ID NO:39.

The IgG2a antibodies of the present invention preferably comprise a variable region or fragment thereof that comprises an antigen binding domain that specifically binds a target antigen. In a particular embodiment, the IgG2a antibodies are specific for a target antigen in a non-human animal, preferably a mouse. In some embodiments, the target antigen is a growth factor including, but not limited to, fibroblast growth factors (FGFs) (e.g., FGF1, FGF2, FGF4, FGF8), epidermal growth factors (EGFs) (e.g., EGF1, EGF2, EGF3), platelet-derived growth factors (PDGFs), vascular endothelial growth factors (VEGFs), nerve growth factors (NGFs), colony stimulating factors (CSFs), transforming growth factors (TGFs) (e.g., TGFβ, TGFα), bone morphogenetic proteins (BMPs) (e.g., BMP2, BMP4), tumor necrosis factors (TNFs) (e.g., TNFα), neurotrophins, insulin-like growth factors (IGFs) (e.g., IGF1, IGF2), and erythropoietin. In other embodiments, the target antigen is a receptor for a growth factor, including but not limited to receptors for any of the above-identified growth factors. In further embodiments, the target antigen is a cancer-associated antigen, including but not limited to MAGE proteins, BAGE proteins, GAGE proteins, p53, CEA, α-fetoprotein, HCG, PSA, TAG-72, and CA125. In a preferred embodiment, the target antigen is mouse CD20.

The IgG2a antibodies of the present invention may be produced and/or modified according to methods known in the art and/or as described herein (e.g., as described with respect to anti-mouse CD20 antibodies).

The IgG2a antibodies of the present invention can be administered to non-human animal models of human disease to simulate (e.g., model) the treatment of human disease with human IgG1 antibodies and/or to determine the effects of effector functions associated with (e.g., elicited or induced by) the IgG2a antibodies on the animal model of disease. The effects of administration of, for example, mouse IgG2a antibodies to a mouse model of human disease can be used, for example, to predict the effects of a human IgG1 antibody administered to a human with that disease or to determine the effect, degree, or other parameters of the effector functions associated with the IgG2a antibodies on the disease in the mouse model, etc. The IgG2a antibodies of the present invention can also be co-administered with other therapeutic agents to an animal model (e.g., a mouse model) of disease to determine the effects of the IgG2a antibodies in combination therapies.

In one embodiment, the IgG2a antibody of the present invention is a monoclonal antibody. In a further embodiment, the IgG2a antibody is engineered to be an IgG2a isotype antibody. For example, in one embodiment, a monoclonal antibody that was originally of a different isotype (e.g., IgG1, IgG2b, IgG2c, etc.) is engineered to replace the original heavy chain constant region with an IgG2a constant region. In a specific embodiment the IgG2a antibody is a mouse IgG2a antibody, and more specifically, a mouse IgG2a anti-mouse CD20 antibody. In a specific embodiment, the IgG2a constant region is of the “a” allotype. The IgG2a antibodies of the present invention (e.g., IgG2a anti-mouse CD20 antibodies) are engineered forms or fragments as described elsewhere herein (e.g. mutlispecific, scFV, Fab, multivalent, etc.).

Expression of Antibody Polypeptides

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art.

In one embodiment, cDNAs that encode the light and the heavy chains of the antibody may be made, either simultaneously or separately, using reverse transcriptase and DNA polymerase in accordance with well known methods. PCR may be initiated by consensus constant region primers or by more specific primers based on the published heavy and light chain DNA and amino acid sequences. As discussed above, PCR also may be used to isolate DNA clones encoding the antibody light and heavyi chains. In this case the libraries may be screened by consensus primers or larger homologous probes, such as mouse constant region probes.

DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis.

Following manipulation of the isolated genetic material to provide anti-mouse CD20 antibodies or mouse IgG2a anti-mouse CD20 antibodies, or antigen-binding fragments, variants, or derivatives thereof of the invention, the polynucleotides encoding the anti-mouse CD20 antibodies or mouse IgG2a antibodies are typically inserted in an expression vector for introduction into host cells that may be used to produce the desired quantity of anti-mouse CD20 antibody or mouse IgG2a antibody.

Recombinant expression of an antibody, or fragment, derivative or analog thereof, e.g., a heavy or light chain of an antibody which binds to a target molecule described herein, e.g., mouse CD20, requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain is advantageously placed before the heavy chain to avoid an excess of toxic free heavy chain. The coding sequences for the heavy and light chains may comprise cDNA or genomic DNA.

The term “vector” or “expression vector” is used herein to mean vectors used in accordance with the present invention as a vehicle for introducing into and expressing a desired gene in a host cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals.

In particularly preferred embodiments the cloned variable region genes are inserted into an expression vector along with the heavy and light chain constant region genes as discussed above. Any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). In general, screening large numbers of transformed cells for those which express suitably high levels of immunoglobulin heavy and light chains is routine experimentation which can be carried out, for example, by robotic systems. Vector systems are also taught in U.S. Pat. Nos. 5,736,137 and 5,658,570, each of which is incorporated by reference in its entirety herein. This system provides for high expression levels, e.g., >30 pg/cell/day. Other exemplary vector systems are disclosed e.g., in U.S. Pat. No. 6,413,777.

In other preferred embodiments the anti-mouse CD20 antibodies, or IgG2a antibodies, or antigen-binding fragments, variants, or derivatives thereof of the invention may be expressed using polycistronic constructs such as those disclosed in United States Patent Application Publication No. 2003-0157641 A1, filed Nov. 18, 2002, and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of, e.g., anti-mouse CD20 antibodies, e.g., binding polypeptides, e.g., mouse CD20-specific antibodies or immunospecific fragments thereof in eukaryotic host cells. Compatible IRES sequences are disclosed in U.S. Pat. No. 6,193,980 which is also incorporated herein. Those skilled in the art will appreciate that such expression systems may be used to effectively produce the full range of mouse CD20 antibodies disclosed in the instant application.

More generally, once the vector or DNA sequence encoding a monomeric subunit of the anti-mouse CD20 antibody (or mouse IgG2a antibody) has been prepared, the expression vector may be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. Typically, plasmid introduction into the host is via electroporation. The host cells harboring the expression construct are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

The expression vector is transferred to a host cell by conventional techniques and the transfected cells are then cultured by conventional techniques to produce an antibody for use in the methods described herein. Thus, the invention includes host cells containing a polynucleotide encoding an antibody of the invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed below.

As used herein, “host cells” refers to cells which harbor vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

A variety of host-expression vector systems may be utilized to express antibody molecules for use in the methods described herein. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies.

The host cell line used for protein expression is often of mammalian origin; those skilled in the art are credited with ability to preferentially determine particular host cell lines which are best suited for the desired gene product to be expressed therein. Exemplary host cell lines include, but are not limited to, CHO (Chinese Hamster Ovary), DG44 and DUXB 11 (Chinese Hamster Ovary lines, DHFR minus), HELA (human cervical carcinoma), CVI (monkey kidney line), COS (a derivative of CVI with SV40 T antigen), VERY, BHK (baby hamster kidney), MDCK, 293, WI38, R1610 (Chinese hamster fibroblast) BALBC/3T3 (mouse fibroblast), HAK (hamster kidney line), SP2/O (mouse myeloma), P3x63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the antibody molecule.

A number of selection systems may be used, including but not limited to the herpes simplex virus thymidine kinase, hypoxanthine-guanine phosphoribosyltransferase, and adenine phosphoribosyltransferase genes can be employed in tk-, hgprt- or aprt-cells, respectively. Also, antimetabolite resistance can be used as the basis of selection for the following genes: dhfr, which confers resistance to methotrexate; gpt, which confers resistance to mycophenolic acid; neo, which confers resistance to the aminoglycoside G-418; and hygro, which confers resistance to hygromycin.

The expression levels of an antibody molecule can be increased by vector amplification. When a marker in the vector system expressing antibody is amplifiable, increase in the level of inhibitor present in culture of host cell will increase the number of copies of the marker gene. Since the amplified region is associated with the antibody gene, production of the antibody will also increase.

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein.

Genes encoding anti-mouse CD20 antibodies or mouse IgG2a antibodies, or antigen-binding fragments, variants, or derivatives thereof of the invention can also be expressed non-mammalian cells such as bacteria or yeast or plant cells. Bacteria which readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologouspolypeptides must be isolated, purified and then assembled into functional molecules. Where tetravalent forms of antibodies are desired, the subunits will then self-assemble into tetravalent antibodies (WO02/096948A2).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278, in which the antibody coding sequence may be ligated individually into the vector in frame with the lacZ coding region so that a fusion protein is produced; pIN vectors; and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In addition to prokaryotes, eukaryotic microbes may also be used. Saccharomyces cerevisiae, or common baker's yeast, is the most commonly used among eukaryotic microorganisms although a number of other strains are commonly available, e.g., Pichia pastoris.

For expression in Saccharomyces, the plasmid YRp7, for example, is commonly used. This plasmid already contains the TRP1 gene which provides a selection marker for a mutant strain of yeast lacking the ability to grow in tryptophan, for example ATCC No. 44076 or PEP4-1. The presence of the trpl lesion as a characteristic of the yeast host cell genome then provides an effective environment for detecting transformation by growth in the absence of tryptophan.

In an insect system, Autographa californica nuclear polyhedrosis virus (AcNPV) is typically used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

Once an antibody molecule of the invention has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of antibodies of the invention is disclosed in US 2002 0123057 A1.

Animal Models of Disease and Use of Mouse CD20 and IgG2a Antibodies

Therapies using anti-human CD20 antibodies for human disease have been described previously (see, e.g., U.S. Pat. No. 5,736, 137 to Anderson et al.; U.S. Pat. No. 6,846,476 to White; U.S. Pat. No. 6,896,885 to Hanna; U.S. Pat. No. 6,455,043 to Grillo-Lopez; and U.S. Pat. No. 6,399,061; see also Moloney et al., 1997. Blood 90: 2188-2195, each of which is incorporated herein by reference in its entirety).

In certain embodiments, the anti-mouse CD20 antibodies, or antigen binding fragments, variants, or derivatives thereof, of the present invention are contemplated for use in a subject, in particular, an animal model of disease. In one embodiment, the compositions of the present invention are used in a method to deplete B-cells in a non-human subject. In another embodiment, the anti-mouse CD20 antibodies, or antigen binding fragments, variants, or derivatives thereof are used in a method of determining the effects of B-cell depletion in an animal model of disease, the method comprising administering to the animal model of disease an amount of the anti-mouse CD20 antibodies, or antigen binding fragments, variants, or derivatives thereof and observing the effects of the compositions on the B cell population of the animal model of disease.

By “observing the effects” is meant that various physical, health, physiological, and/or morphological parameters of the subject receiving the compositions of the present invention are examined and/or measured. In certain embodiments, the values or observations for the parameters are compared to various subjects which have received control compositions (i.e., something other than the compositions of the present invention) or have remained completely untreated. The parameters that will be of interest will vary depending on the animal model and the composition that is administered. It is well within the ordinary skill in the art to determine which parameters to measure in a particular study and how to perform the measurement. In addition, any of the numerous assays described elsewhere herein. In certain embodiments, the parameters to be examined and/or measured include, but are not limited to, a reduction in tumor size, an increase or decrease in expression of a gene or gene product (e.g., a protein), change in a morphological or physical characteristic (e.g., change in condition of a tissue or organ from a disease to a non-disease state, change in skin condition, change in gait or movement), and change in a physiological parameter (e.g., change in serum levels of molecules or substances). Methods of observation and measurement of these parameters are routine. In one embodiment, the B-cell population is measured by methods known to one of ordinary skill in the art. One such method is by cell staining and FACS analysis, which is described in more detail elsewhere herein.

In another embodiment, the anti-mouse CD20 antibodies, or antigen binding fragments, variants, or derivatives thereof, are used in a method for testing therapeutic agents for use in treating diseases or disorders treatable by B cell depletion, or to determine if a particular disease or disorder is treatable by B-cell depletion or some other mechanism of action of the compositions of the present invention, the method comprising administering to an animal model of disease an amount of a composition of the present invention and observing the effects of the composition on the state of the disease animal model of disease. By way of example, the composition may be administered alone, conjugated to another agent (e.g., a therapeutic agent), simultaneously with another agent (e.g., a therapeutic agent), or within a period before or after administration of another agent (e.g., a therapeutic agent) to the subject.

In one aspect of the invention, IgG2a antibodies (e.g., mouse IgG2a antibodies) specific for a target antigen (preferably a mouse antigen) are used in a method of determining the effects of treatment with antibodies to that the target antigen in an animal model (preferably a mouse) of disease (preferably human disease). In one embodiment, the method comprises administering to an animal model of disease an amount of an IgG2a antibody and observing the effects of the composition on the animal model of disease. By way of example, the composition may be administered alone, conjugated to another agent (e.g., a therapeutic agent), simultaneously with another agent (e.g., a therapeutic agent), or within a period before or after administration of another agent (e.g., a therapeutic agent) to the subject.

In another aspect, the present invention is directed to a method of simulating (e.g. modeling) treatment of human disease in a non-human animal model. According to one embodiment, the method comprises administering to the non-human animal model of disease an amount of a composition comprising an antibody or antigen binding fragment thereof that specifically binds to a target antigen in the animal model. In a specific embodiment, the antibody or antigen binding fragment thereof comprises an IgG2a isotype heavy chain constant region or a fragment thereof. In a preferred embodiment, the non-human animal model of disease is a mouse. In one embodiment, the method further comprises observing the effects of the administration of the composition on the state of the disease in the animal model. In one embodiment, the effects that are observed are the effector functions that are associated with (e.g., elicited or induced by) the IgG2a antibodies in the disease model.

In certain embodiments, the human disease (e.g., as represented by the non-human animal model of disease) is a neoplastic disorder (e.g., cancers and malignancies). The neoplastic disorder may comprise solid tumors such as melanomas, gliomas, sarcomas, and carcinomas as well as myeloid or hematologic malignancies such as lymphomas and leukemias. Exemplary cancers include, but are not limited to, prostate, gastric carcinomas (e.g., stomach or colon), skin, breast, ovarian, lung and pancreatic, Kaposi's sarcoma, CNS neoplasms (capillary hemangioblastomas, meningiomas and cerebral metastases), melanoma, gastrointestinal and renal sarcomas, rhabdomyosarcoma, glioblastoma (e.g., glioblastoma multiforme), leiomyosarcoma, retinoblastoma, papillary cystadenocarcinoma of the ovary, Wilm's tumor or small cell lung carcinoma.

Exemplary hematologic malignancies include Hodgkins and non-Hodgkins lymphoma, as well as leukemias, including ALL-L3 (Burkitt's type leukemia), chronic lymphocytic leukemia (CLL) and monocytic cell leukemias; a variety of B-cell lymphomas, including low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia. It should be clear to those of skill in the art that these lymphomas will often have different names due to changing systems of classification, and that patients having lymphomas classified under different names may also benefit from the combined therapeutic regimens of the present invention.

Besides neoplastic disorders, the human disease (e.g., as represented by the non-human animal model of disease) can be an autoimmune disorder or abnormal immune responses. In some embodiments the immune disorders include, but are not limited to, allergic bronchopulmonary aspergillosis; Allergic rhinitis Autoimmune hemolytic anemia; Acanthosis nigricans; Allergic contact dermatitis; Addison's disease; Atopic dermatitis; Alopecia areata; Alopecia universalis; Amyloidosis; Anaphylactoid purpura; Anaphylactoid reaction; Aplastic anemia; Angioedema, hereditary; Angioedema, idiopathic; Ankylosing spondylitis; Arteritis, cranial; Arteritis, giant cell; Arteritis, Takayasu's; Arteritis, temporal; Asthma; Ataxia-telangiectasia; Autoimmune oophoritis; Autoimmune orchitis; Autoimmune polyendocrine failure; Behcet's disease; Berger's disease; Buerger's disease; bronchitis; Bullous pemphigus; Candidiasis, chronic mucocutaneous; Caplan's syndrome; Post-myocardial infarction syndrome; Post-pericardiotomy syndrome; Carditis; Celiac sprue; Chagas's disease; Chediak-Higashi syndrome; Churg-Strauss disease; Cogan's syndrome; Cold agglutinin disease; CREST syndrome; Crohn's disease; Cryoglobulinemia; Cryptogenic fibrosing alveolitis; Dermatitis herpetifomis; Dermatomyositis; Diabetes mellitus; Diamond-Blackfan syndrome; DiGeorge syndrome; Discoid lupus erythematosus; Eosinophilic fasciitis; Episcleritis; Drythema elevatum diutinum; Erythema marginatum; Erythema multiforme; Erythema nodosum; Familial Mediterranean fever; Felty's syndrome; Fibrosis pulmonary; Glomerulonephritis, anaphylactoid; Glomerulonephritis, autoimmune; Glomerulonephritis, post-streptococcal; Glomerulonephritis, post-transplantation; Glomerulopathy, membranous; Goodpasture's syndrome; Granulocytopenia, immune-mediated; Granuloma annulare; Granulomatosis, allergic; Granulomatous myositis; Grave's disease; Hashimoto's thyroiditis; Hemolytic disease of the newborn; Hemochromatosis, idiopathic; Henoch-Schoenlein purpura; Hepatitis, chronic active and chronic progressive; Histiocytosis X; Hypereosinophilic syndrome; Idiopathic thrombocytopenic purpura; Job's syndrome; Juvenile dermatomyositis; Juvenile rheumatoid arthritis (Juvenile chronic arthritis); Kawasaki's disease; Keratitis; Keratoconjunctivitis sicca; Landry-Guillain-Barre-Strohl syndrome; Leprosy, lepromatous; Loeffler's syndrome; lupus; Lyell's syndrome; Lyme disease; Lymphomatoid granulomatosis; Mastocytosis, systemic; Mixed connective tissue disease; Mononeuritis multiplex; Muckle-Wells syndrome; Mucocutaneous lymph node syndrome; Mucocutaneous lymph node syndrome; Multicentric reticulohistiocytosis; Multiple sclerosis; Myasthenia gravis; Mycosis fungoides; Necrotizing vasculitis, systemic; Nephrotic syndrome; Overlap syndrome; Panniculitis; Paroxysmal cold hemoglobinuria; Paroxysmal nocturnal hemoglobinuria; Pemphigoid; Pemphigus; Pemphigus erythematosus; Pemphigus foliaceus; Pemphigus vulgaris; Pigeon breeder's disease; Pneumonitis, hypersensitivity; Polyarteritis nodosa; Polymyalgia rheumatic; Polymyositis; Polyneuritis, idiopathic; Portuguese familial polyneuropathies; Pre-eclampsia/eclampsia; Primary biliary cirrhosis; Progressive systemic sclerosis (Scleroderma); Psoriasis; Psoriatic arthritis; Pulmonary alveolar proteinosis; Pulmonary fibrosis, Raynaud's phenomenon/syndrome; Reidel's thyroiditis; Reiter's syndrome, Relapsing polychrondritis; Rheumatic fever; Rheumatoid arthritis; Sarcoidosis; Scleritis; Sclerosing cholangitis; Serum sickness; Sezary syndrome; Sjogren's syndrome; Stevens-Johnson syndrome; Still's disease; Subacute sclerosing panencephalitis; Sympathetic ophthalmia; Systemic lupus erythematosus; Transplant rejection; Ulcerative colitis; Undifferentiated connective tissue disease; Urticaria, chronic; Urticaria, cold; Uveitis; Vitiligo; Weber-Christian disease; Wegener's granulomatosis and Wiskoft-Aldrich syndrome.

In certain embodiments, the subject to which a composition of the present invention is administered is an animal model of disease. In a particular embodiment, the animal model of disease is a rodent model, and more particularly, a mouse model.

Animal models of disease are known in the art, but the following non-limited examples of animal models are provided. Timmerman et al. describe the use of a 38C13 mouse tumor model and β2M knockout mice as models of B-cell lymphoma (Timmerman et al., 2001. Idiotype-encoding recombinant adenoviruses provide protective immunity against murine B-cell lymphomas. Blood. 97:1370-1377). DeVisser et al. describe the use of K14-HPV16 and HPV16/RAG-1^{-/-} mice as models of epithelial carcinogenesis (DeVisser K E, Korets L V, Coussens LM. 2005. Cancer Cell. 7:411-423). Shah et al. describe the use of thymoma, colon carcinoma, and other tumor models (e.g., tumor growth in wild-type versus B-cell deficient mice) (Shah S, Divekar A A, Hilchey S P, Cho H M, Newman C L, Shin S U, Nechustan H, Challita-Eid P M, Segal B M, Yi K H, Rosenblatt J D. 2005. Int J Cancer. 117:574-586). Nabozny and David describe an experimental model for collagen induced arthritis (Nabozny G H, David C S. 1994. The immunogenetic basis of collagen induced arthritis in mice: an experimental model for the rational design of immunomodulatory treatments of rheumatoid arthritis. Adv Exp Med Biol. 347:55-63). All of the above references are herein incorporated by reference in their entireties.

Experimental autoimmune encephalomyelitis (EAE), which is an inflammatory demyelinating central nervous system disease that can be induced in rodents, e.g., by immunization with myelin proteins or passive transfer of activated CD4+ T cells for the proteins serve as the main animal model of human multiple sclerosis. EAE models are described in the following references, each of which is incorporated herein by reference in its entirety: Behi ME, et al. 2005. Immunol Lett. 96:11-26; Cross AH, et al. 2001. J Neuroimmunol. 112:1-14; Lyons J A, et al. 1999. Eur J Immunol. 29:3432-3439.

Animal models have also been described for human inflammatory arthritis. Kouskoff et al. describe a spontaneous mouse model of human rheumatoid arthritis generated by crossing a T-cell receptor transgenic line with the NOD strain of mice (KRNxNOD mice) (Kouskoff V, et al. 1996. Cell. 87:811-822). Lee et al., describe the use of the K/BxN model of destructive arthritis for the study of mast cell-dependent inflammation (Lee D M, et al. 2002. Science. 297:1689-1692). The above references are herein incorporated by reference in their entireties.

Animal models of human systemic lupus erythematosus (SLE) have also been described. Furukawa and Yoshimasu describe animal models (e.g., autoimmune MRL/lpr mouse) of spontaneous and drug-induced cutaneous lupus erythematosus (Furukawa F, Yoshimasu T. 2005. Autoimmun Rev. 4:345-350). Sanis and Datta describe the use of the NZB x SWR model of lupus nephritis (Sainis K, Datta S K. 1988. J Immunol. 140:2215-2224). Datta et al. describe the use of three mouse models of SLE (B/WF₁, SNF₁, and MRL-lpr mice)(Datta S K, et al. 1987. J Exp Med. 165:1252-1268. The above references are herein incorporated by reference in their entireties.

Animal models of human fibrosis diseases have also been described. Novobrantseva et al. describe a CCl₄-induced mouse model of liver fibrosis (Novobrantseva T I, et al. 2005. J Clin Invest. 115:3072-3082). Klahr and Morrissey describe an animal model of renal fibrosis (unilateral ureteral obstruction) (Klahr S, and Morrissey J. 2002. Am J Physiol Renal Physiol. 283(5):F861-875). Daniels et al. describe a bleomycin-induced lung fibrosis (e.g., idiopathic pulmonary fibrosis) (Daniels et al. 2004. J Clin Invest. 114:1308-1316).

Additional non-limiting examples of animal models of human disease are known and can be used with the antibodies and methods of the present invention. For example, a dextran sulfate-induced mouse colitis model simulates Crohn's disease and/or ulcerative colitis in humans. See, e.g., Murthy et al., Aliment. Pharmacol. Ther. 1999: 13:251-260 (incorporated herein by reference in its entirety). Murthy et al., used anti-TNFα antibodies in combination with pentoxifylilne to determine the effects of combination therapy. In one embodiment of the present invention, anti-mouse CD20 antibodies (e.g., of the IgG2a isotype) are used in the mouse colitis model to simulate the effects of treatment of a human with IgG1 anti-CD20 antibodies. According to one embodiment, the anti-mouse CD20 IgG2a antibodies are administered to the mouse colitis model and the effects on various parameters, including effector functions, are observed. The observations can be used, e.g., to predict the efficacy or safety of an analogous therapy in humans. In one embodiment, the anti-mouse CD20 IgG2a antibodies are administered in combination with other therapies (e.g., anti-TNFα antibodies and/or pentoxifylilne).

Lammerts van Bueren et al. showed, using animal models of EGFR over-expressing tumors, that clearance rates of antibodies against EGFR can play a role in the dose-effect relationship of therapy using anti-EGFR antibodies. Lammerts van Bueren et al, Cancer Res. 2006; 66:7630-7638 incorporated by reference herein in its entirety). Similarly, according to another embodiment of the present invention, IgG2a antibodies against, e.g., mouse CD20, are administered to an animal (e.g., mouse) model of mouse CD20-overexpressing tumors. In a particular embodiment, the effects of IgG2a isotype on clearance rates of the antibodies are observed. The observations can be used, e.g., to predict the efficacy or safety of an analogous therapy in humans.

A collagen-induced mouse model of arthritis simulates rheumatoid arthritis in humans. Plater-Zyberk et al., used anti-sera against IL-18 to determine the effects of IL-18 neutralization on the collagen-induced mouse model of arthritis. Plater-Zyberk et al., J Clin. Invest. 2001: 108:1825-1832 (incorporated by reference herein in its entirety). According to another embodiment of the present invention, IgG2a antibodies, preferably monoclonal mouse IgG2a anti-CD20 antibodies, are administered to a mouse model of rheumatoid arthritis. In other embodiments, the antibodies are administered in combination with other therapies.

The compositions and methods of the present invention can be used with each and any of these animal models of disease, and or with any other animal (particularly mouse) model of disease.

Pharmaceutical Test Compositions and Administration Methods

Methods of preparing and administering anti-mouse CD20 antibodies (or mouse IgG2a antibodies), or antigen-binding fragments, variants, or derivatives thereof of the invention to a subject in need thereof are well known to or are readily determined by those skilled in the art. The route of administration of the anti-mouse CD20 antibody (or mouse IgG2a antibody), or antigen-binding fragment, variant, or derivative thereof may be, for example, oral, parenteral, by inhalation or topical. The term parenteral as used herein includes, e.g., intravenous, intraarterial, intraperitoneal, intramuscular, subcutaneous, rectal or vaginal administration. While all these forms of administration are clearly contemplated as being within the scope of the invention, a form for administration would be a solution for injection, in particular for intravenous or intraarterial injection or drip. Usually, a suitable pharmaceutical test composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, anti-mouse CD20 antibodies (or mouse IgG2a antibodies), or antigen-binding fragments, variants, or derivatives thereof of the invention can be delivered directly to the site of the adverse cellular population thereby increasing the exposure of the diseased tissue to the therapeutic agent.

As previously discussed, anti-mouse CD20 antibodies, or antigen-binding fragments, variants, or derivatives thereof of the invention may be administered in an amount to effect depletion of a population of B cells. B-cell population depletion can be measured and observed by methods that are known in the art, and/or described in more detail in the Examples herein, without undue experimentation. In this regard, it will be appreciated that the disclosed antibodies will be formulated so as to facilitate administration and promote stability of the active agent.

Preferably, pharmaceutical test compositions in accordance with the present invention comprise a pharmaceutically acceptable, non-toxic, sterile carrier such as physiological saline, non-toxic buffers, preservatives and the like. For the purposes of the instant application, “an effective amount” of an anti-mouse CD20 antibody (or mouse IgG2a antibody), or antigen-binding fragment, variant, or derivative thereof, conjugated or unconjugated, shall be held to mean an amount sufficient to achieve effective binding to a target and to achieve a desired goal benefit, e.g., to ameliorate symptoms of a disease or disorder in an animal model of disease or to detect a substance or a cell or a particular physiological parameter.

The pharmaceutical test compositions used in this invention may comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical test compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., an anti-mouse CD20 antibody or mouse IgG2a antibody, or antigen-binding fragment, variant, or derivative thereof, by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof.

Parenteral formulations may be a single bolus dose, an infusion or a loading bolus dose followed with a maintenance dose. These compositions may be administered at specific fixed or variable intervals, e.g., once a day, or on an “as needed” basis.

The amount of an anti-mouse CD20 antibody (or mouse IgG2a antibody), or fragment, variant, or derivative thereof that may be combined with the carrier materials to produce a single dosage form will vary depending upon the host treated and the particular mode of administration. The composition may be administered as a single dose, multiple doses or over an established period of time in an infusion. Dosage regimens also may be adjusted to provide the optimum desired response (e.g., a therapeutic or prophylactic response in a non-human subject to mimic the response for a corresponding therapy if administered to a human subject).

In keeping with the scope of the present disclosure, anti-mouse CD20 antibodies (or mouse IgG2a antibodies), or antigen-binding fragments, variants, or derivatives thereof of the invention may be administered to a subject (e.g., a non-human animal model of disease) in accordance with the aforementioned methods of administration in an amount sufficient to produce a desired effect. The anti-mouse CD20 antibodies (or mouse IgG2a antibodies), or antigen-binding fragments, variants, or derivatives thereof of the invention can be administered to a subject in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of anti-mouse CD20 antibodies (or mouse IgG2a antibodies), or antigen-binding fragments, variants, or derivatives thereof of the invention may prove to be particularly effective or may be of particular interest for study in an animal model of disease.

Effective doses of the compositions of the present invention vary depending upon many different factors, including means of administration, target site, physiological state of the subject, other medications administered, and whether what is the goal of the study in which the composition is being administered (e.g., testing a combination therapy for its effects in an animal disease model as a predictor of its efficacy or toxicity in a human subject, or testing the effect of B-cell depletion in a model of a particular disease or disorder). For B-cell depletion, the dosage can range, e.g., from about 0.0001 to 100 mg/kg, and more usually 0.01 to 10 mg/kg (e.g., 0.02 mg/kg, 0.25 mg/kg, 0.5 mg/kg, 0.75 mg/kg, lmg/kg, 2 mg/kg, 5 mg/kg, 10 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. Exemplary dosage schedules include 1-10 mg/kg or 15 mg/kg on consecutive days, 30 mg/kg on alternate days, or 10 mg/kg or 60 mg/kg weekly. In some methods, two or more monoclonal antibodies with different binding specificities are administered simultaneously, in which case the dosage of each antibody administered falls within the ranges indicated. In a particular embodiment, the compositions of the present invention are administered in an amount of 10 mg/kg, every other week.

Anti-mouse CD20 antibodies (or mouse IgG2a antibodies), or antigen-binding fragments, variants, or derivatives thereof of the invention can be administered on multiple occasions. Intervals between single dosages can be weekly, monthly or yearly. Intervals can also be irregular as indicated by measuring blood levels of target polypeptide or target molecule in the subject. In some methods, dosage is adjusted to achieve a plasma polypeptide concentration of 1-1000 μg/ml and in some methods 1-30 μg/ml or 25-300 μg/ml. Alternatively, anti-mouse CD20 antibodies (or mouse IgG2a antibodies), or antigen-binding fragments, variants, or derivatives thereof of the invention can be administered as a sustained release formulation, in which case less frequent administration is required. Dosage and frequency vary depending on the half-life of the antibody in the subject. The half-life of a anti-mouse CD20 antibody (or mouse IgG2a antibody) can also be prolonged via fusion to a stable polypeptide or moiety, e.g., albumin or PEG. In one embodiment, the anti-mouse CD20 antibodies (or mouse IgG2a antibodies), or antigen-binding fragments, variants, or derivatives thereof of the invention can be administered in unconjugated form. In another embodiment, the anti-mouse CD20 antibodies (or mouse IgG2a antibodies), or antigen-binding fragments, variants, or derivatives thereof of the invention can be administered multiple times in conjugated form. In still another embodiment, anti-mouse CD20 antibodies (or mouse IgG2a antibodies), or antigen-binding fragments, variants, or derivatives thereof of the invention can be administered in unconjugated form, then in conjugated form, or vice versa.

The compositions of the present invention may be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In one embodiment of the invention, the anti-mouse CD20 antibodies, or antigen-binding fragments, variants, or derivatives thereof are administered in such a way that they cross the blood-brain barrier. This crossing can result from the physico-chemical properties inherent in the anti-mouse CD20 antibody (or mouse IgG2a antibody) molecule itself, from other components in a pharmaceutical formulation, or from the use of a mechanical device such as a needle, cannula or surgical instruments to breach the blood-brain barrier. Where the anti-mouse CD20 antibody (or mouse IgG2a antibody) is a molecule that does not inherently cross the blood-brain barrier, e.g., a fusion to a moiety that facilitates the crossing, suitable routes of administration are, e.g., intrathecal or intracranial, e.g., directly into a chronic lesion of MS or EAE. Where the anti-mouse CD20 antibody (or mouse IgG2a antibody) is a molecule that inherently crosses the blood-brain barrier, the route of administration may be by one or more of the various routes described below. In some methods, antibodies are administered as a sustained release composition or device, such as a Medipad™ device.

The compositions may also comprise a anti-mouse CD20 antibody (or mouse IgG2a antibody) dispersed in a biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides, copolymers of L-glutamic acid and gamma-ethyl-L-glutamate; poly(2-hydroxyethyl-methacrylate), ethylene vinyl acetate, or poly-D-(−)-3hydroxybutyric acid.

Anti-mouse CD20 antibodies (or mouse IgG2a antibodies), or antigen-binding fragments, variants, or derivatives thereof of the invention can optionally be administered in combination with other agents e.g., to be tested for toxicity or for efficacy, e.g., in treating or having an effect on the disorder or condition in an animal model of disease. The agents can be administered simultaneously or in any order, or with a time interval in between.

Examples of combinations of agents (e.g. therapeutic agents) that can be administered with the compositions of the present invention include, but are not limited to: anti-CD19 agents, anti-CD21 agents, anti-CD22 agents, anti-CD23 agents (e.g., in Chronic Lymphocytic Leukemia), anti-CD80 agents (e.g., in non-Hodgkin's lymphoma, rheumatoid arthritis); with chemotherapy in oncology (e.g., with CHOP in non-Hodgkin's lymphoma and FCR/fludarabine plus cyclophosphamide in Chronic Lymphocytic Leukemia); with toll receptor antagonists (immunostimulatory oligonucleotides) in lymphoma and other cancers; with standard of care in various diseases. Additional examples of agents to be used in combination with the compositions of the present invention particularly in autoimmune animal models include, but are not limited to: BR3-Fc or other mechanisms of BAFF antagonism; anti-adhesion molecule antibodies (e.g., anti-ICAM-1, anti-LFA-1 (anti-CD11a), anti-α4 integrin); lymphotoxin beta receptor antagonists (e.g., LTβR-Ig), anti-CD40 ligand (CD154); anti-inflammatory agents.

Mouse CD20-specific antibodies can be also used to assay protein levels in a biological sample using classical immunohistological methods known to those of skill in the art. Other antibody-based methods useful for detecting protein expression include immunoassays, such as the enzyme linked immunosorbent assay (ELISA), immunoprecipitation, or western blotting. Suitable assays are described in more detail elsewhere herein.

By “assaying the expression level of mouse CD20 polypeptide” is intended qualitatively or quantitatively measuring or estimating the level of mouse CD20 polypeptide in a first biological sample either directly (e.g., by determining or estimating absolute protein level) or relatively (e.g., by comparing to the cancer associated polypeptide level in a second biological sample). Preferably, mouse CD20 polypeptide expression level in the first biological sample is measured or estimated and compared to a standard mouse CD20 polypeptide level, the standard being taken from a second biological sample obtained from an individual not having the disorder or being determined by averaging levels from a population of individuals not having the disorder. As will be appreciated in the art, once the “standard” mouse CD20 polypeptide level is known, it can be used repeatedly as a standard for comparison.

By “biological sample” is intended any biological sample obtained from an individual, cell line, tissue culture, or other source of cells potentially expressing mouse CD20. Methods for obtaining tissue biopsies and body fluids from mammals are well known in the art.

Immunoassays

Anti-mouse CD20 antibodies (or mouse IgG2a antibodies), or antigen-binding fragments, variants, or derivatives thereof of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich”immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art . Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads).

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-mouse antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest is conjugated to a labeled compound (e.g., ³H or ¹²⁵I) in the presence of increasing amounts of an unlabeled second antibody.

Anti-mouse CD20 antibodies (or mouse IgG2a antibodies), or antigen-binding fragments, variants, or derivatives thereof of the invention, additionally, can be employed histologically, as in immunofluorescence, immunoelectron microscopy or non-immunological assays, for in situ detection of cancer antigen gene products or conserved variants or peptide fragments thereof. In situ detection may be accomplished by removing a histological specimen from a subject, and applying thereto, e.g., a labeled anti-mouse CD20 antibody, or antigen-binding fragment, variant, or derivative thereof, preferably applied by overlaying the labeled antibody (or fragment) onto a biological sample. Through the use of such a procedure, it is possible to determine not only the presence of, e.g., mouse CD20 protein, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for mouse CD20 gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of binding to mouse CD20 or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled anti-mouse CD20 antibody, or antigen-binding fragment, variant, or derivative thereof. The solid phase support may then be washed with the buffer a second time to remove unbound antibody. Optionally the antibody is subsequently labeled. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, agarose, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-mouse CD20 antibody (or mouse IgG2a antibody), or antigen-binding fragment, variant, or derivative thereof may be determined according to well known methods. In one method, the binding affinity of anti-mouse CD20 antibodies is measured using labeled antibodies and Scatchard analysis (e.g., Scatachard analysis using saturation binding experiments to determine recepter number and affinity by measuring specific binding at various concentrations of labeled antibodies). Those skilled in the art will be able to determine operative and optimal assay conditions for each determination by employing routine experimentation.

The practice of the present invention will employ, unless otherwise indicated, conventional techniques of cell biology, cell culture, molecular biology, transgenic biology, microbiology, recombinant DNA, and immunology, which are within the skill of the art.

### EXAMPLES

### Example 1

### Generation and Characterization of Anti-mouse CD20 Antibodies

### Summary

The clinical success of the anti-CD20 antibody, rituximab, in treating B cell neoplasias has fostered an interest in expanding the clinical applications for anti-CD20, combining anti-CD20 therapy with other potentially synergistic drugs, and in further characterizing the in vivo effects and mechanism of B cell depletion. The present invention encompasses a mouse anti-mouse CD20 antibody, designated 18B12. In addition to the original IgG1 isotype of 18B12, IgG2b and IgG2c switch variants were isolated and characterized. The VL and VH sequences of the 18B12 antibody were determined and used to construct a mouse IgG2a isotype (see Example 2, below). The 18B12 antibody recognizes mouse CD20 but not rat CD20 and is capable of efficiently depleting B cells when administered intravenously to wild type mice. In one embodiment, an optimal dosing regimen for maintaining B cell depletion was determined to be intravenous administration of a 10 mg/kg dose every other week. The anti-mouse CD20 antibody of the present invention can be used, for example, to determine the effects of B cell depletion in mouse disease models.

The 18B12 mouse monoclonal antibody to mouse CD20, generated by immunizing CD20 knockout animals (that have no apparent immune-related defects) with CD20-expressing mouse cells, efficiently depletes B cells in wild type mice and can be used in a variety of disease models, both alone and in combination with other therapeutic approaches.

### Experimental Procedures

Cloning and expression of mouse CD20. Total RNA was prepared from a BALB/c mouse spleen using an RNAEasy kit (Qiagen, Valencia, Calif.) and cDNA synthesized using SuperScript reverse transcriptase (Invitrogen, San Diego, Calif.). Mouse CD20 was PCR amplified using primers based on the published sequence (Liang and Tedder, 2001), CD20-5 (ATGAGTGGACCTTTCCCAGCAGA) (SEQ ID NO: 18) and CD20-31 (TTAAGGAGCGATCTCATTTTCCACTGGCAAGG) (SEQ ID NO: 19). In order to introduce restriction sites for cloning into the N5K-Ctldectin-B7.1-Ig vector (containing an Idectin peptide tag at the 3′- end of the gene of interest) a second PCR reaction was performed using the following primers, mD20-cIEf (ACAGATCTCACCATGAGTGGACCTTTCCCAGCAGAG) (SEQ ID NO: 20) and mD20-cIEr(GTGCTAGCAGGACGATCTCATTTTCCACTGG) (SEQ ID NO: 21), with substrate from the first PCR reaction and Pfu polymerase (Invitrogen). The PCR product was gel purified and overlapping adenines added at the 3′ end by incubation with Taq polymerase (1 units; Invitrogen) at 72° C. for 15 minutes. The fragments were cloned into pGEM-T (Promega, Madison, Wis.) and transformed into XLBlue-1 competent cells. A clone having the correct sequence of mouse CD20 was digested with SalI (pGEM-T polylinker) and NheI (in one primer) and the N5K-CtIdectin-B7.1-Ig vector was digested with Sall and NheI. After treatment of the vector with CIP phosphatase the fragment was ligated with the vector and transformed into Top-10 competent cells (Invitrogen).

To produce a stable Chinese Hamster Ovary (CHO) cell clone expressing mouse CD20 a N5K-CtIdectin-B7.1-Ig vector with the correct CD20 sequence was digested with Pac-1, and 10 μg DNA fragments were electroporated into CHO cells (DG44, 4×10⁶ cells) using a Gene Pulser II (Biorad, Richmond, Calif.). A stable cell line was selected in the presence of 0.4 mg/ml geneticin. CD20 expression was screened by immunoblot analysis and confirmed by flow cytometry on saponin-treated cells using an antibody to the Idectin epitope tag.

To produce stable 300.18 and 70Z/3 cell clones expressing mouse CD20, the CD20 gene was PCR amplified using primers RT205 (CACCATGAGTGGACCTTTCCCAGCAGAG) (SEQ ID NO: 22) and RT203 (AGGAGCGATCTCATTTTFCCACTGGC) (SEQ ID NO: 23) and the N5K-CD20-CtIdectin-B7.1-Ig vector template. The PCR product was purified and ligated into the pLenti6/V5-based expression vector (V5 peptide as a C-terminal epitope tag; ViraPower Lentiviral Expression System, Invitrogen). Production of virions containing the expression vector and infection of 300.18 and 70Z/3 cells were performed according to the manufacturer's instructions. Stable clones were selected in the presence of blasticidin (8 μg/ml) and screened by immunoblot analysis. High expresser clones (300.18 #8, 70Z/3 #2 and #18) were selected and used for immunization and subsequent screening.

Immunization and Hzybridoma Production. CD20 knockout mice back-crossed onto the C57BI/6 background were provided by Dr. J. Anolik at the University of Rochester, Rochester, N.Y. (O'Keefe et al., 1998). Mice were used for immunization at 6-8 weeks of age. Mice used for the fusion were immunized intraperitoneally as follows. Mouse N22: three times with 70Z/3 cells (5×10⁶ in 50 μl PBS), twice with a CD20 peptide (CSHFLKMRRLELIQTSKPYV) (SEQ ID NO: 24) conjugated to keyhole limpet hemocyanin (10 μg; KLH), and twice with 300.18-mCD20 cells (5×10⁶ in 50 μl PBS). Mouse N31: Four times with 300.18-mCD20 cells (once with 5×10⁶ cells in Complete Freund's Adjuvant and three times with 5×10⁶ in 50 μl PBS) and twice with CD20 peptide-KLH (10 μg). Three days after the final boost with 300.18-mCD20 cells and CD20 peptide-KLH spleen cells from the immunized mice were fused with NS-1 myeloma cells according to standard protocol (Kohler and Milstein, 1975) and plated in Iscove's Modified Dulbecco's Medium (Irvine Scientific, Irvine, Calif.) supplemented with 10% heat inactivated fetal bovine serum (FBS), L-glutamine (Gibco-BRL, Bethesda, Md.), non-essential amino acids (Sigma Chemical Co., St. Louis, Mo.), sodium pyruvate (Sigma Chemical Co.), and gentamicin (Gibco-BRL) under HAT selection. Seven to 12 days after fusion supernatants were screened by flow cytometry for binding to NS-1 myeloma cells (found positive for mouse CD20 mRNA by PCR analysis). Supernatants from positive wells were re-screened by flow cytometry for binding to spleen cells from wild type C57B1/6 mice and CD20 knockout mice. A hybridoma designated 18B12 was found to recognize NS-1 and spleen cells from wild type but not CD20 knockout mice. The 18B12 hybridoma was expanded, subcloned, and found to produce a mouse IgG1/κ antibody (final clone designated 18B12-A1C3-H3). Cells were expanded in stationary cultures and the antibody purified from supernatant by affinity chromatography on Protein A-Sepharose.

The 18B12-A1C3-H3 clone was deposited with the American Type Culture Collection (“ATCC”) on Dec. 22, 2005, and was given the ATCC Deposit Number PTA-7299. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

Flow Cytometry and ELISA Reagents. Reagents for cell staining and ELISA assays were as follows. Anti-B220 (RA3-6B2, APC, PerCP, and FITC conjugated), FITC-anti-CD11b (M1/70), anti-CD16/CD32 (2.4G2), PE-anti-CD19 (1D3), PE-anti-CD21 (7G6), biotin-anti-CD23 (B3B4), APC-anti-CD3 (145-2C11), PE-anti-CD43 (S7), PE-anti-CD5 (53-7.3), FITC-anti-IgD (11-26c.2a), biotin-anti-mouse IgG1^{a} (10.9), biotin-anti-mouse IgG1^{b} (B68-2), anti-mouse IgG2a^{(a+b)} (biotin conjugated and unconjugated) (R19-15), biotin-anti-mouse IgG2a^{b} (5.7), anti-IgM (biotin and PerCP-Cy5 conjugated) (R6-60.2), and APC-streptavidin were obtained from BD-PharMingen (San Diego, Calif.). PE-anti-mouse IgG (recognizing IgG1, IgG2a, IgG2b, and IgG3) (cat# 115-115-164) was from Jackson ImmunoResearch (West Grove, Pa.). Anti-mouse IgG1^{(a+b)} (biotin and PE conjugated) (H143.225.8), biotin-anti-IgG2a^{a} (H106.771), anti-mouse IgG2b (unconjugated, biotin-, and PE-conjugated) (LO-MG2b), biotin-anti-mouse IgG3 (LO-MG3), biotin-anti-mouse kappa (187.1) and biotin-anti-mouse lambda (JC5-1) were obtained from Southern Biotechnology (Birmingham, Ala.).

Cell Staining and Flow Cytometry Analyses. All staining procedures were done in round bottom 96-well plates (Corning 3799) in FACS buffer (Dulbecco's PBS supplemented with 2% FBS, 0.05% sodium azide, 10% normal goat serum (heat inactivated), and 2.4G2 (1 ug/ml). None of the monoclonal secondary antibodies used recognized rat IgG2b (the 2.4G2 antibody). Cells (1×10⁵ to 1×10⁶) were incubated with primary or secondary antibodies for 45 minutes each on ice, washed between incubations, and resuspended at 1×10⁶ cells/ml in FACS buffer for analyses. Fluorescence was measured on a FACSArray or FACSCalibur and analyzed with BD FACSArray System Software or Cell Quest Pro software.

For quantification of free 18B12 antibody in the sera of mice, bleeds were taken from dosed mice and the sera were used to stain mouse CD20-transfected 300.18 cells. A standard curve of the isotype being quantified was run and sera sample values were compared to the standard curve to determine the concentration of free antibody. Various dilutions of sera were tested and staining was quantified by flow cytometry.

Isotype ELISA Assays. For detecting isotypes of 18B12 96-well plates (Immulon2 HP, #3655 Thermo Labsystems) were coated with capture antibody (2 μg/ml in 0.1 M sodium bicarbonate pH 9.6) and detected with HRP-conjugated anti-mouse Kappa chain (clone 187.1). Substrate (TMB, KPL #50-76-00) was added, the reaction quenched with 4 N sulfuric acid, and plates read on a Vmax plate reader (450-750 nm; Molecular Devices, Palo Alto, Calif.).

Antibody cloning and nucleotide sequence analysis. Primers for PCR amplification of 18B12 VH and VL regions were designed based on the N-terminal amino acid sequences. The protein sequences were compared to known mouse antibody sequences and nucleotide sequences of antibody VH and VL regions with identical or most similar amino acid sequences were used to establish hypothetical nucleotide sequences for the 18B12 genes. Two degenerate primers were designed, JH-G1 (GGGGGTGTCGTGCTAGCTG(A/C)(G/A)GAGAC(G/A)GTGA) (SEQ ID NO: 25) and VK5-3 (CAAATTGT(G/T)ATGTC(C/A)CAGTCT) (SEQ ID NO: 26). Hybridoma cells were used to prepare cDNA and used as templates for high fidelity Pfx polymerase. Primers VK5-3 or VK5-1 (CAAATTGTTATGTCCCAGTCT) (SEQ ID NO: 27) were used with VK3 (TGCAGCATCCGTACGTTTGATTTCCAGCTT) (SEQ ID NO: 28) to amplify the Vκ region and primers VH5 (CAGGTCCAACTGCAGCAGCCTGGGGCTGA) (SEQ ID NO: 29) and JH-G1 were used to amplify the Vγ1 region. PCR reactions gave a single band of the correct size. Each PCR product was cloned into pCR4 TOPO vector (Invitrogen) and overlapping adenines added by incubation of the PCR products with Taq polymerase (1 unit). Fragments were cloned into the vector using the manufacturer's instructions. Sequences were obtained using M13F and M13R primers. To confirm the sequences PCR products from independent PCR reactions were sequenced.

### Results

Antibody 18B 12 Recognizes Mouse CD20

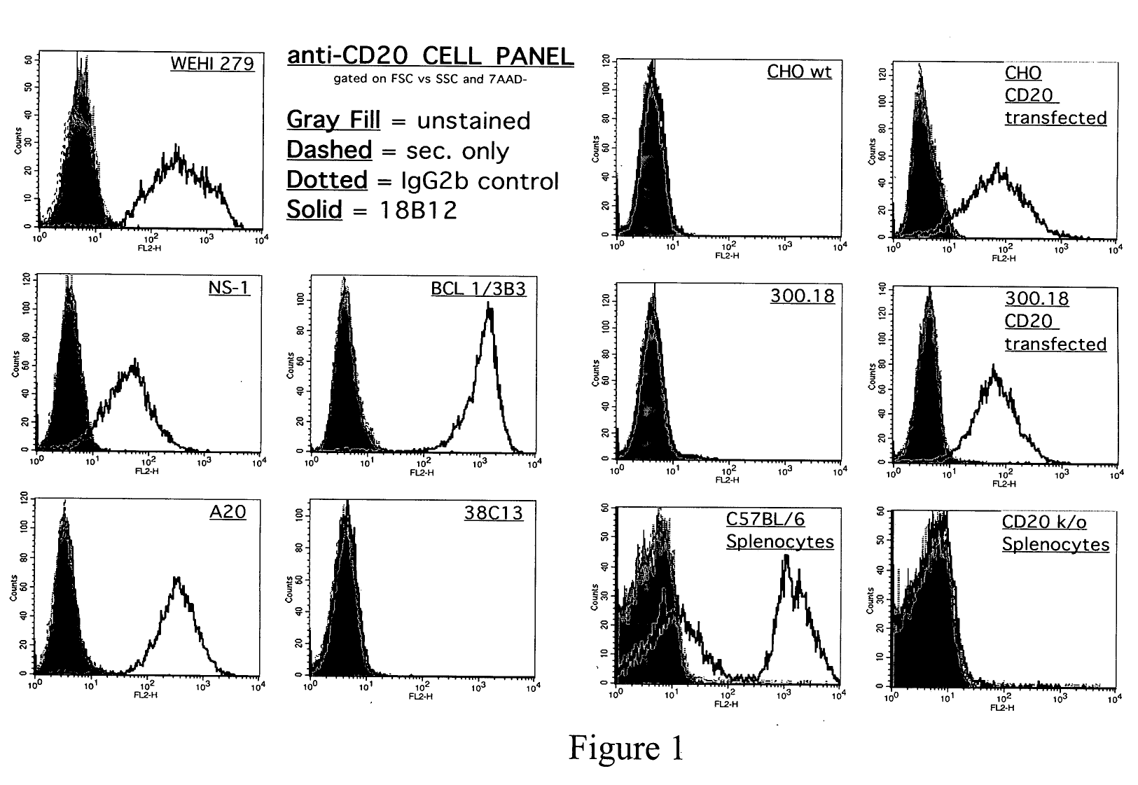

Fusion of splenocytes from two CD20 knock out mice that had been repeatedly immunized with mouse B cell lines (see Experimental Procedures) generated a set of hybridomas that recognized the NS-1 myeloma cell line. One IgG1-producing hybridoma, designated 18B12, had a staining profile that was consistent with specific recognition of mouse CD20. The 18B12 antibody bound to mouse B cell lines that expressed endogenous CD20 mRNA (WEHI 279, NS-1, A20), CHO and 300.18 cells that had been transfected with mouse CD20, but not to untransfected CHO and 300.18 cells (see FIG. 1, Table 4). Additionally the 18B12 antibody bound to CD19⁺ splenocytes from wild type C57B1/6 mice but not to splenocytes from CD20 knockout mice on the C57B1/6 background (see FIG. 1, Table 4). Since the antibody was produced in CD20 knockout mice it was possible that the antibody could recognize CD20 antigens on B cells from different rodent species, however 18B12 did not stain rat splenic B cells or two rat B cell lines (data not shown).

Switch Variants of 18B12 Exhibit the Same Specificity

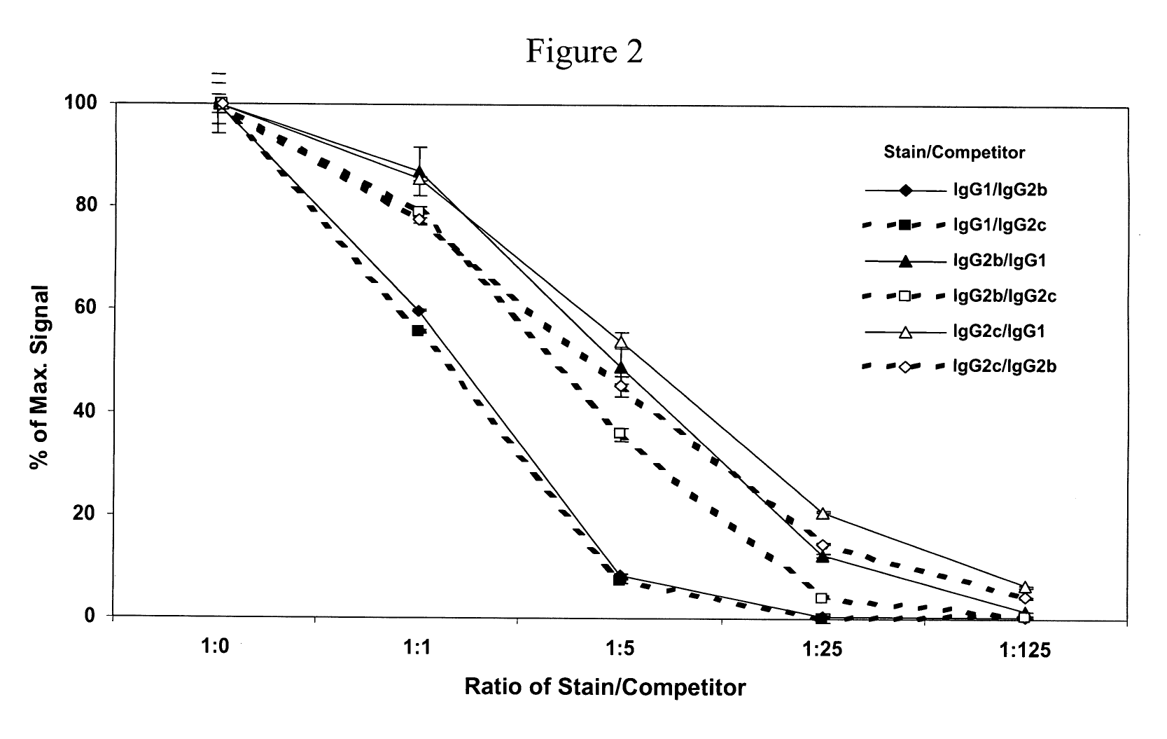

The 18B12 hybridoma was subcloned using limiting dilution methods and the supematant from subclones screened with monoclonal anti-mouse IgG2b to identify isotype switch variants. Weakly positive wells were further subcloned to isolate a population of cells that had completely switched isotype. Repetition of this procedure with the IgG2b variant of 18B12 followed by screening with an antibody recognizing both the “a” and “b” allotypes of IgG2a yielded a hybridoma cell line producing an IgG2c (IgG2a^{b} allele). The isotype switch variants exhibited an identical cellular staining pattern to the original 18B12 IgG1 antibody (shown in FIG. 1 and Table 4 for the IgG2b variant) and competed similarly to one another for binding to mouse CD20 transfected 300.18 cells (FIG. 2).

TABLE 4
Quantification of 18B12 Staining on Mouse B Cells^{a}

| | Geometric mean of anti-CD20 Staining | | | | | | | | | | |
|---|---|---|---|---|---|---|---|---|---|---|---|
| | | | | | | | | | | | 300.18- |
| Cell line | C57BI/6 wt | C57BI/6 k/o | WEHI 279 | NS-1 | BCL 1/3B3 | A20 | 38C13 | CHO wt | CHO-CD20 | 300.18 | CD20 |
| no stain | 3.6 | 3.9 | 5.2 | 3.8 | 4.1 | 3.5 | 4.0 | 4.0 | 3.6 | 4.2 | 4.0 |
| sec. only | 3.9 | 4.1 | 4.9 | 3.8 | 4.2 | 3.4 | 4.0 | 4.1 | 3.6 | 4.0 | 4.1 |
| IgG2b cont | 3.8 | 4.2 | 5.9 | 3.8 | 4.2 | 3.4 | 4.0 | 4.1 | 3.6 | 4.1 | 4.1 |
| 18B12 | | | 313.1 | 40.1 | 1024.1 | 333.5 | 4.1 | 4.1 | 61.8 | 4.0 | 61.8 |
| on CD19+ | 585.4 | 6.9 | | | | | | | | | |
| on CD19− | 7.3 | 4.0 | | | | | | | | | |

^{a}B cell lines or splenic B cells were stained with 18B12-IgG2b switch variant at 10 μg/ml and detected with a PE-anti-mouse IgG2b antibody as described in Experimental Procedures. Geometric means of the fluorescence intensities were calculated using CellQuestPro software.

Sequences of the 18B12 V_{H} and V_{L} Regions

To establish the native N-termini of the 18B12 heavy and light chain V regions the purified 18B12 IgG1 antibody was subjected to N-terminal sequence analysis. Both heavy and light chains were blocked by a pyroglutamic acid residue. This indicated that the N-terminal amino acid residues of both chains were glutamine. Protein sequences obtained after de-blocking the N-termini with pyroglutaminase (Table 5) were utilized to design 5′ primers for PCR amplification of the V_{H} and V_{L} genes from cDNA derived from 18B12 IgG1 hybridoma cells (ATCC Deposit No. PTA-7299).

TABLE 5

N-Terminal Sequence Analyses on the 18B12
Antibody^{α}

| | |
|---|---|
| V_{H(SEQ ID NO: 30)} | 1 10 21 |
| | (Q) VQLQQPGAE LVAPGTSVKL S |
| V_{L(SEQ ID NO: 31)} | 1 10 21 |
| | (Q) IVMSQSKAI XSAspXEKVT M |

^{α}N-terminal sequences were determined after de-blocking the antibody with pyroglutaminase.
Single letter amino acid abbreviations; lower case letters, lower confidence amino acid assignments; X, no amino acid could be assigned at this position. N-terminal glutamine residues were implied from blocked sequence.

The nucleotide sequences and translated protein sequences of the 18B12 V_{L} and V_{H} regions are described herein above and provided in the sequence listing at SEQ ID NO:1, SEQ ID NO:2, SEQ ID NO:3, and SEQ ID NO:4, respectively.

The 18B12 IgG1 Antibody Depletes B Cells in Wild Type C57B1/6 Mice

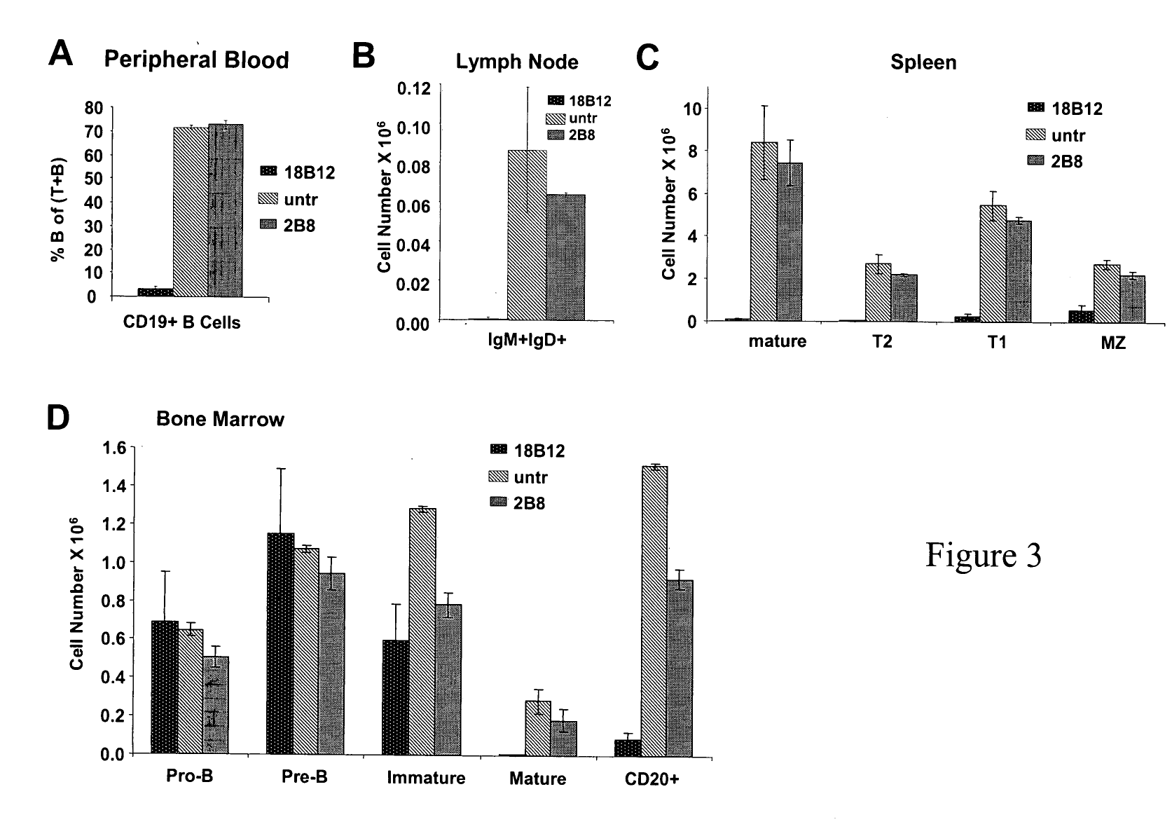

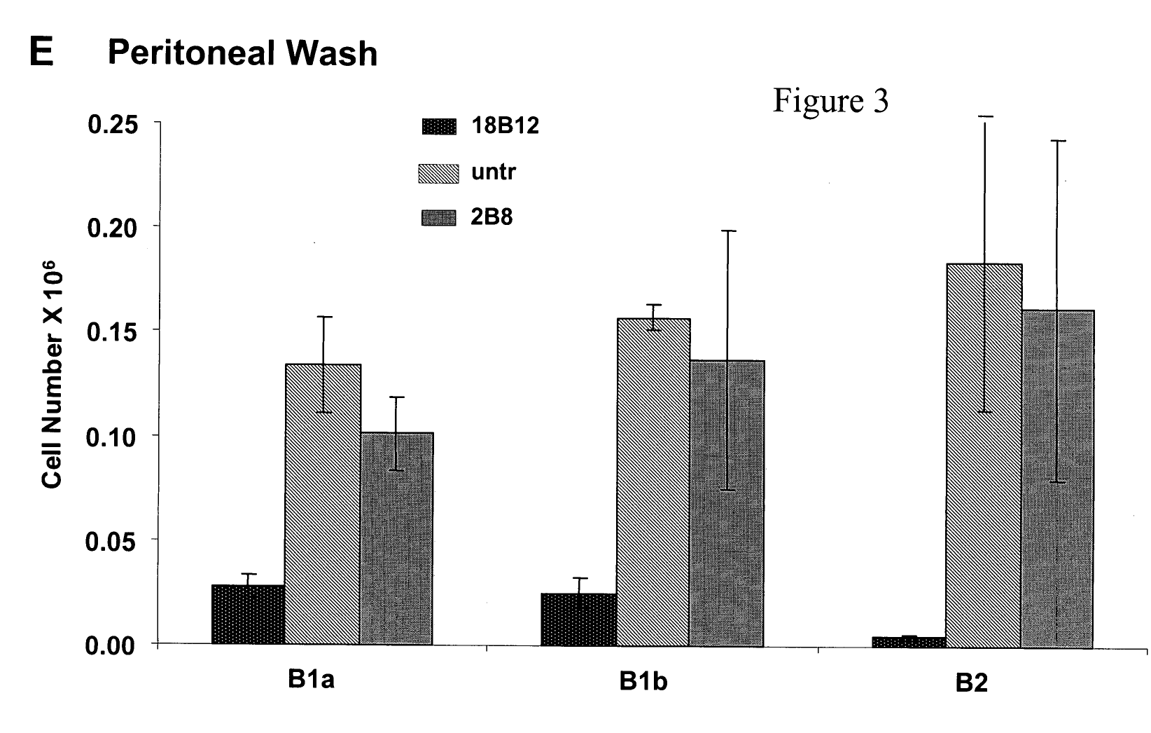

Initial experiments were performed to optimize the dosing regimen of the 18B12 IgG1 antibody for evaluation of its ability to deplete B cells in vivo. To parallel the in vivo administration of rituximab in human non-Hodgkin's lymphoma the 18B12 antibody was initially administered to mice intravenously (i.v.) at a dose of 10 mg/kg (rituximab is dosed to humans at 375 mg/m²). Analyses of the B cells in mouse peripheral blood indicated a dramatic decrease in B cell number three days after a single dose of 18B12. Dosing of 18B12 for optimal B cell depletion was found to require two i.v. injections at 10 mg/kg (day 0 and day 14). Major lymphoid tissues, including peripheral blood, lymph nodes, spleen, bone marrow, and peritoneal wash, were examined for the presence of B cell subsets identified by surface markers as described in Experimental Procedures (FIG. 3). B cells in peripheral blood were reduced by 97% (FIG. 3A). Similarly mature IgM^{lo}IgD^{hi} B cells in lymph nodes were depleted more than 98% (FIG. 3B). In spleen the majority of mature B cells, T2 B cells (IgM^{hi}IgD^{hi}), and T1 B cells (IgM^{hi}IgD^{lo}CD21⁻) were also depleted. However only ˜65-75% of the marginal zone (MZ) B cells (IgM^{hi}IgD^{lo}CD21⁺) were depleted by 18B12 treatment and a significant fraction remained in spleen (FIG. 3C). In bone marrow, pro B cells (B220^{lo}CD43⁺IgM⁻) and pre-B cells (B220^{lo}CD43⁻IgM⁻) were not depleted, while immature B cells (B220^{lo}CD43⁻IgM⁺) were only partially depleted (FIG. 3D). As assessed by 18B12 binding, pro B cells did not express CD20, although ˜10% of pre-B cells and ˜76% of immature B cells were CD20 positive. Similar to results in lymph node and spleen, the majority of mature B cells (>97%) in bone marrow were depleted following 18B12 treatment (FIG. 3D). In the peritoneal cavity B2 B cells (CD5⁻CD11b⁻B220^{hi}) were depleted by more than 97% after treatment with 18B12 but a fraction of B1a B cells (CD5⁺CD11 b⁺B220⁺; ˜28%) and B1b B cells (CD5⁻CD11b^{-B}220⁺; ˜18%) remained (FIG. 3E).

In Vivo B Cell Depletion by Different 18B12 Isotypes

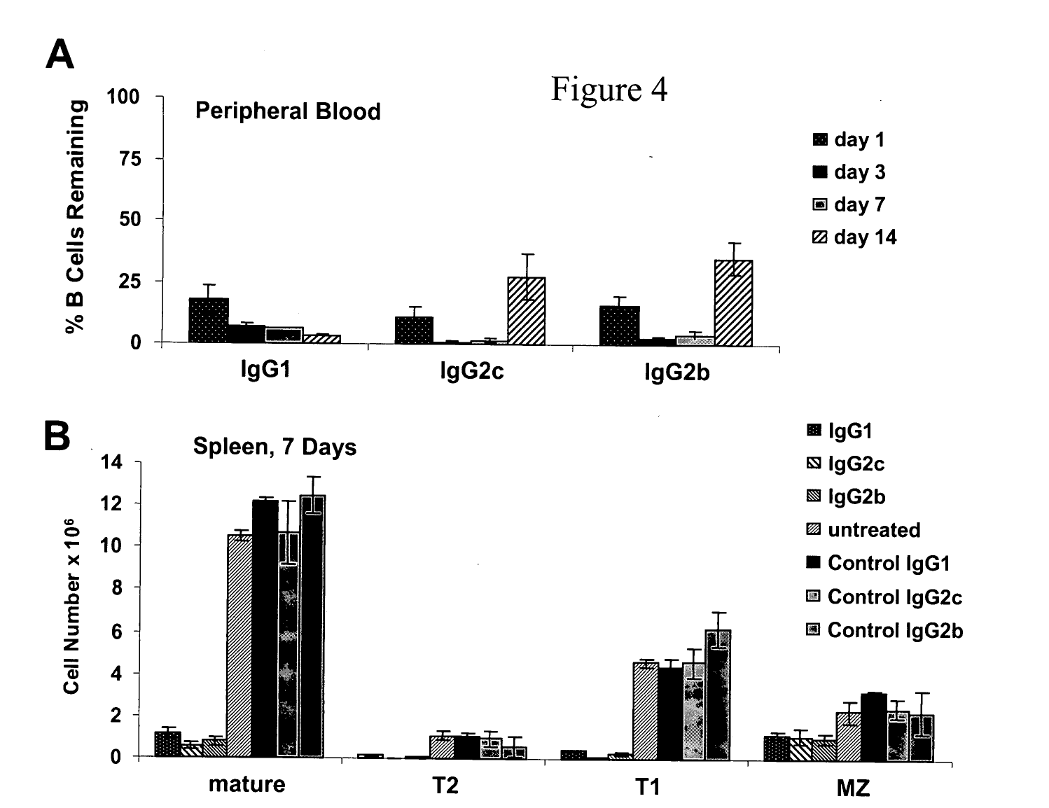

Different IgG isotypes have different affinities for the various Fcγ receptors (FcγR). Since FcγR are known to be involved in the in vivo Fc-dependent depletion of antibody-coated B cells, the IgG1, IgG2b, and IgG2c switch variants of 18B12 were compared for their ability to deplete B cells in wild type C57B1/6 mice. In peripheral blood the three isotypes of 18B12 depleted B cells to a similar extent at day one (11-18% of B cells remaining; FIG. 4A). By 3 days after treatment, the animals treated with the IgG2c and IgG2b antibodies appeared to have a more rapid and more complete peripheral blood B cell depletion as compared with animals treated with the IgG1 isotype. However, by 14 days after treatment while ˜97% of B cells were depleted in animals treated with the 18B12 IgG1, B cells in animals treated with the IgG2c or IgG2b isotypes were repopulating in the circulation (FIG. 4A). Therefore, the durability of B cell depletion appeared to be greater with 18B12 IgG1 treatment. In the spleen at day 7 all isotypes of 18B12 depleted the different B cell subsets to a similar extent (FIG. 4B). Similar to results shown in FIG. 4C, MZ B cells in the spleen were not efficiently depleted by any of the 18B12 isotypes (˜57% B cell depletion; FIG. 4B).

B Cell Repopulation after 18B12 IgG1 Treatment

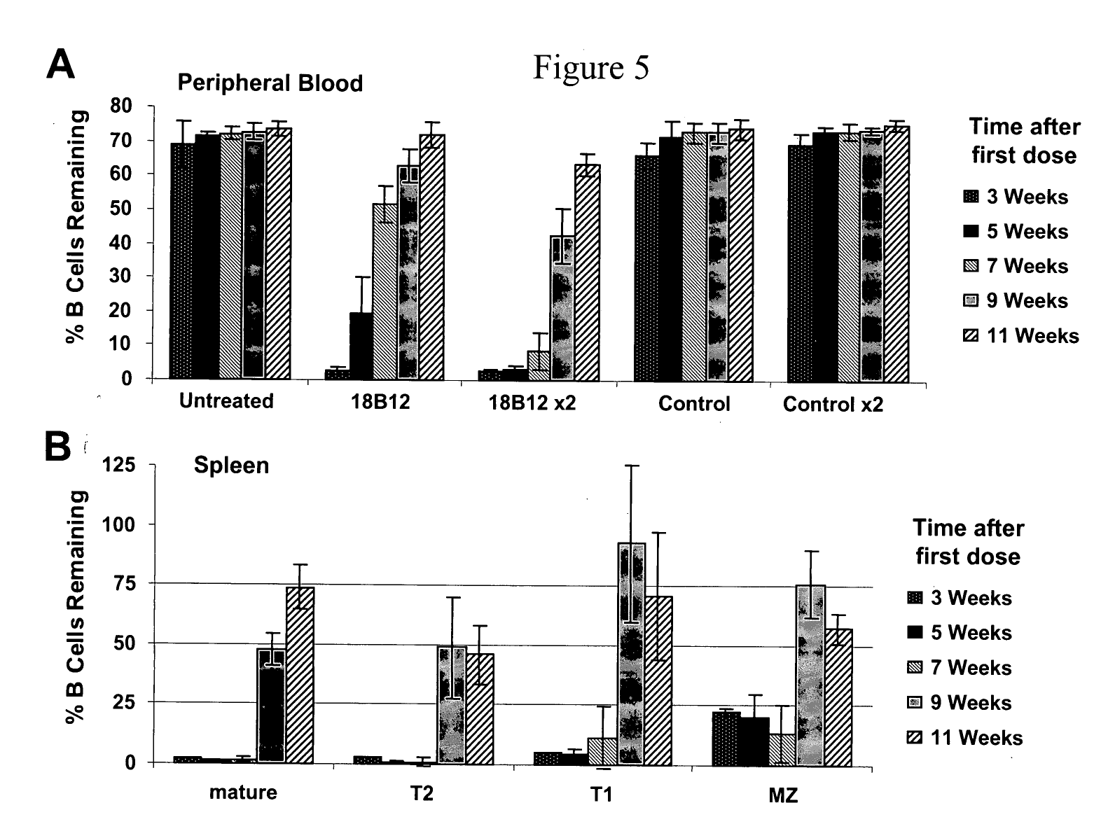

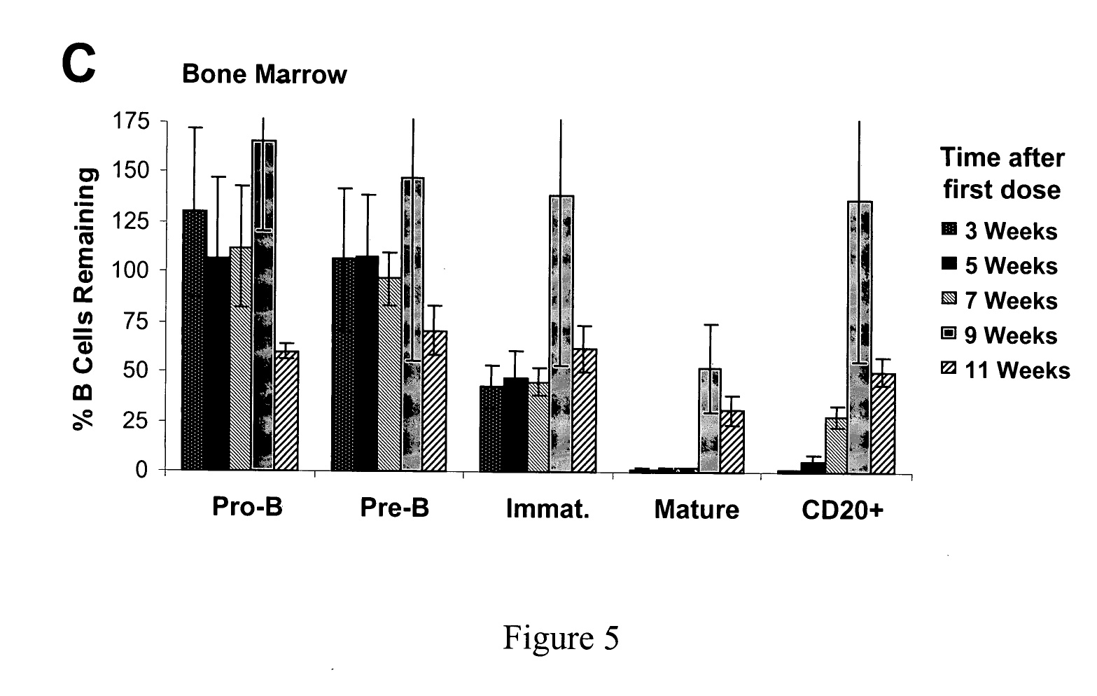

In humans treated with rituximab the duration of B cell depletion is long, with repopulation starting approximately 6 months after initial treatment. In contrast to human disease, many mouse disease models are acute, with rapid disease onset and progression. To optimize a B cell depletion regimen and develop dosing appropriate to disease models the duration of B cell depletion induced by 18B12 was examined. Wild type C57B1/6 mice were treated once or twice with 10 mg/kg 18B12 IgG1 and every two weeks, starting 3 weeks after the first treatment, B cell subsets were monitored in peripheral blood, spleen, and bone marrow (FIG. 5).

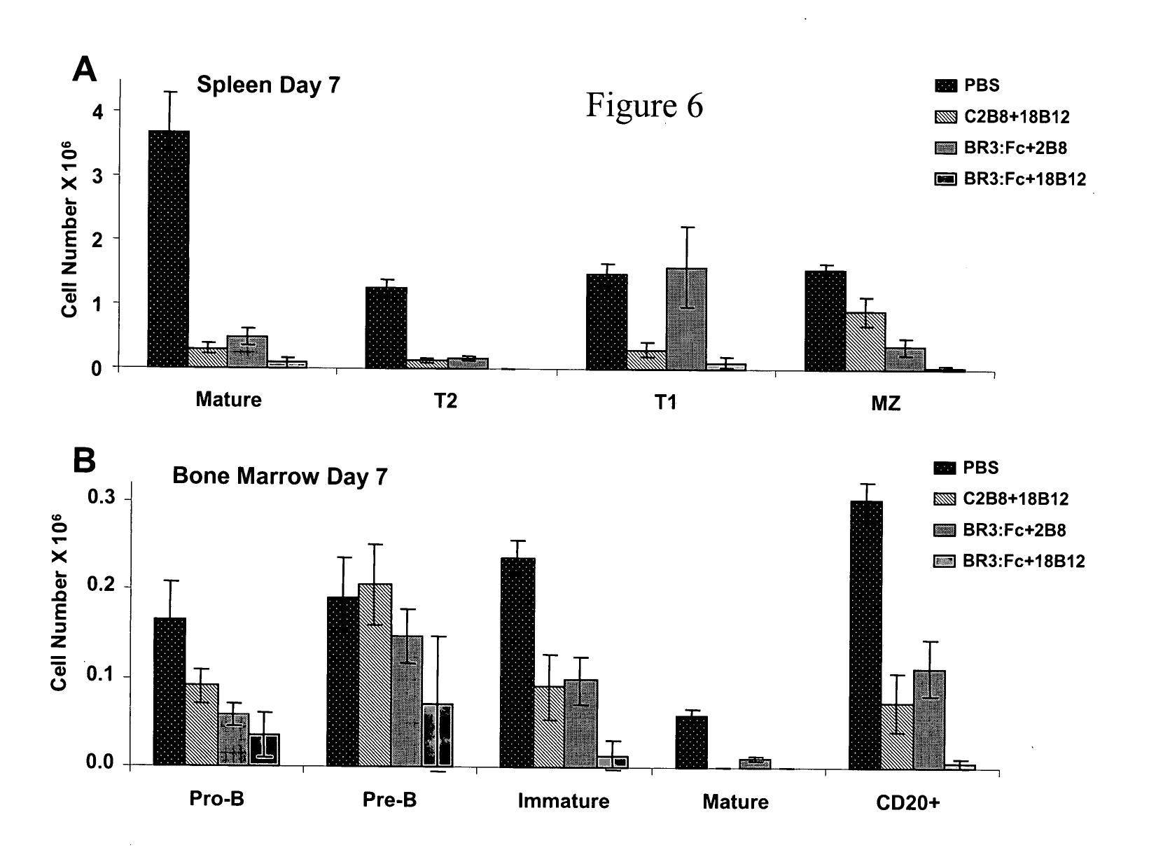

In peripheral blood B cells started to return by 5 weeks after a single 18B12 IgG1 dose and were almost recovered to levels in untreated animals by 9 weeks post-treatment (FIG. 5A). Animals given two doses of 18B12 on days 0 and 14 had B cell repopulation that was delayed from the single dosed animals by approximately two weeks (FIG. 6A). In spleens of animals given two doses of 18B12 on days 0 and 14, the T1 B cell subset showed the first indication of repopulation at 7 weeks and preceded the large increase in peripheral blood B cells at 9 weeks (FIG. 5A, B). These results are consistent with T1 being the earliest stage differentiated B cell in the spleen and with B cells repopulating via their normal developmental pathway from the bone marrow. Similarly in bone marrow of animals given two doses of 18B12, the last population to appear were the mature B cells at 9 weeks post-treatment (FIG. 5C).

The 18B12 IgG1 Antibody Depletes Marginal Zone B Cells Synergistically with BR3-Fc

BAFF is a tumor necrosis factor family member that is critical for B cell survival and binds to three known receptors, one of which is called BR3. A fusion protein of the BAFF receptor, BR3, with the Fc region of human IgG1 (BR3:Fc) was produced to be a soluble decoy receptor and has been shown to partially deplete mouse peripheral B cells, including marginal zone B cells (Biogen Idec internal report). To examine the effects of combining treatment with BR3:Fc and the 18B12 anti-mouse CD20 antibody, wild type mice were treated with either BR3:Fc, 18B12 antibody, or the combination (FIG. 6). Analyses of the B cells in the spleen one week after dosing showed that treatment with BR3:Fc or 18B12 depleted mature IgM^{lo}IgD^{hi} B cells and IgM^{hi}IgD^{hi} T2 B cells to a similar extent. The combination of the two agents depleted much more completely (FIG. 6A). BR3:Fc or 18B12 antibody partially depleted MZ B cells (78% and 42% depletion, respectively), however the combination of the two agents dramatically depleted MZ B cells (98% depletion; FIG. 6A). In bone marrow, the combination of BR3:Fc and 18B12 depleted a significant proportion of immature B220^{lo}CD43IgM⁺ B cells as compared with either agent alone (see also FIG. 3D) and resulted in more complete depletion of the CD20⁺cells (FIG. 6B). No significant differences in B cell depletion with BR3:Fc, 18B12, or the combination were observed in the peritoneal B cell subsets.

Pharmacokinetics of 18B12 Isotype Switch Variants

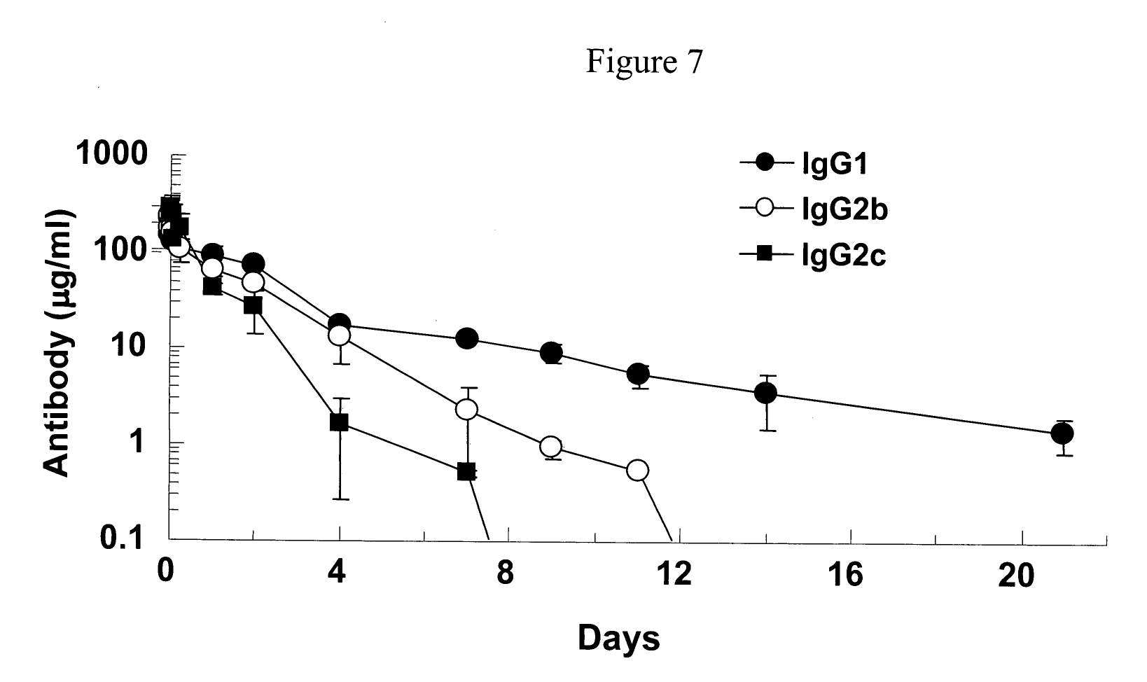

The serum pharmacokinetics of 18B12 IgG1, IgG2b, and IgG2c isotypes were examined in a single dose study. To parallel the B cell depletion study designs wild type C57B1/6 mice were given a single dose of 18B12 antibody at 10 mg/kg i.v. and blood was collected various times after injection for quantification of 18B12 antibody concentration in serum. Blood was also collected on days 1, 7, 14, and 21 for confirmation of B cell depletion. B cell depletion was found to be very similar to that shown in FIG. 4 (not shown). The results in FIG. 7 indicate the serum kinetics of each isotype differed. The IgGI isotype had the longest serum half-life and was still above 1 μg/ml at 21 days. The IgG2c isotype had the shortest serum half-life and reached the 1 μg/ml level approximately 5 days after dosing. The IgG2b isotype had an intermediate serum half-life and reached the 1 μg/ml level approximately 9 days after a single dose. These pharmacokinetic characteristics correlate with the duration of B cell depletion of each isotype (FIG. 4A) and suggest that a minimum serum concentration of anti-CD20 antibody is required to maintain B cell depletion.

### Summary and Conclusions

The present invention comprises the mouse monoclonal antibody that recognizes the mouse CD20 protein. That the 18B12 antibody is specific for mouse CD20 is indicated by binding of the antibody to two independent cell lines that were transfected with mouse CD20, with no binding to untransfected parental cell lines. Additionally, the 18B12 antibody bound to CD19⁺cells from wild type but not from CD20 knockout mice. IgG2b and IgG2c isotype switch variants of the original 18B12 IgG1 clone have been isolated and their binding and B cell depletion properties indicate they also recognize mouse CD20. The V_{L} and V_{F} sequences of the 18B12 antibody were determined and are unique sequences that were used to engineer a mouse IgG2a isotype (See Example 2, below).

The 18B12 antibody depleted mature B lymphocytes from peripheral blood, lymph nodes, spleen, bone marrow, and peritoneal cavity when administered i.v. to mice at 10 mg/kg, a dose that approximates a single dose of rituximab given to NHL patients. The IgG1 isotype efficiently depleted mature B cells from these tissues but did not deplete early B cells such as pro-B cells and pre-B cells in the bone marrow that have little or no CD20 expression. Additionally, the 18B12 antibody did not completely deplete immature B cells. Peritoneal B1a and B1b B cell subsets were not depleted as efficiently as the peritoneal B2 cells. They could be more resistant to depletion or regenerated at low levels independent of the bone marrow. In spleen the marginal zone B cell subset was incompletely depleted. These results are similar to those found with human CD20 transgenic mice treated with anti-human CD20 (Gong et al., 2005).

A single dose of 18B12 IgG1 depleted B cells for several weeks, with mature B cells returning in the circulation by 5 weeks post-treatment and a return to pre-treatment B cell levels by 9 weeks post-treatment. The IgG2b and IgG2c switch variants of 18B12 depleted B cells more rapidly than the IgG1, however B cells in animals treated with the IgG2b or IgG2c isotypes started to return in the peripheral blood as early as 2 weeks post-treatment. In one embodiment, an optimal B cell depletion protocol using the 18B12 IgG1 antibody would be to administer a 10 mg/kg dose i.v. every other week; in another embodiment, an optimal protocol for B cell depletion using the IgG2b or IgG2c isotypes would be to administer a 10 mg/kg dose i.v. on a weekly basis.

The small numbers of residual B cells in animals (and humans) following anti-CD20 treatment could be sufficient to retain a functional immune system but yet are not pathogenic.

### Example 2

### Generation and Characterization of an 18B12 IgG2a Antibody Isotype Variant

### Summary

Rituxan, an anti-human CD20 chimeric monoclonal antibody, has been shown to be effective in treating multiple human diseases, including non-Hodgkin's lymphoma and rheumatoid arthritis. As set forth in Example 1, above, a mouse IgG1 anti-mouse CD20 monoclonal antibody (18B 12), isolated IgG2b and IgG2c isotype switch variants, and demonstrated that all isotype variants depleted B cells in C57BL/6 mice to similar extents but had different half-lives and rates of B cell depletion. A study by Hamaguchi et al. showed that different antibody isotypes of anti-mouse CD20 had varying abilities to deplete B cells, generally IgG2a/c>IgG2b>IgG1 (Hamaguchi Y, Xiu Y, Komura K, Nimmerjahn F, and Tedder TF. 2006. J Exp Med. 203:743-753). However, the Hamaguchi et al. study used clonally distinct antibodies and did not control for the different epitopes and affinities of the anti-CD20 monoclonals tested. The present inventors engineered a mouse IgG2a anti-mouse CD20 monoclonal antibody using the V_{H} and V_{L} sequences of the original IgG1-secreting hybridoma, 18B12. To compare the B cell depletion characteristics of the 18B12 IgG2a isotype with that of the other characterized isotypes, normal BALB/c mice were dosed with either the IgG1, IgG2b switch variant, or engineered IgG2a variant of 18B12, and the resulting B cell depletion monitored. Although the three anti-CD20 monoclonals had identical V_{H} and V_{L} regions and binding characteristics, the heavy chain isotype had a profound effect on the ability to deplete B cells. Whereas mice treated with the IgG1 and IgG2b isotypes had resistant B cell subsets (limited depletion of splenic marginal zone B cells and peritoneal B1 B cells) the IgG2a-treated mice showed efficient and near complete depletion of all monitored B cell subsets by day 14. Even though affinity and epitope may play a significant role in the ability of different anti-CD20 antibodies to effect B cell depletion (Polyak & Deans, 2002), the profoundly stronger depletion capability of 18B 12 IgG2a compared to the IgG1 and IgG2b heavy chain isotypes demonstrates the importance of the Fc in eliminating B cells during anti-CD20 therapy.

### BACKGROUND

The mouse IgG1 anti-mouse CD20 antibody, 18B12, was derived from CD20 knockout mice bred onto the C57BL/6 genetic background (see Example 1, above). This mouse strain and several others including SJL and NOD produce the Igh1-b allele or allotype of IgG2a constant regions. Mice of the Igh1-b immunoglobulin allotype do not have a γ2a constant region gene, but instead produce the γ2c isotype from a distinct gene. Conversely, mice of the Igh1-a immunoglobulin allotype produce γ2a and do not have a γ2c gene. Because the spleen cell fusion to produce the 18B12 hybridoma derived from an Igh1-b allotypic background, the switch variant of the 18B12 IgG1-producing hybridoma has been identified as an IgG2c isotype. Since γ2a and γ2c gene sequences are 84% identical, antibodies containing these different constant regions may exhibit differences in effector function and may be immunogenic in the allotype non-identical mouse strain. In contrast to IgG2a and IgG2c allotypes of the Igh1 locus, IgG1 allotype differences at the Igh4 locus are minor, effector functions of these allotypes are expected to be similar, and sequence differences would not be expected to be immunogenic. Therefore, to utilize the 18B12 IgG2a/c isotype in Igh1-a allotype mouse strains, γ2a and κ constant regions were engineered onto the original 18B12 V_{H} and V_{L} sequences, respectively, and the IgG2a antibody expressed in Chinese hamster ovary (CHO) cells.

Mouse IgG2a antibodies are thought to be the functional equivalent of human IgG1 and have strong effector functions in vivo. The present inventors have engineered an IgG2a version of the 18B12 anti-mouse CD20 monoclonal and compared its B cell depletion characteristics with that of the IgG1 and IgG2b 18B12 isotypes.

### Experimental Procedures

Cloning and Expression of 18B12 IgG2a in CHO Cells. The V_{H} and V_{L} regions of the 18B12 IgG1 antibody cloned from the 18B12 hybridoma were subcloned into the N5mKmG2a vector (Biogen Idec) and used to transform the DG44dhfr⁻ CHO line using the Fugene 6 Transfection Kit (Roche). A stable high expressing subclone producing the IgG2a antibody was selected from bulk transfected cells by single cell sorting (MoFlo, Cytomation) and grown in Minimum Essential Medium Alpha Medium, containing L-glutamine and without ribonucleosides and deoxyribonucleosides (Invitrogen 12561-049, Carlsbad, Calif.). The resulting cell line was adapted to BCM16 medium for scale up production.

Isotype switch variants producing IgG2b and IgG2c were isolated from the original IgG1-producing 18B12 hybridoma as described previously (Spira et al., 1994).

Flow Cytometry Reagents. PerCP-labeled anti-B220 (RA3-6B2), unconjugated anti-CD16/CD32 (2.4G2), PE-anti-CD5 (53-7.3), FITC-anti-CD11b (M1/70), PE-anti-CD21 (7G6), FITC-anti-IgD (11-26c.2a), anti-IgM (biotin and PerCP-Cy5 conjugated) (R6-60.2), biotin-mouse anti-mouse IgGI (B68-2), biotin-rat anti-mouse IgG2a (R19-15), and APC-streptavidin were obtained from BD Biosciences (San Jose, Calif.). Biotin-rat anti-mouse IgG2b (LO-MG2b) was from Southern Biotech (Birmingham, Ala.), and 7-AAD was from Invitrogen.

Cell Staining and Flow Cytometry Analyses. All staining procedures were done in round bottom 96-well plates (Corning 3799) in FACS buffer ((Dulbecco's PBS supplemented with 2% FBS, 0.05% sodium azide, 10% normal goat serum (heat inactivated), and 2.4G2 (1 μg/ml)). None of the antibodies used recognized rat IgG2b (the 2.4G2 antibody). Cells (5×10⁵) were incubated with primary or secondary antibodies for 45 minutes each on ice, washed between incubations, and resuspended at 1×10⁶ cells/ml in FACS buffer for analyses. B cell subsets were defined according to the following phenotypic markers. Splenic B cell subsets: mature (B220⁺, IgM^{lo}, IgD^{hi}), marginal zone (B220⁺, IgM^{hi}, IgD^{lo}, CD21^{+, CD}23⁻), TI (B220⁺, IgM^{hi}, IgD^{lo}, CD21⁻, CD23⁻), and T2 (B220⁺, IgM^{hi}, IgD^{hi}, CD21⁺, CD23⁺). Peritoneal B cell subsets: B2 (B220^{hi}, IgM^{lo}, CD5⁻), Bla (B220^{lo}, IgM^{hi}, CD5⁺, CD11b⁺), and B1b (B220^{lo}, IgM^{hI}, CD5⁻, CD11b⁺). Fluorescence was measured on a FACSCalibur and analyzed with Cell Quest Pro software.

### Results and Conclusions

IgG1, IgG2b, and IgG2a Isotypes of 18B 12 have Identical CD20 Binding Characteristics

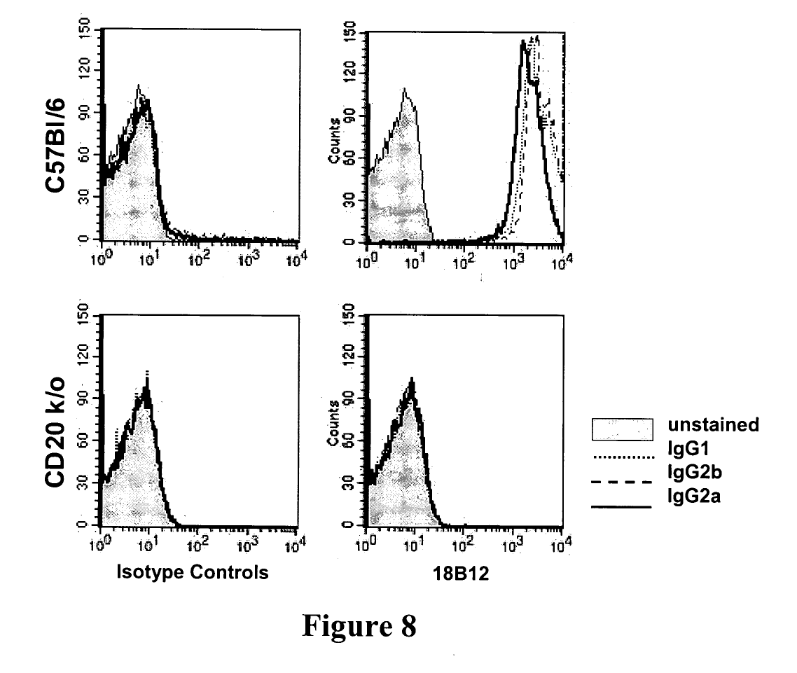

The original IgG1 18B12 anti-mouse CD20 antibody and the IgG2b and IgG2a isotype variants were used to stain spleen cells from either wild type C57BL/6 or CD20 knockout mice. The IgG2b switch variant and the engineered IgG2a have V_{H} and V_{L} regions identical to that of the original 18B12 IgG1. All isotypes exhibited similar cellular binding to CD19⁺ spleen cells from wild type mice with no apparent change in specificity (FIG. 8). There was no binding of any of the isotypes to CD19⁺ spleen cells from CD20 knockout mice (FIG. 8). The slight differences in staining intensity were due to variations in the different secondary antibodies used to detect the IgG isotypes, since the staining of a mouse CD20 transfected pre-B cell line (300.18) with the different isotypes using the same secondary detection reagent (anti-mouse kappa chain) produced identical histograms (not shown).

IgG2a 18B12 Exhibits Superior B Cell Depletion in BALB/c Mice

The efficacy of in vivo anti-CD20 therapy appears to be strongly dependent upon antibody Fc region interactions with Fcγ receptors. To compare the B cell depletion characteristics of the different 18B12 antibody isotypes containing identical V_{H} and V_{L} regions male BALB/c mice were administered a single dose of the IgG1, IgG2b, or IgG2a isotypes of 18B12 (10 mg/kg intravenously (i.v.)).

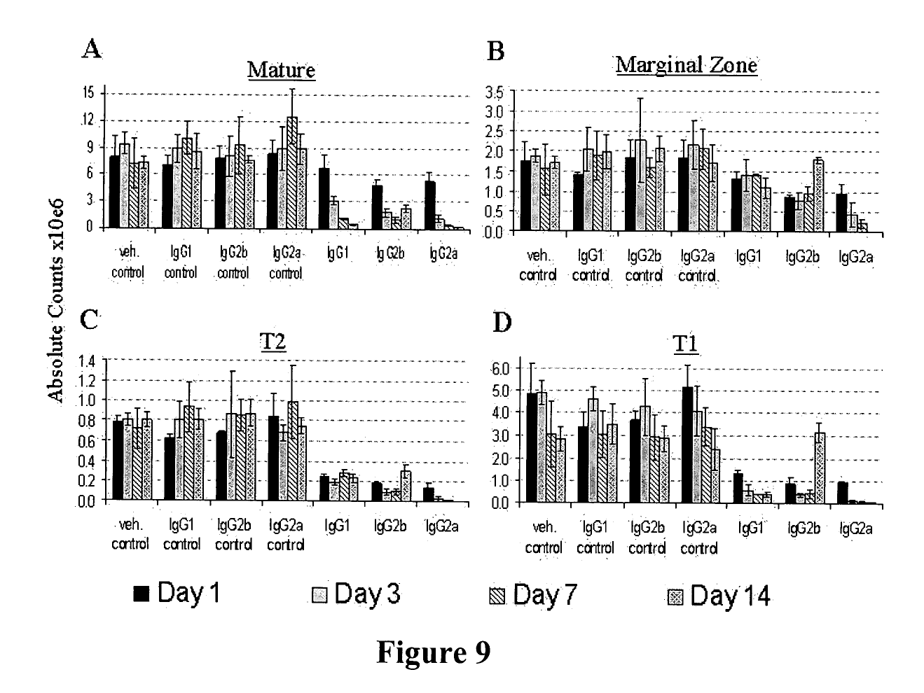

At day 1 post dosing, mice treated with any of the three 18B12 antibody isotypes showed similar depletion of the four splenic B cell subsets analyzed (mature, marginal zone, T2, and T1; FIG. 9). A summary of the percentages of each B cell subset remaining in the spleens of mice treated with the three different isotypes of anti-mouse CD20 is shown in Table 6. The IgG2b and IgG2a isotypes induced more efficient B cell depletion than the IgGI isotype (FIG. 9 and Table 6). Mature B cell depletion occurred gradually in IgG1- and IgG2b-treated mice and was near complete. At day 14, 95% of the mature B cells had been eliminated in animals treated with the IgGI isotype and at day 7 90% of mature B cells were eliminated in animals treated with the IgG2b isotype (Table 6). When marginal zone, T2, and T1 B cell subsets were monitored, both IgG1 and IgG2b isotypes showed rapid depletion capabilities with maximal B cell depletion attained at day 1 or day 3. However, marginal zone and T2 B cell depletion was only partial (in IgG1-treated mice, 23% and 66% depleted, respectively; in IgG2b-treated mice, 50% and 89% depleted, respectively; Table 6). During the first week of treatment the IgG2b isotype was at least as efficient as the IgG1 isotype in depleting the four B cell subsets studied and was better at depleting the marginal zone B cells. Due to the short half-life of the 18B12 IgG2b (32 hours; Table 7), antibody clearance resulted in B cell repopulation and a corresponding increase in B cell numbers by day 14 (FIG. 9; Table 6; Table 7).

In contrast to mice treated with either the IgG1 or IgG2b isotypes of 18B12, mice treated with the engineered IgG2a isotype demonstrated a progressive reduction in all four B cell subsets (mature, marginal zone, T2, and T1) and exhibited nearly complete splenic B cell depletion by day 14 (>99%; FIG. 9). B cell subsets previously resistant to depletion by the IgG1 or IgG2b isotype such as the marginal zone subset were efficiently eliminated from the spleens of mice treated with the IgG2a isotype. The extent of B cell depletion achieved in mice treated with the IgG2a isotype resembled that achieved previously with the combination of 18B12 IgG1 and BR3-Fc.

TABLE 6
B Cells Remaining After Treatment with 18B12 Isotype Variants (%)

| | Isotype Variant | | | | | | | | | | | |
|---|---|---|---|---|---|---|---|---|---|---|---|---|
| | IgG1 | | | | IgG2b | | | | IgG2a | | | |
| B Cell Subset | Day 1 | Day 3 | Day 7 | Day 14 | Day 1 | Day 3 | Day 7 | Day 14 | Day 1 | Day 3 | Day 7 | Day 14 |
| Mature | 87 | 35 | 12 | 5 | 63 | 21 | 10 | 28 | 68 | 13 | 4 | 1 |
| Marginal Zone | 77 | 67 | 80 | 58 | 50 | 37 | 54 | 96 | 56 | 20 | 13 | 1 |
| T2 | 34 | 24 | 32 | 29 | 25 | 11 | 12 | 37 | 20 | 3 | 2 | 0 |
| T1 | 31 | 13 | 12 | 14 | 20 | 8 | 13 | 110 | 23 | 2 | 2 | 1 |

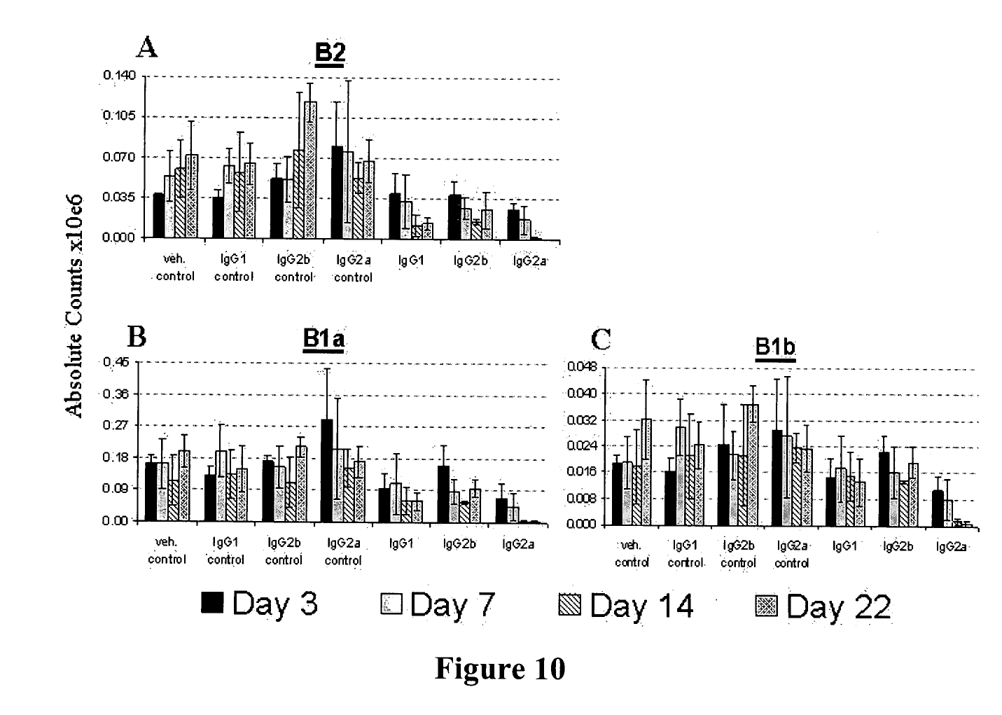

The populations of B cells in the peritoneal cavity, B2, B1a, and B1b, have previously demonstrated resistance to depletion with the IgG1, IgG2b, and IgG2c isotypes of 18B12, as well as with other anti-mouse CD20 antibodies (Hamaguchi et al., 2005). Therefore, in BALB/c mice treated with the IgG1, IgG2b, or IgG2a isotypes of 18B12 peritoneal B cell subsets were quantified. As found previously, the peritoneal B cell subsets of mice treated for 1 or 3 days with any of these three isotypes were only partially depleted (FIG. 10). In animals treated with the IgG1 or IgG2b isotypes maximal but partial peritoneal B cell depletion was found by day 7 (FIG. 10). In contrast, peritoneal B cell depletion mediated by the IgG2a isotype of 18B12 continued over time. By day 22 only residual numbers of all peritoneal B cell subsets were present in animals treated with the IgG2a isotype (FIG. 10).

In mice treated with the 18B12 IgG2a isotype, B cells were not observed to repopulate (monitored up to 22 days after treatment), and the approximate half-life of the 18B12 IgG2a in the blood was determined to be 7 days (data not shown). Compared with the half-life previously determined for the IgG1 (˜4.5 days), IgG2b (1.33 days) and IgG2c isotypes (<1 day; Table 7), the circulating half-life of the 18B12 IgG2a was considerably longer.

TABLE 7
Pharmacokinetics of 18B12 Isotype Variants

| Parameter¹ | Unit | IgG1 | IgG2b | IgG2c |
|---|---|---|---|---|
| AUClast | hr * μg/ml | 9008.91 | 5512.26 | 4081.42 |
| AUCinf | hr * μg/ml | 9226.37 | 5535.67 | 4097.02 |
| CL | ml/min/kg | 0.0181 | 0.0301 | 0.0407 |
| Kₑ | 1/hr | 0.0064 | 0.0214 | 0.0321 |
| T_{1/2} | hours | 107.66 | 32.44 | 21.63 |
| Cₘₐₓ | μg/ml | 215.5 | 233.2 | 286.3 |
| V_{dz} | L/kg | 0.1683 | 0.0845 | 0.0762 |

¹AUC, area under the curve; CL, clearance; Kₑ, partition coefficient; T_{1/2}, serum half-life; Cₘₐₓ, maximum concentration reached in serum; V_{dz}, volume of distribution.

In summary, treatment of mice with the IgG2a isotype of 18B12 resulted in more complete B cell depletion for all B cell subsets, including those previously resistant to depletion with the IgG1, IgG2b, and IgG2c isotypes. Because the V_{H} and V_{L} regions of the anti-CD20 monoclonal antibodies tested were identical these results demonstrate the superior efficacy of the IgG2a isotype over IgG1 and IgG2b isotypes in effecting the elimination of normal B cells subsets.

Without wishing to be bound by theory, the superior efficacy of the 18B12 IgG2a isotype in mediating depletion of B cell subsets could be due to several properties of this antibody. First, the 18B12 antibody itself could recognize an epitope on mouse CD20 that mediates more efficient B cell depletion (for example, positioning the antibody for more efficient Fcγ receptor engagement or Complement activation or increased cross-linking of CD20 on the cell surface) or the antibody V region could have a high affinity for mouse CD20. These possibilities are supported by comparing results generated on the 18B12 IgG1 isotype with those published by Hamaguchi et al., J Exp Med. 203:743-753 (2006).

The 18B12 IgG1 isotype was capable of depleting B cell subsets as efficiently as the anti-mouse CD20 IgG2a isotype antibody characterized by Hamaguchi et al. (2006), which was their most efficient B cell-depleting antibody. The two IgG1 isotype anti-mouse CD20 antibodies characterized by Hamaguchi et al. (2006) were less efficient in depleting B cells than the 18B12 IgG1 antibody. Second, the circulating half-life of the 18B12 IgG2a was longer than that of any other isotype tested, and cells in tissue compartments may be continuously exposed to greater antibody concentrations to effect better B cell depletion. Third, the IgG1 isotype of anti-mouse CD20 has been shown to mediate B cell depletion through the low affinity receptor FcγRIII, whereas the IgG2a and IgG2b isotypes have been demonstrated to mediate B cell depletion through a recently identified Fcγ receptor, FcγRPV. In vitro, FcγRIV exhibits little or no binding affinity for mouse IgG1; however it binds IgG2a and IgG2b with a moderate affinity, approximately 100-fold higher affinity than FcγRIII binds to IgG1. Since anti-CD20-mediated B cell depletion in mice appears to be mediated by the phagocytic network of the innate immune system, particularly monocytes and tissue macrophages, and both FcγRIII and FcγRIV are expressed by these cell types, higher affinity binding of the IgG2a isotype to FcγRfV could result in more efficient signaling and phagocytic effector function.

The present invention is not to be limited in scope by the specific embodiments described which are intended as single illustrations of individual aspects of the invention, and any compositions or methods which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference. U.S. Provisional Application Nos. 60/741,491, 60/783,060, and 60/849,433, are each incorporated herein in their entireties.

1. An isolated polynucleotide comprising a nucleic acid encoding an immunoglobulin heavy chain variable region (VH), wherein the CDR1, CDR2, and CDR3 regions of said VH are at least 95% identical, respectively, to reference heavy chain CDR1, CDR2, and CDR3 sequences of SEQ ID NO:8, SEQ ID NO:9, and SEQ ID NO:10, wherein an antibody or antigen-binding fragment thereof comprising said VH specifically binds to mouse CD20.

2. An isolated polynucleotide according to claim 1, wherein the CDR1, CDR2, and CDR3 regions of said VH, respectively, are identical to the sequences of SEQ ID NO:8, SEQ ID NO:9, and SEQ ID NO:10.

3. An isolated polynucleotide according to claim 1, further comprising a sequence encoding a mouse antibody heavy chain constant region or fragment thereof.

4. An isolated polynucleotide according to claim 3, wherein said heavy chain constant region is an IgG2a isotype constant region.

5. An isolated polynucleotide comprising a nucleic acid encoding a VH at least 90% identical to a reference VH sequence of SEQ ID NO:2 wherein an antibody or antigen-binding fragment thereof comprising said VH specifically binds to mouse CD20.

6. An isolated polynucleotide comprising a nucleic acid encoding an immunoglobulin heavy chain variable region (VL), wherein the CDR1, CDR2, and CDR3 regions of said VL are at least 95% identical, respectively, to reference heavy chain CDR1, CDR2, and CDR3 sequences of SEQ ID NO:5, SEQ ID NO:6, and SEQ ID NO:7, wherein an antibody or antigen-binding fragment thereof comprising said VL specifically binds to mouse CD20.

7. An isolated polynucleotide according to claim 6, wherein the CDR1, CDR2, and CDR3 regions of said VL, respectively, are identical to the sequences of SEQ ID NO:5, SEQ ID NO:6, and SEQ ID NO:7.

8. An isolated polynucleotide according to claim 6, further comprising a sequence encoding a mouse antibody light chain constant region.

9. An isolated polynucleotide comprising a nucleic acid encoding a VL at least 90% identical to the reference VL sequence of SEQ ID NO:1 or SEQ ID NO:32, wherein an antibody or antigen-binding fragment thereof comprising said VL specifically binds to mouse CD20.

10. A vector comprising the isolated polynucleotide of one of claim 1.

11. A host cell comprising the vector of claim 10.

12. A method of producing an antibody or an antigen binding fragment thereof that is capable of specifically binding to mouse CD20, said method comprising
a. culturing the host cell of claim 11 in a medium under conditions allowing the expression of said polynucleotide encoding said antigen binding molecule; and
b. recovering said antigen binding molecule from the resultant culture.

13. An anti-mouse CD20 antibody, or antigen-binding fragment thereof, produced by the method of claim 12.

14. An isolated polynucleotide comprising a nucleic acid encoding a heavy chain at least 90% identical to a reference VH sequence of SEQ ID NO:34, wherein an antibody or antigen binding fragment thereof comprising said VH specifically binds to mouse CD20.

15. An isolated polynucleotide comprising a nucleic acid encoding a heavy chain at least 90% identical to a reference VL sequence of SEQ ID NO:36, wherein an antibody or antigen binding fragment thereof comprising said VL specifically binds to mouse CD20.

16. An isolated polypeptide comprising an immunoglobulin heavy chain variable region (VH), wherein the CDR1, CDR2, and CDR3 regions of said VH are at least 95% identical, respectively, to reference heavy chain CDR1, CDR2, and CDR3 sequences of SEQ ID NO:14, SEQ ID NO:15, and SEQ ID NO:16, wherein an antibody or antigen-binding fragment thereof comprising said VH specifically binds to mouse CD20.

17. An isolated polypeptide according to claim 16, wherein the CDR1, CDR2, and CDR3 regions of said VH, respectively, are identical to the sequences of SEQ ID NO:14, SEQ ID NO:15, and SEQ ID NO:16.

18. An isolated polypeptide comprising a VH at least 90% identical to a reference VH sequence of SEQ ID NO:4 wherein an antibody or antigen-binding fragment thereof comprising said VH specifically binds to mouse CD20.

19. An isolated polypeptide comprising an immunoglobulin light chain variable region (VL), wherein the CDR1, CDR2, and CDR3 regions of said VL are at least 95% identical, respectively, to reference heavy chain CDR1, CDR2, and CDR3 sequences of SEQ ID NO:11, SEQ ID NO: 12, and SEQ ID NO:13, wherein an antibody or antigen-binding fragment thereof comprising said VL specifically binds to mouse CD20.

20. An isolated polypeptide according to claim 19, wherein the CDR1, CDR2, and CDR3 regions of said VL, respectively, are identical to the sequences of SEQ ID NO:11, SEQ ID NO:12, and SEQ ID NO:13.

21. An isolated polypeptide comprising a VL at least 90% identical to the reference VL sequence of SEQ ID NO:3, wherein an antibody or antigen-binding fragment thereof comprising said VL specifically binds to mouse CD20.

22. An antibody or antigen binding fragment thereof comprising one, two, three, four, five, or six CDRs of the 18B12 antibody, wherein said antibody or antigen binding fragment specifically binds to mouse CD20.

23. An antibody or antigen binding fragment thereof according to claim 22, wherein said antibody or antigen binding fragment comprises at least three CDRs of the 18B12 antibody.

24. A pharmaceutical test composition comprising the antibody or antigen binding fragment thereof according to claim 22.

25. A method of determining the effects of B-cell depletion in an animal model of disease, the method comprising:
a. administering to said animal model of disease an amount of a composition comprising the pharmaceutical test composition of claim 24; and
b. observing the effects of administration on said animal model of disease.

26. A method according to claim 25, wherein said animal model is a mouse.

27. A method according to claim 25, wherein observing effects of B cell depletion comprises a measurement selected from the group consisting of:
measuring the number of B-cells, measuring tumor size, measuring urine concentration of a protein or molecule, and measuring serum concentration of a protein or molecule.

28. A method according to claim 25, wherein said animal model of disease is a model for a human disease selected from the group consisting of B cell lymphoma, thymoma, colon carcinoma, epithelial carcinogenesis, collagen-induced arthritis, serum transfer arthritis, rheumatoid arthritis, mast cell-mediated inflammation, multiple sclerosis, systemic lupus erythrematosus, liver fibrosis, lung fibrosis, and kidney fibrosis.

29. A hybridoma cell line identified as American Type Culture Collection No. PTA-7299.

30. An antibody produced by the hybridoma cell line of claim 29.
